(19) United States
(12) Patent Application Publication
(10) Pub. No.: US 2010/0291584 A1
(43) Pub. Date: Nov. 18, 2010

(54) MICROFLUIDIC IMAGING CYTOMETRY

(75) Inventors: Hsian-Rong Tseng, Los Angeles, CA (US); Kenichiro Kamei, Los Angeles, CA (US); Jing Sun, Los Angeles, CA (US); Paul S. Mischel, Los Angeles, CA (US); Michael D. Masterman-Smith, Playa Del Rey, CA (US); David A. Nathanson, Playa Del Rey, CA (US); Tiffany Huang, Los Angeles, CA (US); Michael Van Dam, Glendale, CA (US); Christian Behrenbruch, Los Angeles, CA (US); Shawn M. Sarkaria, Los Angeles, CA (US)

Correspondence Address: VENABLE LLP, P.O. BOX 34385, WASHINGTON, DC, 20043-9998, US

(73) Assignee: The Regents of the University of California, Oakland, CA (US)

(21) Appl. No.: 12/863,526

(22) PCT Filed: Feb. 2, 2009
(86) PCT No.: PCT/US09/32880

§ 371 Date: Jul. 19, 2010

Related U.S. Application Data
(60) Provisional application No. 61/006,842, filed on Feb. 1, 2008.

(51) Int. Cl. C12Q 1/68; C12M 1/34; G01N 33/50

(52) U.S. Cl. 435/6; 435/288.7; 435/7.21

(57) ABSTRACT

A microfluidic system has a pipette system comprising a plurality of pipettes, a microfluidic chip arranged proximate the pipette system, an imaging optical detection system arranged proximate the microfluidic chip, and an image processing system in communication with the imaging optical detection system. The microfluidic chip has a plurality of cell culture chambers defined by a body of the microfluidic chip, each cell culture chamber being in fluid connection with an input channel and an output channel defined by the microfluidic chip. The pipette system is constructed and arranged to at least one of inject fluid through the plurality of pipettes into the plurality of input channels or extract fluid through the plurality of pipettes from the plurality of output channels while the microfluidic system is in operation.

This application claims priority to U.S. provisional application No. 61/006,842 filed on Feb. 1, 2008, the entire contents of which are incorporated herein by reference.

### BACKGROUND

1. Field of Invention

Embodiments of the present invention relate to microfluidic systems, and more particularly to microfluidic systems and methods for large-scale cell culture and assay.

2. Discussion of Related Art

All references cited in this specification are incorporated herein by reference.

### Problems of Brain Tumor Classification

Astrocytic brain tumors span a wide range of neoplasms with distinct clinical, histopathological, and genetic features. Molecular genetic data that has been gathered since the prior WHO classification in 1993 suggest that individual histologically defined types of astrocytomas are even more diverse at a biological level.¹ For instance, the majority of glioblastomas arise without clinical or histological evidence of a less malignant precursor lesion and these lesions have been designated primary glioblastoma. They appear in older patients (mean age, 55 yr) after a short clinical history of usually less than 3 months. These primary glioblastomas are characterized by EGFR amplification (˜40% of cases) and/or overexpression (60%), PTEN mutations (30%), p16INK4a deletion (30%-40%), MDM2 amplification (<10%) and/or overexpression (50%), and in 50%-80% of cases, loss of heterozygosity (LOH) on the entire chromosome 10. In contrast, secondary flioblastomas develop more slowly malignant progression from diffuse (WHO grade II) or anaplastic astrocytoma (WHO grade III) and appear in younger patients (mean age, 40 yr). Secondary glioblastomas contain TP53 mutations in approximately 60% of cases; more than 90% of these mutations are already present in the preceding diffuse (WHO grade II) or anaplastic astrocytoma (grade III). The pathway to secondary glioblastomas is further characterized by allelic loss of chromosomes 19q and 10q¹. Histopathologically, an unambiguous distinction of these subtypes has remained elusive, but they clearly evolve through different genetic pathways¹⁻³. It also remains to be shown whether these subtypes differ significantly with respect to prognosis, but it is likely that they will respond differently to specific novel therapies as they are developed⁴. As a result, ongoing clinical trials need to incorporate molecular su typing and future classification schemes will no doubt be based on such differences as well⁵.

### PI3K/Akt/mTOR Signaling Pathway in Brain Cancer

### EGFR and EGFRvIII

Among patients with glioblastoma, the most common primary malignant brain tumor of adults, small subgroup seems to benefit from the EGFR kinase inhibitors erolotinib and gefitinib⁶. However, the infrequency of mutations in the EGFR kinase domain in the glioblastomas^{7,8} suggests that such EGFR mutations cannot account for responsiveness to EGFR kinase inhibitors⁹. The EGFR gene is commonly amplified in glioblastoma¹⁰, but this abnormality also does not correlate with responsiveness to EGFP kinase inhibitors⁹, Glioblastoma often express EGFRvIII, constitutively active genomic deletion variant of EGFR¹¹⁻¹⁵. This variant of EGFR strongly and persistently activates the phosphatidylinositol 3′ kinase (PI3K) signaling pathway, which provides critical information for cell survival, proliferation, and motility¹⁶⁻²⁰. Persistent PI3K signaling activated by EGFRvIII is believed to cause pathway addiction²¹; addicted tumor cells die if the pathway is disrupted by tyrosine kinase inhibitors. By promotering chronic dependence on PI3K signaling, EGFRvIII may sensitize glioblastoma cells to EGFR kinase inhibitors.

### PTEN

The PTEN (phosphatase and tensin homologue deleted in chromosome 10) tumor-suppressor protein, inhibitor of the PI3K signaling pathway, is commonly lost in glioblastoma^{10,17,22}. This loss may promote cellular resistance to EGFR kinase-inhibitor therapy by dissociating EGFR inhibition from downstream PI3K pathway inhibition²³. We hypothesized that EGFRvIII would sensitize tumors to EGFR kinase inhibitors, whereas PTEN loss would impair the response to such inhibitors²³.

To test this hypothesis, we analyzed EGFRvIII and PTEN at the gene and protein levels in glioblastomas from patients before treatment with EGFR kinase inhibitors. We also searched for mutations in EGFR and in its heterodimerization partner Her2/neu, which has been reported to be mutated in glioblastoma and could also affect the response to EGFR kinase inhibitors²⁴. We found a strong association between the coexpression of EGFRvIII and PTEN in glioblastoma cells and responsiveness to EGFR kinase inhibitors.

mTOR

Mammalian target of rapamycin (mTOR, also known as FRAP, RAFT1 and RAP1) has been identified as a key kinase acting downstream of the activation of PI3K²⁵. Rapamycin and rapamycin derivatives that specifically block mTOR have been developed during the past 5 years as potential anticancer agents. mTOR regulates essential signal transduction pathways and is involved in coupling growth stimuli to cell-cycle progression. In response to growth-inducing signals, quiescent cells increase the translation of a subset of mRNAs, the protein products of which are required for progression through the G1 phase of the cell cycle. PI3K and AKT are the key elements of the upstream pathway that links the ligation of growth factor receptors to the phosphorylation and activation state of mTOR^{26,27}. With regard to the role of the PI3K/AKT/mTOR pathway in the genesis and proliferation of cancer cells, elements of the PI3K/AKT/mTOR pathway have been demonstrated to be activated by the erythroblastic leukaemia viral oncogene homologue (ERB) family of surface receptors, the insulin like growth factor receptors (GFRs), and oncogenic Ras²⁸⁻³¹.

### The Radiotracer for Positron Emission Tomography (PET)

The clinical applications of ¹⁸F-2-fluoro-2-deoxy-d-glucose (FDG) positron emission tomography (PET) are continually expanding, especially in the field of oncology. In colorectal cancer (CRC), the diverse uses of PET include initial diagnosis, staging, restaging, and assessment of the therapeutic response³²⁻³⁴. PET has also been reported to offer advantages over conventional, anatomically based morphologic modalities for detecting recurrent CRC and metastatic disease, because of its capacity to provide a functional image and evidence of tumor behavior^{35,36}. This is based on the knowledge that enhanced glucose uptake is one of the major metabolic changes characteristic of malignant tumors. Clinically, FDG is the most commonly used PET tracer. It is metabolized similarly to glucose, being transported into the cell, but once enzymatically phosphorylated, FDG-6-phosphate is metabolically trapped in tumor cells. Thus, tumors demonstrate increased commutation of FDG and can be distinguished on PET scan images by areas of increased tracer activity³⁷. Clinically, variable FDG uptake, semiquantified as the standardized uptake value (SUV), has been seen on PET scans of tumors from the same origin, including CRC. Much research has been done on the differences in FDG uptake among tumors and the mechanism of this uptake. Emerging evidence indicates that the factors affecting FDG uptake are complicated because the specific biological characteristics of tumors determine the degree of glucose metabolism³⁸⁻⁴⁰. Most factors affecting FDG uptake, such as hypoxia and cell density, are thought to be associated with changes in glycolysis-related protein expression^{41,42}. The expression of glucose transporter proteins, especially GLUT-1, which is directly involved in FDG uptake, is thought to determine the levels of FDG uptake in cancer cells⁴³⁻⁴⁵.

### PI3K/Akt/mTOR Signaling Pathway and PET Probe Uptake

As described above, FDG is the most commonly used as a PET tracer, and transported into cells through glucose transporter (Glut). This transported FDG was phosphorylated by Hexokinase and trapped in cells. Thus FDG uptake was regulated by both Glut and hexokinase. However, interestingly, localization of Glut to cell membrane is enhanced by Akt activity. Additionally, expression of hexokinase was also regulated by Akt. This means FDG uptake is strongly related with PI3K/Akt/mTOR pathway.

In a limited number of pilot trials using RAD001 and AP23573, 18F-2-fluoro-2-deoxy-d-glucose positron emission tomography (PET) was used to monitor the glucose uptake of tumors following administration of mTOR inhibitors. In those preliminary studies, some tumors exhibited a decrease in glucose uptake that was not consistently associated with objective responses determined by radiological methods⁴⁶.

### Drug Screening and PET Probe Discovery (Therapeutics vs. Diagnostics)

As described above, the PI3K/Akt/mTOR signaling pathway is strongly associated with glucose and FDG uptake in cancer cells. That is the reason why FDG is such a great surrogate marker for evolution of therapeutic effects of inhibitors and/or drugs which target the PI3K/AkT/mTOR, signaling pathway. Additionally, as described above, brain tumors should be classified with molecular fingerprints and clinical trials should follow this classification. The PET imaging for cancer diagnosis should also cooperate with this classification. Therefore, it is not unreasonable to propose that the development of molecular therapeutics should be performed in conjunction with the development of molecular diagnostics according to the classification of molecular fingerprints of the disease. However, there is no such discovery platform currently available.

### SUMMARY

A microfluidic system according to an embodiment of the current invention has a pipette system comprising a plurality of pipettes, a microfluidic chip arranged proximate the pipette system, an imaging optical detection system arranged proximate the microfluidic chip, and an image processing system in communication with the imaging optical detection system. The microfluidic chip has a plurality of cell culture chambers defined by a body of the microfluidic chip, each cell culture chamber being in fluid connection with an input channel and an output channel defined by the microfluidic chip. The pipette system is constructed and arranged to at least one of inject fluid through the plurality of pipettes into the plurality of input channels or extract fluid through the plurality of pipettes from the plurality of output channels while the microfluidic system is in operation. A method of automated fluorescent imaging of a plurality of cell cultures includes loading a plurality of cell cultures into a plurality of cell culture chambers of a microfluidic chip, the plurality of cell culture chambers comprising a surface coating of an extracellular matrix material for immobilization of said cell cultures, applying at least one fluorescent probe to the cell cultures and incubating the cell cultures under suitable conditions to promote binding of the probe to a specific target in or on the cells, illuminating the cell cultures to cause the fluorescent probe to emit fluorescent light, and imaging light fluorescing from the cells in each of the plurality of cell culture chambers while the cell cultures remain substantially immobilized in the plurality of cell culture chambers to provide information regarding the specific target in or on the cells.

### BRIEF DESCRIPTION OF THE DRAWINGS

Additional features of this invention are provided in the following detailed description of various embodiments of the invention with reference to the drawings. Furthermore, the above-discussed and other attendant advantages of the present invention will become better understood by reference to the detailed description when taken in conjunction with the accompanying drawings, in which:

FIG. 1 is a schematic illustration of a microfluidic chip for large scale cell culture and assay according to an embodiment of the current invention;

FIG. 2 is a schematic illustration of the relationship between the PI3K signaling pathway and PET probe uptake (the left table shows inhibitors of the PI3K signaling pathway and the right table shows the PET probe and their targets);

FIGS. 3A-3C show PTEN immunofluoresent images in U87 cells and flow-cytometric analysis data for PTEN for U87-PTEN cells FIG. 3(A), U87 cells FIG. 3(B), and the quantitative data corresponding to flow-cytometry for U87-PTEN and U87 cells FIG. 3(C) based on the data of FIGS. 3(A) and 3(B);

FIGS. 4A-4C shows EGFR immunofluorescent images in U87 cells and flow-cytometric data for EGFR (U87-EGFR cells FIG. 4(A), U87 cells FIG. 4(B), and the quantitative data corresponding to flow cytometry for U87-PTEN and U87 cells FIG. 4(C) based on the data of FIGS. 4(A) and 4(B);

FIGS. 5A-5E show EGFRvIII immunofluorescent images in U87 cells and flow-cytometric data for EGFRvIII (U87-EGFRvIII cells in FIG. 5(A), U87 cells in FIG. 5(B), FIG. 5(C) shows average EGFRvIII immunofluorescent intensity of individual cells of U87 cells, FIG. 5(D) shows histograms corresponding to flow cytometry for U87-PTEN and U87 cells based on the data of FIGS. 5(A) and 5(B), and FIG. 5(E) shows two-dimensional plots of DAPI vs. EGFRvIII based on the data of FIGS. 5(A) and 5(B));

FIGS. 6A-E show uptake of glucose analog (2-NBDG) into U87 cells (U87 cells, FIGS. 6(A) and 6(B), U87-PTEN cells, FIGS. 6(C) and 6(D), bright field images, FIGS. 6(A) and 6(C), fluorescent images, FIGS. 6(B) and 6(D), histogram of 2-NBDG uptake in U87 cells, FIG. 6(E)).

FIG. 7 is a schematic illustration of microfluidic system according to an embodiment of the current invention.

FIGS. 8A-8C. (FIG. 8a) Actual view of the microfluidic device for large-scale cell culture and assay; (FIG. 8b) Time lapse micrographs of the U87 cell proliferation in the microchip in a duration of 6 days. (FIG. 8c) quantified proliferations of chip-cultured U87 cells.

FIGS. 9-10. Data collection. (FIG. 9) Data collection resulting in 2-D or 3-D dot plots. (FIG. 10). Data collection resulting in a histogram.

FIGS. 11A-11C. Optimization of antibody concentration for p-S6 staining in a microfluidic cytometry platform.

FIGS. 12A-12F. Robotic pipette for performing large-scale signaling profiling in an automated fashion.

FIG. 13. On chip multiparameter measurement of upstream markers of the PI3K pathway in glioblastoma cells: EGFR, EGFRvIII, and PTEN.

FIG. 14. EGFRvIII, EGFR, PTEN detection comparison of FLOW vs, CHIP—same cell suspensions where run in paralle on chip and by flow.

FIG. 15. Quantitative measurement of PI3K signaling (p-EGFR, p-akt, p-S6) in single cells from molecularly complex heterogeneous glioblastoma samples.

FIG. 16. Characterization of a molecularly heterogenous population on a chip.

FIG. 17. On Chip Detection of “rare cell” within a population.

FIG. 18. Detection of PI3K signaling in a solid clinical glioblastoma sample.

FIG. 19. Detection of low levels of endogenous PTEN.

FIGS. 20A-20F. The SFKs are dasatinib sensitive molecular targets in GBM.

FIGS. 21A-21E. Development of approach to measure effect of mTOR inhibition in glioblastoma patients and demonstration of a resistance promoting feedback loop.

FIGS. 22A-22C. Conceptual summary of the Microfluidic Image Cytometry (MIC) technology.

FIGS. 23A-23C. Optimization of the concentrations of FITC-labeled pS6 antibody for quantifiable ICC.

FIGS. 24A-24C. Dynamic ranges of the MIC technology for single-cell profiling of EGFRvIII, EGFR, PTEN, pAKT and pS6 under the optimal ICC conditions.

FIG. 25. Quantification of PTEN expression and pAKT phosphorylation of two sets of isogenic mouse cell lines, including (i) PTEN ES lines, i.e., p8 (+/−) and CaP8 (−/−), and (ii) MEF lines, i.e. PTEN^{γloxp/loxp}(+/+) and PTEN^{γΔloxp/Δloxp} (−/−).

FIG. 26, Parallel signaling profiling using the MIC technology.

FIGS. 27A-27E. Quantification of pS6 expression levels in individual cell treated with different concentrations of rapamycin.

FIG. 28. Two different view angles of a 3-D scatter plot were utilized to illustrate the cellular heterogeneity of a brain tumor sample analyzed in the MIC-chip.

FIGS. 29A-29F. A robotic pipetting system for performing large-scale signaling profiling in an automated fashion.

FIG. 30. 3-D scatter plots of 12 brain tumor samples analyzed in the MIC-chip 12, revealing dramatically different cellular heterogeneity of individual tumor samples.

FIG. 31. Heretical clustering approach was employed to analyze and quantify cellular heterogeneity of a given patient samples.

FIG. 32. Heatmap of individual cells, where green is low, erd is high expression NS versus SC at 1^{st} passage reveals differential cell populations.

FIGS. 33A-33B. (FIG. 33A) Heatmap of individual cells, where green is low, red is high expression mTOR blocking releases inhibition of pAkt feedback loop, and n is 1. (FIG. 33B) Heatmap of individual cells, where gren is low, red is high expression EGFR blocking reveals EGFR signal may propagate through pAkt.

### DETAILED DESCRIPTION OF VARIOUS EMBODIMENTS

### Micro Total Analysis Systems

In recent years, micro total analysis systems (μTAS) have been of great interest to biological researchers for cellular analysis. A prominent characteristic of μTAS is the capability of constructing highly integrated/functional systems on a microchip. Therefore, many processes that were complicated in conventional cellular analysis could be integrated on stand-alone microchips. This integration resulted in short-time analysis and easy handling for operation. Moreover, these integrated microfluidic systems had advantages such as a reduction in the consumption of cells, reagents, and samples, real-time analysis, and constancy of experimental conditions⁴⁷. As stated above, cellular analysis on an integrated microchip can provide numerous benefits. Thus, cellular analysis on the microchip has been rapidly spreading, for example, to applications of cell sorting⁴⁸, and the introduction of genes into cells.

### Integrated Microfluidics

Poly(dimethylsiloxane) (PDMS)-based integrated microfluidics represent a large scale architecture of fluidic channels that allow for the execution and automation of sequential physical, chemical and biological processes on the same device with digital control of operations^{49,50}. In particular, the elasticity of PDMS materials enable a parallel fabrication of the micron-scale functioning modules, such as valves, pumps and columns⁵¹, that are necessary for sequential operations. In addition, fabrication of intricate devices using this technology requires only relatively simple facilities: the fluidic and control networks are mapped using standard CAD software and transferred onto transparent photomasks. Photolithographic techniques can be used to produce a reusable mold onto which a PDMS resin is poured and cured by baking. Access to the fluidic channels can be achieved by punching holes through the bulk material, and the devices can be readily bonded to glass or silicon substrates, for example. Large arrays of active components, such as valves and pumps, can be created by stacking multiple, individually fabricated layers. When pressurized with air or inert gases, a channel on the control layer that crosses a channel on the flow layer is deflected, sealing the flow channel and stopping fluid movement. This method of valve operation also constitutes the binary switches (e.g., open or closed) of the microfluidics chip. Using this simple fabrication technology, our joint research team has demonstrated devices of remarkable diversity, including microfluidic devices with chemical reaction circuits (PCT Int. Appl. 2006, WO 2006071470 and U.S. Patent Application 2007, No. 60/876,525), an integrated microfluidic blood sampler for mice (UCLA Case No. 2005-659-1), a microfluidic platform for high sensitivity quantification of radioisotope concentrations (U.S. Provisional Patent Application, UCLA Case No. 2006-388-1) and a microfluidic platform for cell culture and assay (U.S. Provisional Patent Application No. 60/876,525 filed Dec. 22, 2006), the entire contented of which are incorporated herein by reference.

### Microscope-Based Cytometry

Single-cell measurements can reveal information obscured in population averages. For example, studies of variation in gene expression in individual Escherichia coli and S. cerevisiae^{52,53} cells have shown that only a fraction of cell-to-cell variation in the expression of reporter genes results from stochastic fluctuations in the workings of the gene expression machinery⁵⁴⁻⁵⁶, and have identified other processes and genes that account for and control the bulk of the variation⁵⁴.

One means to collect single-cell data is flow cytometry, whose development began in the 1930s. Modern instruments are powerful but (i) cannot interrogate individual cells repeatedly to produce time series for each cell, (ii) cannot collect a great deal of light, owing to both the short time (typically microseconds) that the cell passes the detector and to the numerical aperture of the objective, which often collects less than 10% of the emitted light, and (iii) typically do not capture images of the cells, making it difficult to analyze cell shape, size and intracellular localization of fluorescence. Some recent work has attempted to address the last two limitations by integrating the fluorescence signal over longer times and capturing cell images with custom-built charge-coupled device (CCD) detectors⁵⁷. Optical microscopy can compensate for some limitations of flow cytometry by providing abilities to revisit individual cells over time, collect emitted light for long times and capture cell images with high resolution. Automation by computer-aided cell tracking and image analysis began in the 1960s and can permit generation of some such data with high throughput⁵⁷⁻⁶³. However, such approaches of single cell tracking have limitations in practical applications, such as low throughput and/or poor cell viability.

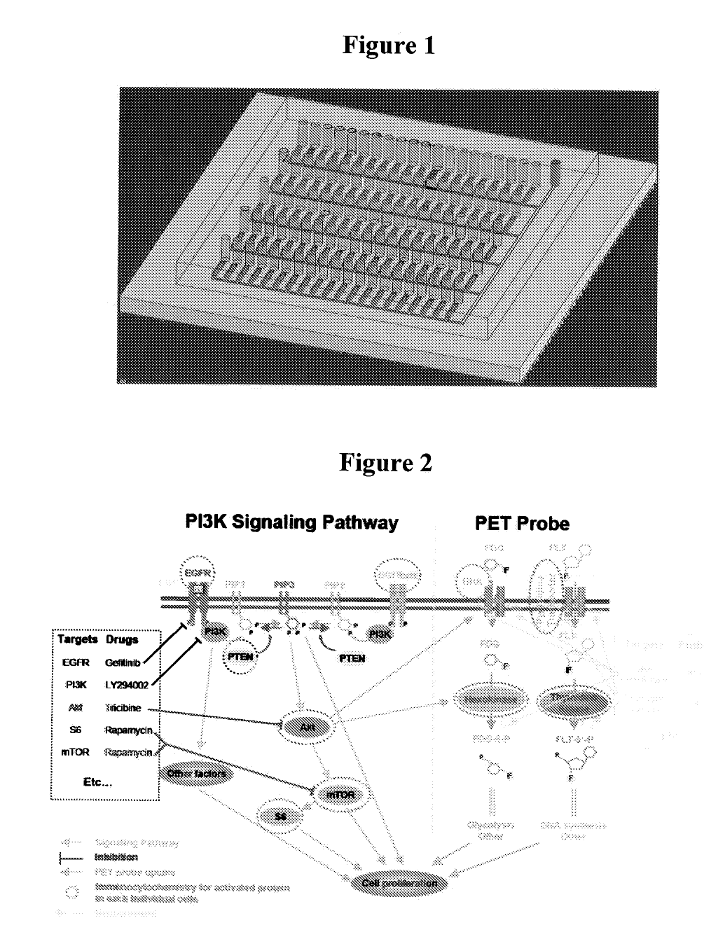

According to an embodiment of the current invention, new PDMS-based microfluidic devices (see FIG. 1) can allow one to perform both drug screening and PET probe discovery based on monitoring the PI3K/Atk/mTOR signaling pathway and imaging probe uptake in conjunction with fluorescent microscopy and an embedded radiodetector, respectively. (See also PCT/US2007/009705, filed Apr. 20, 2007.) Glioblastoma cell line and genetically manipulated glioblastoma including U87, U87-PTEN, U87-EGFR and U87 EGFRvIII were analyzed in a microfluidic device accord to an embodiment of the current invention. Intrinsic advantages of such microfluidic systems can include sample and/or reagent economy, high throughput operation, experimental fidelity, scalability, flexibility and digitalized controllability. Such a microfluidic platform according to some embodiments of the current invention can be utilized for high throughput screening of drug and imaging probe candidates. By including a robotic pipetting system according to some embodiments of the current invention, it is feasible to perform parallel screening of 1000 to 10000 cell cultures with a stand-alone device and to still have advantages of sample economy and quantitative/systems readout at the single-cell level. Between 10 to 1596 chambers have been found to be suitable according to some embodiments of the current invention.

### High Throughput Drug Screening

This microfluidic platform according to some embodiments of the current invention has the potential to significantly enhance the throughput of cell analysis with microscope-based cytometry. This device can enable one to analyze the drug effects on PI3K signaling pathways in individual cancer cells and assess PET probe uptake at the same time (FIG. 2).

### Smaller Size and Less Cost

An intrinsic advantage of drug screening and PET probe discovery in a microfluidic device according to some embodiments of the current invention is that we can reduce the volume of medium, antibodies, PET tracers, and so on used. Volumes as low as about 200 pL per microchamber have been found to be suitable according to some embodiments of the current invention.

### Monitoring Signaling Pathway

In an embodiment of the current invention, signatures of the PI3K/Akt/mTOR signaling pathway are stained with immunofluorescent methods. We can then analyze drug effects on PI3K signaling pathways with a microscope-based cytometry system according to an embodiment of the current invention.

### Classification for Glioblastoma by Molecular Fingerprint in Individual Cells

This device allows us to classify glioblastoma of patients with a molecular fingerprint analysis for individual cells. According to this classification, we can choose effective drugs for each glioblastoma patient.

### PET Probe Discovery in Conjunction with Molecular Fingerprinting in Individual Cells

At the same time, we can assess and choose an effective PET tracer for each patient with the microfluidic system according to an embodiment of the current invention. According to sonic embodiments of the current invention, we can develop the various PET tracers for each molecular event in cancer cells.

### High Throughput Mammalian Cell Culture in Microchips

Dr. Luke Lee's group at UC Berkeley presented a high aspect ratio microfluidic device for culturing cells inside an array of microchambers with continuous perfusion of medium. The device was designed to provide a potential tool for cost-effective and automated cell culture. The single unit of the array consists of a circular microfluidic chamber 40 μm in height surrounded by multiple narrow perfusion channels 2 μm in height. The high aspect ratio (˜20) between the microchamber and the perfusion channels can offer advantages such as localization of the cells inside the microchamber as well as creating a uniform microenvironment for cell growth. Finite element methods were used to simulate flow profile and mass transfer of the device. Human carcinoma (HeLa) cells were cultured inside the device with continuous perfusion of medium at 37° C. and were grown to confluence.

### Drug Screening with Microchips

High-throughout measurement of ion-channel activity by patch damping is of considerable interest in drug discovery as a tool to characterize therapeutic molecules. Microsystems that combine high throughput with small reagent volumes have led to commercial microscale patch-clamp devices. In these devices, ion-channel recording is typically achieved by placing cells on a micrometer-sized aperture in a membrane that separates two electrodes^{64,65}. By guiding cells onto apertures using microfluidic paths, it is possible to reduce the otherwise labor-intensive micromanipulations needed to locate cells at recording sites and to present the cell with successive stimuli⁶⁶. Obtaining the high-electrical-resistance seals necessary for high quality ion-channel recording (˜10⁹Ω) is technically challenging on both a macro- and a microscale, and microsystems have been more successful in meeting the throughput challenge.

### Single-Cell Analysis

In both conventional studies and microsystems, the analysis of single cells has typically been performed using image-based techniques and intracellular fluorescent probes (such as those that measure calcium flux⁶⁷). However, the ability of integrated microfluidics to accurately manipulate, handle and analyze very small volumes has opened up new opportunities for analysis of intracellular constituents. A microfluidic device with integrated pneumatic valves capable of isolating single cells and then lysing them using a chemical lysis buffer has been shown to be capable of extracting and recovering messenger RNA from a single cell⁶⁸. A similar device that also integrates electrophoretic separation can analyze amino acids from the lysed contents of a single cell⁶⁹. Single cell analysis by electrophoretic separation, but with electrokinetic flowdriven cell loading, docking and lysis have also been demonstrated⁷⁰.

### Molecular Fingerprint for Glioblastoma

Using an immunohistochemical analysis applied to a tissue microarray, Dr. Mische's group performed hierarchical clustering and multidimensional scaling, as well as univariate and multivariate analyses, to dissect the PI3K pathway in vivo¹⁷. The results provide the first dissection of the PI3K pathway in glioblastoma in vivo and suggest an approach to stratifying patients for targeted kinase inhibitor therapy. Additionally, DNA-microarray analysis is most useful when it can be integrated with clinical, imaging and histological data. Substantial effort is required to develop appropriate databases that contain key clinical information, including patient characteristics such as age and sex. Brain imaging is routinely undertaken and images are housed in a central database. Histological photomicrographs document cellular morphology, and clinical data are entered in real time through wireless input devices to ensure accurate and up-to-date information. Biopsy material is preserved for future analyses, linked to clinical data and used to extract RNA for large-scale expression analysis using microarrays⁷¹.

### Microscope-Based Cytometry

During the past 20 years, a good deal of research-directed automated microscope-based cytometry outside of clinical and pharmaceutical applications has relied on two commercial software packages, Metamorph (Molecular Devices Corporation) and ImagePro (Media Cybernetics, Inc.), to operate the microscopes, collect the data and analyze them. These packages, often used together with more general purpose analysis programs, such as Matlab (The Mathworks, Inc.) and Labview (National Instruments Corporation), probably constitute the state of the art in commercial software used for these purposes. Likewise, open-source projects can provide valuable tools for image analysis. Examples include the Open Microscopy Environment (OME), which provides file formats and metadata standards for microscope images14, Image J, a Java-based package of microscope image analysis tools15, and CellProfiler16.

### Microchips in an Embodiment of the Current Invention

The prototypical microchips for drug screening and PET probe discovery (FIG. 1) based on the PDMS-based microfluidic system were designed and fabricated. The channel parameter is 300 μm (W)×2000 μm (L)×200 mm (H). The PDMS chip was tightly attached on a glass slide with oxygen plasma treatment for 30 seconds. Extracellular matrix components (e.g., fibronectin (FN), laminin, Matrigel and RGD peptide) can be coated onto the surfaces of all channels for immobilization of cells.

In some embodiments, microfluidic chips with dimensions as small as the following has been used, Chamber dimension: (100 μm (l)×100 μm (w)×20 μm (h), with a volume of 200 pL. Commonly used microfluidic chips to date have the following dimensions. Chamber dimension: (3000 μm (l)×500 μm (w)×100 μm (h), with a volume of 150 nL. In other examples, chambers as large as the following have been used according to embodiments of the current invention. Chamber dimension: (6000 μm (l)×2000 μm (w)×200 μm (h), with a volume of 2.4 μL.

### Glioblastoma Cells

U87 human glioblastoma cell line and stably growing cells from primary glioblastomas from patients treated at UCLA for brain tumors (NS 107, NS 117, NS 146) were first selected for the proof-of-concept trial. U87 cells were modified with retroviruses which have PTEN, EGFR or EGFRvIII driven by the CMV promoter to construct model cell lines for glioblastoma patients (U87-PTEN, U87-EGFR and U87-EGFRvIII).

### Immunofluorescent Staining for Cells in Microchips

Fibronectin (FN), which is the extracellular matrix, was coated onto the surfaces of all channels for cell adhesion. 8.0 μl of FN with a concentration of 250 μg/ml was introduced into each channel and incubated at 37° C. for 30 min. The mixture of suspended U87 cell lines obtained from regular cell culture setting was introduced into the FN-coated channels and kept in an incubator for 30 min. Then the channel was rinsed with 100 μL of cell culture medium (Dulbecco's Modified Eagle Medium+10% Bovine Calf Serum+1% Penicillin-streptomycin+1% L-glutamine). After being left overnight, cells in the channels were fixed with 4% paraformaldehyde for 15 min at room temperature. After 15 min, each channel was rinsed with PBS. To prevent non-specific binding of antibody onto the surface of each channel, blocking solution (10% normal goat serum, 0.1% Triton X-100 and 0.1% N-Dodecyl-β-D-maltoside in PBS) was loaded into each channel and incubated for 30 min. To visualize the PI3K/Akt/mTOR. signaling pathway, antibodies were used; mouse anti-hPTEN (Cascade Bioscience) at 100 μg/mL, mouse anti-hEGFR (Zymed Laboratories) at 15 μg/mL, anti-hEGFRvIII (Dako) 87 μg/mL. All antibodies were labeled with mouse IgG Labeling kits (invitrogen). Those antibodies were loaded into each channel and incubated at room temperature for 30˜60 min. After immunostaining, DAPI was loaded into each channel for nuclear staining. After completion of all staining, microchips were monitored with Nikon TE-2000 microscopy, and the images were analyzed with Metamorph® (Molecular Devices) imaging software.

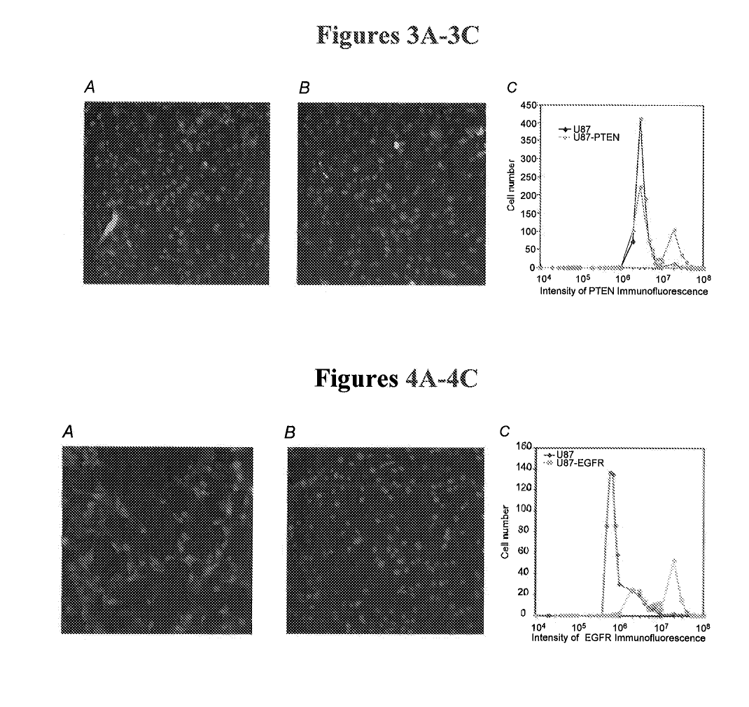

FIG. 3 shows immunofluorescent images of PTEN expression in U87-PTEN (FIG. 3A) and U87 cells (FIG. 3B). Based on these pictures, the quantitative data were analyzed like low cytometry generated with Metamorph (FIG. 3C). Using DAPI staining as the indicator of cells, we can know which cells have positive or negative signals of immunofluorescent staining, and monitor intensity of individual cells. In the case of U87-PTEN, two peaks at different intensity of PTEN immunofluorescent were observed. The cells at higher intensity show the PTEN positive cells by immunofluorescent staining, and the cells at lower intensity show the PTEN negative cells.

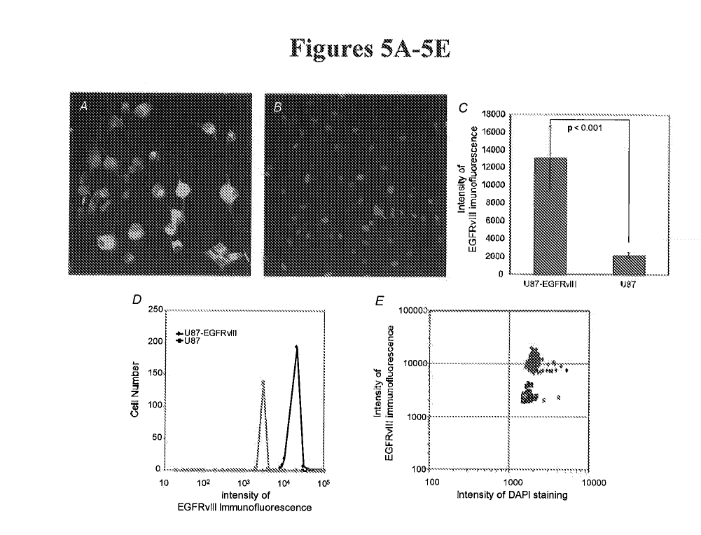

In the cases of EGFR and EGFRvIII, we can also quantitatively analyze population of EGFR negative/positive cells and EGFR immunofluorescent intensity of individual cells (FIGS. 4 and 5). We confirm our methods could be applied to analyze and classify glioblastoma. In the case of FIG. 5, we show the capability of our microscope-based cytometry for 2-Dimensional analysis for double staining. It enables us to get more information, such as identification for individual cells and monitoring signaling pathways at single-cell level, from our immunofluorescent images.

### Monitoring Glucose Uptake in U87 Cells

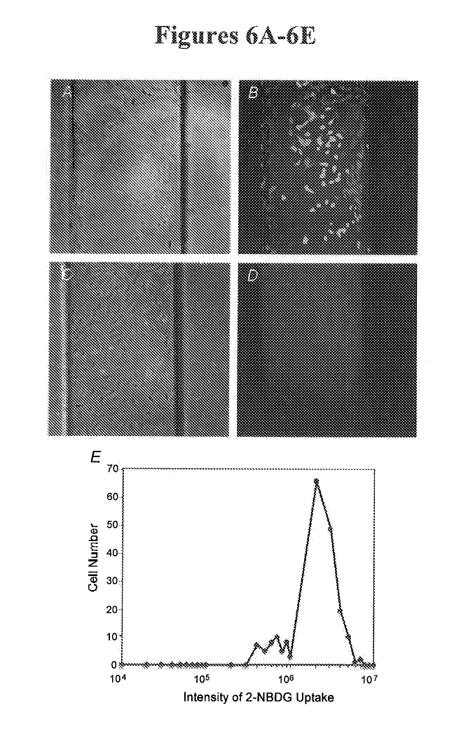

To monitor glucose uptake in U87 cells, a fluorescent deoxyglucose (2-[N-(7-nitrobenz-2-oxa-1,3-diaxol-4-yl)amino]-2-deoxyglucose; 2-NBDG)^{72,73} was used and monitored the 2-NBDG uptake by U87 and U87-PTEN cells (FIG. 6). After cells were loaded into a chip, culture medium was washed with Hanks' balanced salt solution (HBSS) with CaCl₂. Then, 2-NBDG in HBSS was loaded into a cell chamber and incubated for 20 min in an incubator. In the case of U87 cells, the signals of 2-NBDG were observed as shown in FIG. 6B. In U87-PTEN cells, the fluorescent signal by 2-NBDG uptake could not be detected (FIG. 6D). Histograms of 2-NBDG uptake in U87 cells are shown in FIG. 6E. This result shows overexpression of PTEN down-regulated the localization of Glut to cell membrane and hexokinase activity as well as the PI3K/Akt/mTOR signaling pathway. According this result, monitoring both the PI3K signaling pathway and glucose uptake should be in an important place to screen drugs for PI3K/Akrt/mTOR signaling pathway and to discovery new PET probes.

We have demonstrated that this type of microfluidic device according to an embodiment of the current invention can be utilized for glioblastoma and analysis including patient samples. The microfluidic device according to some embodiments of the current invention can provide a platform to monitor individual cells. A microfluidic device according to some embodiments of the current invention can be used to provide cell analysis. A microfluidic system according to some embodiments of the current invention can also be used for high throughput drug screening. Cost reductions can be achieved for cell analysis according to some embodiments of the current invention. In addition, a microfluidic system according to some embodiments of the current invention can be used for PET probe discovery.

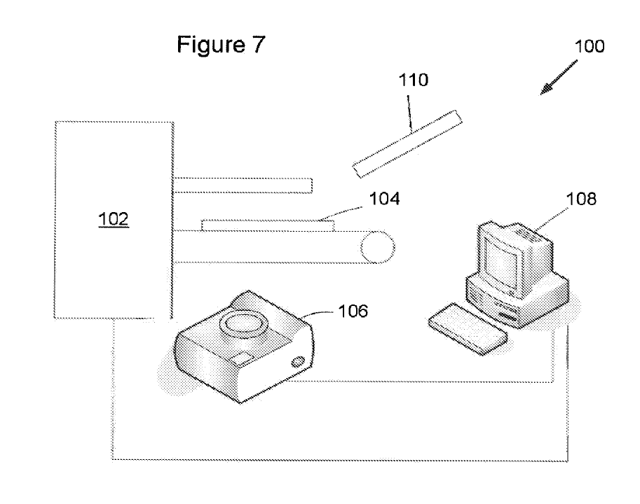

A microfluidic system 100 according to an embodiment of the current invention is illustrated schematically in FIG. 7. The microfluidic system 100 has a pipette system 102, a microfluidic chip 104 arranged proximate the pipette system 102, an imaging optical detection system 106 arranged proximate the microfluidic chip 104, and an image processing system 108 in communication with the imaging optical detection system 106. The pipette system 102 can include a plurality of pipettes. The microfluidic system 100 can also include an illumination system 100 according to an embodiment of the current invention that is constructed and arranged to have an optical path to the microfluidic chip 104. The illumination system 110 is suitable to illuminate cells cultured in said cell culture chambers while in operation. In some embodiments of the current invention, the illumination system may be a white light or broad spectrum illumination system, polychromatic illumination system having multiple spectral lines and/or a monochromatic illumination system, such as a laser.

The microfluidic chip 104 has a plurality of cell culture chambers defined by a body of said microfluidic chip 104. Each cell culture chamber is in fluid connection with an input channel and an output channel defined by the body of the microfluidic chip. The microfluidic chip 104 can be a PDMS-based chip such as that described above in reference to FIG. 1.

The pipette system 102 is constructed and arranged to at least one of inject fluid through the plurality of pipettes into the plurality of input channels or extract fluid through the plurality of pipettes from the plurality of output channels while the microfluidic system 100 is in operation. For example, the pipette system 102 can inject a fluid containing cells to be cultured, can inject culture media and/or drugs under investigation, and can inject other biomarkers, etc. according to some embodiments of the invention. The pipette system 102 may also extract fluid from the output channels of the microfluidic chip 104 either with the same plurality of pipettes or with another plurality of pipettes, for example for cell perfusion, etc.

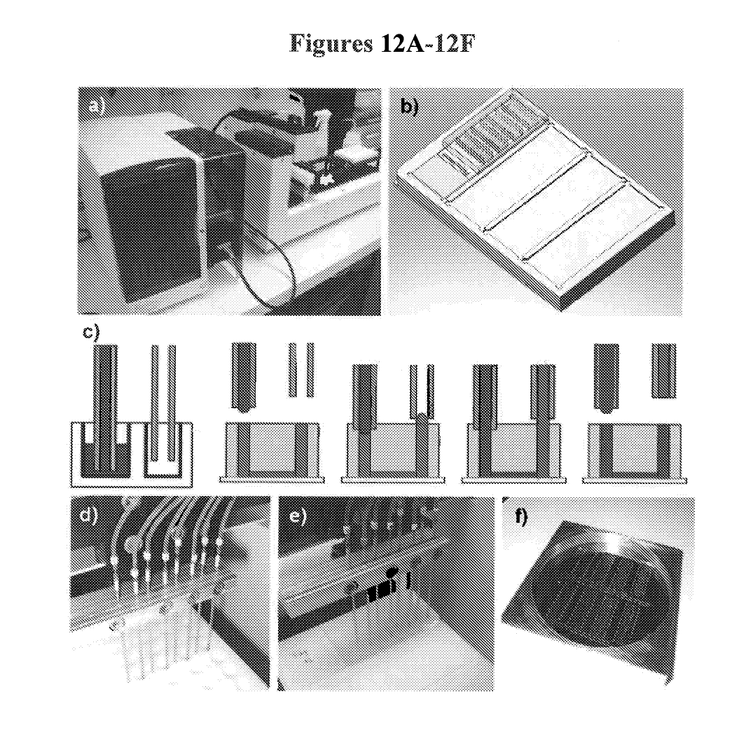

The pipette system 102 can also be a robotic pipette (See FIGS. 12A-12F). However, the scope of the current invention is not limited to only robotic pipette systems. The pipette system 102 can also be a manual or a semi-automated according to other embodiments of the current invention. However, automated operation from initial cell culture/media exchange to immunostaining can permit large-scale cell culture/assay to be carried out for high-throughput signaling pathway profiling.

A user-friendly interface that can include a chip holder (See FIGS. 12A-12F) and a pipette tip array (See FIGS. 12A-12F). A robotic pipette can be designed to handle 96, 396 and/or 1536-well plates, for example. One can take advantage of current standard equipment such as using a chip holder that adopts the dimension of current well plate platforms. Thus, two plate holders can be directly mounted for use with the robotic system without further modification. Currently, one chip holder can accommodate four microfluidic chips, and there are 40 cell culture/assay chambers in a microfluidic chip. The location of each inlet and outlet holes of cell culture chambers can be registered to a specific well location of a 1536 well plate.

The microfluidic system according to some embodiments of the invention can include manual, semi-automated and/or automated operation of the following:
1. Cell culture sample preparation, including cell loading, cell culture in incubator and media exchange for cell maintenance.
2. Immunocytochemistry: including cell fixation, permeabilization, and immunostaining.

The microfluidic system 100 can provide, according to some embodiments of the current invention, a system for (i) large scale cell culture for high-throughput screening, (ii) microfluidic cytometry for quantification of biomolecules with single-cell precision, (iii) signaling pathway network profiling in conjunction with cancer diagnosis and therapeutic stratification, (iv) dynamic protein quantification as an alternative to Western Blot, and other broader applications for quantitative proteomic analysis in cells.

Potential applications of the microfluidic system 100 can include, but are not limited to, the following:
1. An alternative technology for flow cytometry can include advantages of (i) Low cost, (ii) small patient sample/reagent consumption (iii) suitability for both suspension and adherent cells, (iv) a microfluidic environment (with semi- or fully-operation) that can provide experimental fidelity, (v) original data of individual cells can be tracked by looking at the fluorescence images, (vi) superior measurement dynamic range capable of capturing tiny changes in the culture systems, and (vii) single-cell precision allows tackling tumor heterogeneity issues.
2. An alternative technology for Western Blot with advantages that can include (i) low cost, (ii) small patient sample/reagent consumption, (iii) a microfluidic environment (with semi- or fully-automated operation) that can provide experimental fidelity, (iv) single-cell precision can allow detecting protein deletion/down-regulation in tumor samples, (v) large-scale analysis in parallel, (vi) original data of individual cells can be tracked by looking at the fluorescence images, (vii) superior measurement dynamic range, and (viii) capability of capturing tiny changes in the culture systems.
3. Pinpoint alterations in single cells and cell subsets. For example to provide answers to the following question: What signaling mechanisms are active in cancer cells that return during patient relapse, in pre-metastatic cells and during the earliest stages of transformation?
4. One can look not only at ‘pathways’, but at the network as a whole, e.g., to answer the following questions: What are the ‘on target’ and ‘off target’ effects of drugs?
5. Identify and track cancer stem cells. For example: is there a phenotypically distinct subset of cells that is not killed by therapy and that mediates relapse?
6. Identify targets for drug discovery. For example: What signaling mechanisms enable cancer cells to resist a particular chemotherapy?
7. Choose an optimal therapy, For example: Do patients that respond to a particular cancer therapy have similar signaling profiles?
8. Monitor anticancer therapies, For example: Can signaling profiles be used as biomarkers of therapeutic response or side effects?
9. Detect cancer earlier. For example: Can signaling profiles of circulating cancer cells, or of immune system cells, be used to detect cancer at early stages?
10. Understand mechanisms of cell-cell and cancer-cell-host interactions. For example: How do cancer cells interact with and alter the host microenvironment or immune system?

Signaling network profiling according to some embodiments of the current invention can provide the following:
1. Unlike serum biomarker analysis (one major approach for cancer diagnosis), studying a signaling network can help one to better understand disease and therapeutic stratification targeting at molecular lesions.
2. Quantified signaling network profiling (on multiple signaling nodes) can be a much more precise cancer diagnostic technology (in staging and diagnosis) than conventional pathology approaches based on tissue morphology, western blot and immunohistochemistry (IHC).
3. Biopsy samples can be obtained routinely in clinics. Of course, an appropriate tissue processing technology should be used.
4. Quantified signaling network profiling allows multi-dimension systems-biology study (perturbation/measurement along a time course), providing a more dynamic understanding of how information is processed by the system.
5. Quantified signaling network profiling can be utilized for evaluation of therapeutic effect of new drug which can accelerate clinical trial (phases II and III).
6. Chip-based, study may potentially give a platform for performing in vitro therapy (treat patient tumor tissue samples in the device and monitoring their therapeutic responses in the chips), that predicts therapeutic effects before patients are treated. By using this approach, multiple drugs or cocktail therapeutic approaches can be tested and evaluated in very short period (e.g., 48 hr) without risk.

### Example A

### Assay Example

1) Sample preparation, including cell loading, cell culture in an incubator and media exchange for cell maintenance.

2) Immunocytochemistry, including cell fixation, permeabilization, and immunostaining.
Multiple signaling nodes including EGFR, EGFvIII, PTEN, pAkt, pmTOR, pS6, and the proliferation marker Ki67, involved in PI3K-Akt-mTOR signaling network can be measured in glioblastoma system.
Multiple signaling nodes including EGFR, ErbB2, PTEN, pAkt, pmTOR, pS6, and the proliferation marker Ki67, involved in PI3K-Akt-mTOR signaling network can be measured in breast cancer system.

### Growth Curve of the Living Cell can be Monitored as the Follows:

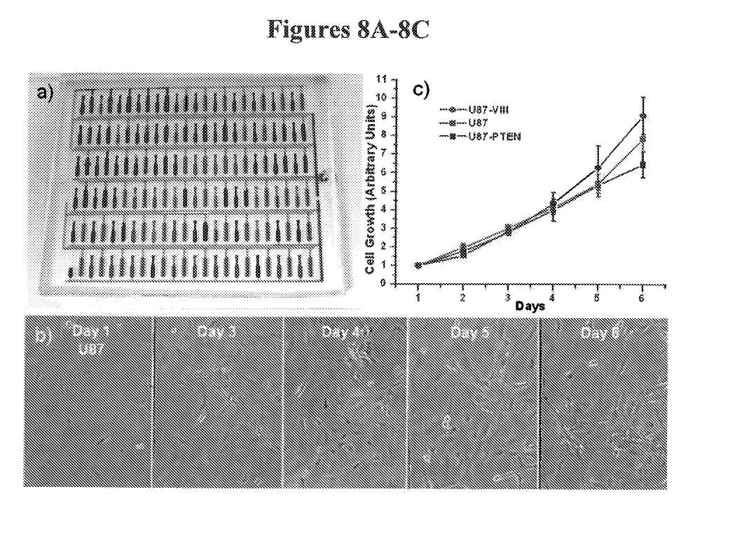

The cell culture/assay chip shown in FIG. 8 composed of two types of microchannels responsible for (i) performing 72 cell cultures and assays in parallel and (ii) moisturizing the adjacent cell culture chambers (preventing media evaporation). Meanwhile, we have devoted a significant amount of efforts to study surface modification of the microchannels to ensure cell viability over a period of 7 days. A semi-automated pipette with 12 pipette tips was employed for loading cells, changing culture media and introducing fixation, permeabilization and immunostaining reagents. Constant media exchange allows on-chip cell culture for six days or longer time. A number of genetically manipulated glioblastoma cell lines (i.e., U87, U87-PTEN, U87-EGFR, U87 EGFRvIII and U87-EGFRvIII/PTEN) have been successfully cultured in the devices with reproducible growth rates compatible with those obtained in the macroscopic setting.

FIG. 8A is an actual view of the microfluidic device for large-scale cell culture and assay, in which two types of fluidic channels have been loaded with food dyes to help visualize the different functions of the microfluidic chip: (red) moisturizing channels and (green) cell culture, channels. (FIG. 8B) Time lapse micrographs of the U87 cell proliferation in the microchip in a duration of 6 days. (FIG. 8C) The proliferations of chip-cultured U87 cells were quantified by monitoring the number of U87 cells inside the cell culture chambers over time.

### Example B

### Data Collection

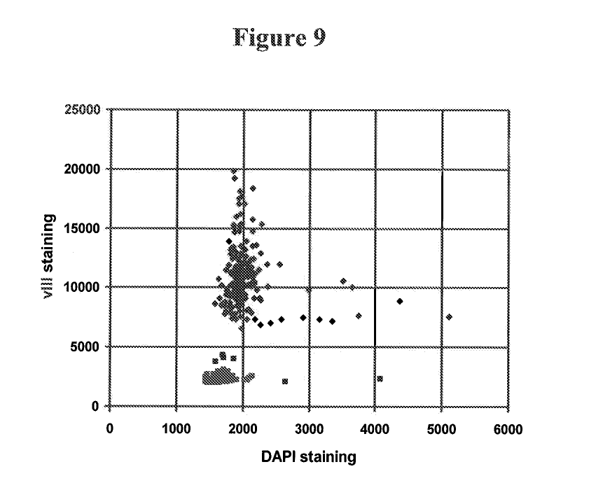

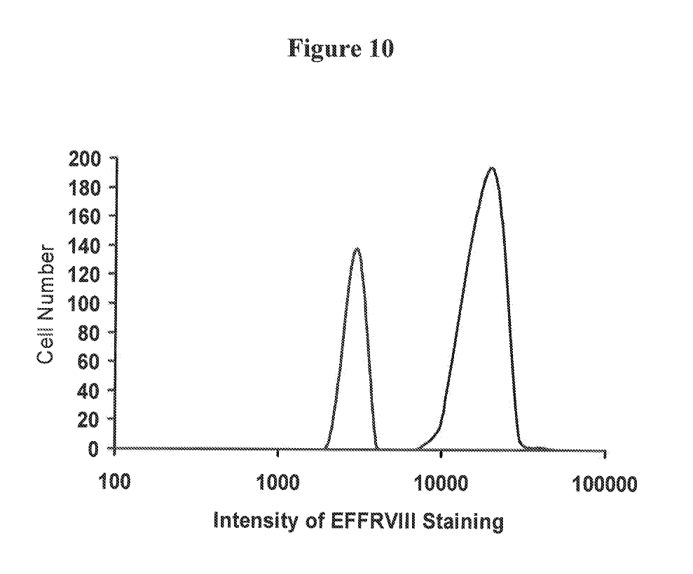

Data obtained from methods according to some embodiments of the current invention may be in the form, for example, of 2-D or 3-D dot plots (X axis—intensity of one signaling node (in this case, EGFRvIII) and Y axis—intensity of the other signaling node (in this case DAPI)) for 3-D dot plots Z axis—intensity of a third signaling node (FIG. 9). In another embodiment (FIG. 10), the data may be in the form of histograms (X axis—intensity of one signaling node (in this case, EGFRvIII) and Y axis—cell number).

### Protocol for Cell Culture for Glioblastoma in a Chip

### Materials:

24-channel poly-L-Lysine-coated cell culture chips
Cell culture medium: 500 mL Dulbecco's Modified Eagle Medium
50 mL Fetal Bovine Serum
5 mL Pen-strep/L-glutamine

### Cell Lines and Isogenetic Cells:

U87
U87-PTEN
U87-EGFR
U87-EGFRvIII
U87-EGFRvIII/PTEN

### Automated Pipettes:

Multichannel (From Matrix Technologies Corporation, two suggested pipettes with Item No. 2239 and 2139)
Singlechannel (From Matrix Technologies Corporation, pipette Item No. 1029)

### Instruments;

Nikon-TE2000 microscope

### Procedure:

### Cell Loading:

1. Sterilize the chip in a biosafety hood under UV light irradiation for 15 min.
2. Wash each channel twice with cell culture medium (2 μL for each time); loading speed for pipette is 6 μL/s. Use pipette tip (connected with vacuum) to remove the exhaust solution.
3. Load 2 μL 30% FBS in cell culture medium into each channel.
4. Put the chip into a Petri dish and drop 0.5 mL sterilized water into the dish.
5. Keep the dish in the incubator for 24 hours.
6. Take the dish out of the incubator. Wash each channel twice with 2 μL of cell culture medium; loading speed for pipette is 6 μL/s. Use pipette tip (connected with vacuum) to remove the exhaust solution.
7. Load 2 μL of 8×10⁵ cells/mL cell suspension to each channel with pipette loading speed 0.55 μL/s.
8. Put the chip in an incubator (37° C. and 5% CO₂)
9. Keep the chip in an incubator for overnight to ensure good cell attachment.
10. Change medium for the cells every 12 hours by loading 2 μL/channel fresh medium with loading speed 0.55 μL/s.
11. Take 10 pictures for each channel to monitor cells with Nikon-TE2000 microscope everyday.
12. Culture the cells inside the chips for 6 days.
   Tips: Bubbles sometimes may be generated in a chip. A drop of culture medium at the inlets helps to decrease the chance to generate the bubbles in a chip.

### Protocol for PDMS Microfluidic Chip Fabrication

Materials: Sylgard 184 PDMS (Dow Corning Corp. Catalog No. 4019862)
Silicon Wafer with defined photolithography design (Silicon Quest)
Petri Dish (VWR LABshop Catalog No. 25384-302)
Poly-L-Lysine Microscope Slides (Polysciences, Inc. Catalog No. 22247)
Micro Slides (Pre-Cleaned 75×50 mm Corning)
Toluene (Sigma, Catalog No. 244511)

### Procedure:

Microfluidic Mold Preparation
   1. Place silicon wafer template into an aluminum foil covered 10 cm petri dish
   2. Use stir stick to mix 33 g of 10:1 (ratio reagent A:B) Sylgard PDMS in a clean mixing cup
   3. Pour the mixed PDMS into the petri dish over the silicon wafer
   4. Vacuum pump PDMS/dish for 40 minutes to remove all air
   5. Put petri dish into oven and incubate at 80° C. for 30 minutes
   6. Remove petri dish from oven and let it cool at room temperature for 30 minutes
   7. Gently lift away aluminum foil/wafer/PDMS mold from petri dish
   8. Gently cut ‘X’ into aluminum foil/PDMS on underside of wafer using razor blade and peel away to expose underside of silicon water
   9. Gently press edges of PDMS mold to release silicon wafer from mold
   10. Cut the PDMS mold into chips and punch holes for each chip (alternatively, holes can be punched first)
   11. Clean each mold by drawing adhesive tape across microchannels and chip top twice to remove dust and PDMS debris

### Chip Fabrication

1. Use glass stir stick to mix 6 g of 5:1 (ratio reagent A:B) Sylgard PDMS in a clean mixing cup
2. Dissolve 5 g mixed PDMS mixture into glass beaker of 20 mL toluene and mix well with glass stir stick
3. Spin-coat the PDMS toluene solution on a Micro Slide (75×50 mm) at 4000 rpm, 30 s
4. Put the fluidic mold (above) onto the Micro Slide to so a thin layer of PDMS glue will coat microchannel side of PDMS fluidic mold
5. Place the fluidic mold onto a commercialized poly-L-lysine slide
6. Put the microfluidic chip into a vacuum oven and incubate at 80° C. for 24 hours

### Metamorph Microscope Protocol

### Materials:

MetaMorph (Premier version): Molecular Devices
Microscope: Nikon TE2000S (Epifluorescent microscope)
Samples

### Operation:

1. Turn on the microscope in the following operation order: (i) bright-field light source (Light 1), (ii) fluorescence light source—Xe lamp (Light 2), (iii) CCD monitor, (iv) camera, and (v) computer (if it is not on).

*** Please make sure that Xe lamp is completely cooled prior to turning on the microscope, and that Xe lamp remains on at least 30 minutes during every use.
2. Place slide on the stage.
3. Open Metamorph program (by clicking the icon on the desktop), and drop down the acquire menu and select “Acquire”.

*** There are two options for viewing images:
1) At the eyepiece, the PORT knob (on the right-hand side of the camera) should be turned to “EYE”
2) On the monitor, the PORT knob is turned to “SIDE”. This is also the option for CCD camera use.
4. Select an adequate filter at Setting in the “Acquire Dialog Window” or using the option panel. Different options, including brightfield, DAPI, FITC, TRITC, Cy5 and Li-cor will be decided. Operation parameters, including digitizer, gain and exposure time will be determined at this point.

*** a) We recommend to use “1 MHz” for “Digitizer” and “3 (4×)” for “Gain” when taking fluorescence images.
b) Adjust the exposure time to obtain a fluorescent intensity is located in the range between 2000 (Pixel) and 65535 (anywhere between 0.1-10 seconds is common)
For brightfield exposure time should be ca. 50 ms.
For DAPI, exposure time should be ca. 10 ms.
For FITC, exposure time should be ca. 100-500 ms.
For TRITC, exposure time should be ca. 200-2000 ms.
For Cy5, exposure time should be ca. 500-2000 ms.
For Li-cor, exposure time should be ca. 1-10 sec.
5. Click “Show live” to see the image. Focus the image using the focus wheel and move the slide using position control stick on the “ASI” control box.
6. Click. “Acquire” to acquire the desired image.

*** Before taking the fluorescence image, turn off the brightfield light with the button on the Nikon control box.
7. Drop down the “File” menu and select “Save as” to save images.
8. After taking pictures, close the microscope in the following operation order: (i) the Metamorph software, (ii) the fluorescence light source, (iii) bright light source (iv) CCD monitor and the camera, and (v) the computer.

### Multiple Cell Scoring by MetaMorph:

1. Open a set of images saved from above (DAPI and FITC, TRITC, CY5 or Li-COR)
2. Click “Multiple Cell Scoring” under the menu “Apps”
3. Click “All Nuclei” and activate image through “W1 source image”
4. Set parameters for “Approximate min width” and “Approximate max width” according to size of each nucleus (from the DAPI image)
5. Set a parameter for “Intensity above local background” according to the minimum pixel difference between signal and background (from the DAPI image)
6. Click “FITC (or TRITC, CY5, Li-COR)” and activate an image through “W2 source image”
7. Set parameters for “Approximate min width” and “Approximate max width” according to size of each cytoplasmic area (from the FITC image)
8. Set a parameter for “Intensity above local background” according to the minimum pixel difference between signal (each cytoplasmic area) and background (from the FITC image)
9. Click “Apply”
10. Click “Open log”
11. Click “Log data”—the data will automatically export to Excel.
12. “Integrated intensity” or “Average intensity” values can be used for analysis (Histogram, or Scatter Plot like FACS analysis)

### Protocol for Immunocytochemistry for Glioblastoma in a Chip

### Materials:

24-channel poly-L-Lysine-coated cell culture chips
Cell culture medium:
500 mL Dulbecco's Modified Eagle Medium (Invitrogen, Catalog No.: 11965-118)
50 mL Fetal Bovine Serum (Omega Scientific, Cat # FB-01)
5 mL Pen-strep/L-glutamine (Invitrogen-Gibco)
4% Paraformaldehyde (Electron Microscope Sciences)
Triton X-100 (Fluka)
Methanol
Phosphate Buffered Saline (PBS) (Mediatech, Inc, Lot No. 21040136)
Normal Goat Serum (NGS) (Fisher)
Bovine Serum Albumin (Sigma)
N-Dodecyl-β-D-maltoside (NDBM) (Pierce, Cat #89902,89903)
Zenon Mouse IgG Labeling Kits (Invitrogen/Molecular Probes)
HiLyte750 Labeling Kit —NH₂ (Dojindo, LK16-10)
DAPI nuclear staining reagent (Molecular Probes, Lot 35033A)
Antibodies (*Please Aliquot the Antibody when you receive it)

### Cell Lines and Isogenetic Cells:

U87
U87-PTEN
U87-EGFR
U87-EGFRvIII
U87-EGFRvIII/PTEN

### Automated Pipettes:

Multichannel (From Matrix Technologies Corporation, two suggested pipettes with Item No. 2239 and 2139).
Singlechannel (From Matrix Technologies Corporation, pipette Item No. 1029)

### Procedure:

### Cell Loading:

1. Sterilize the chip in a biosafety hood under UV light irradiation for 15 min.
2. Wash each channel twice with cell culture medium (4 μL for each time); loading speed for pipette is 6 μL/s. Use pipette tip (connected with vacuum) to remove the exhaust solution.
3. Load 4 μL 30% FBS in cell culture medium into each channel.
4. Put the chip into a Petri dish and drop 0.5 mL sterilized water into the dish to maintain the moisture.
5. Keep the dish in the incubator for 24 hours.
6. Take the dish out of the incubator. Wash each channel twice with 4 μL of cell culture medium; leading speed for pipette is 6 μL/s. Use pipette tip (connected with vacuum) to remove the exhaust solution.
7. Load 4 μL of cell suspension (8×10 cells/mL (˜3200 cells) for cell culture, 2×10⁶ cells/mL (˜8000 cells) for immunostaining) to each channel with pipette loading speed 0.55 μL/s.
8. Put the chip in an incubator (37° C. and 5% CO₂)
9. Keep the chip in an incubator for overnight to ensure good cell attachment.

### Tips:

Bubbles sometimes may be generated in a chip. A drop of culture medium at the inlets helps to decrease the chance to generate the bubbles in a chip.

### Immunostaining:

All the solutions utilized in immunostaining are loaded by pipette with a loading speed of 0.55 μL/s. Similarly, vacuum is employed for removing the exhaust solutions.
1. Put a chip on ice and wash the cell samples by loading 4 μL of 4° C. (cold) PBS.
2. Fix the cell samples ASAP by loading 4 μL of 4% Paraformaidehyde into each channel. incubate chip at RT for 15 min, followed by 4 μl PBS (three times) rinsing.
3. Permeabilize the cells by loading 4 μL of the permeabilization solution (0.3% Triton X-100 in PBS) to each channel. Incubate at RT for 15 min. Subsequently, wash each channel with 4 μL PBS (three times).
4. Prepare blocking solution, consisting of 10% NGS, 0.1% NDBM and 3% Bovine Serum Albumin in PBS. Load 4 μL and maintain at RT for 1 hour.
5. Add 4 μL Zenon fluorophore Labeled antibody solution to each channel and incubate at 4° C. overnight in the darkness. Wash with 4 μL PBS (three times)

### Antibody Solution Preparation:

1. Away from light, add 15 μL (9:1) of Zenon IgG labeling reagent to 1 μg of antibody solution.
2. Wait 5 min.
3. Add 15 μL (9:1) of Zenon IgG blocking solution to the mixture.
4. Wait 5 min.
5. Combine appropriate dilution of antibody mixture with 10% NGS, 0.1% NDBM and 3% Bovine Serum Albumin in PBS.
6. Load in a chip.
6. Fix the cell sample again by loading 4 μL of 4% Paraformaldehyde into each channel. Incubate chip at RT for 15 min, followed by 4 μl PBS (three times) rinsing.
7. Inject 4 μL of the DAPI (10 μg/mL) staining reagent for 5 min and wash the channels with 4 μL of PBS (three times) for each channel.
8. Use Nikon-TE2000 Microscope to detect the fluorescence.

### Tips:

In the case of EGFR staining, since anti-hEGFR is directly conjugated with HiLyte Fluor 750, “antibody solution preparation” is not necessary. Please see “Protocol for antibody labeling with HiLyte Fluor 750”.

### Example C

### Nanotechnologies for Quantitating PI3-Kinase Pathway Biomarkers in Mice and Humans Treated with Kinase Inhibitors

This example takes advantage of a synergy between technological and clinical expertise to develop, optimize and validate microfluidics integrated nanoelectronic sensors as a diagnostic tool for glioblastoma. We set out the following goals for this example:
1) To expand our microfluidics assays for glioblastoma to better characterize mixed populations of cancer cells at the single cell level
2) To begin optimize our ability to perform these microfluidics-based assays on clinical glioblastoma samples to characterize their signaling at the single cell level.
3) To use a series of complementary approaches to develop new serum biomarkers and cell surface markers and to begin to validate some of these new markers.

### 1) Development and Refinement of Chip-Based Microfluidics Integrated Assays for Multiparameter Measurement of Glioblastoma

### 1a) Advances in Chip-Development and Analysis;

Our joint team has recently demonstrated a microfluidic cytometry platform, based on a microfluidic cell array in conjunction with a semi-automated pipette and a fluorescence microscope, for profiling of signaling events involved in the PI3K signaling pathway with single cell resolution. Our initial focus has been on the EGFR/PI3K signaling axis. Drawing on basic mechanistic studies and translating them into the clinic, we have: 1) demonstrated that coexpression of two key proteins that regulate PI3K signaling, a mutant epidermal growth factor receptor (EGFRvIII) and the PTEN tumor suppressor protein are strongly associated with clinical response of glioblastoma patients to EGFR kinase inhibitors and identified mechanisms of resistance associated with PTEN loss (Mellinghoff et al., NEJM 2005); 2) developed strategies to overcome resistance to EGFR kinase inhibitors mediated by PTEN loss (Wang et al., Cancer Res. 2006) and which are being tested in the clinic, and 3) identified additional EGFR mutations that may confer sensitivity to EGFR kinase inhibitors (Lee et al., PLoS Medicine 2006). We have also begun to recognize the challenge of acquired resistance, and to recognize the number of potential routes by which glioblastoma cells may become resistant (Mellinghoff et al., Clinical Cancer Res. 2007).

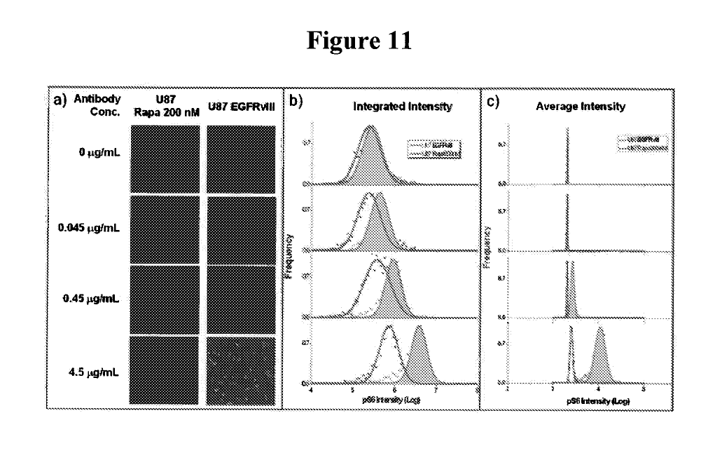

A number of genetically manipulated glioblastoma cells (i.e., U87, U87-PTEN, U87-EGFR, U87 EGFRvIII and U87-EGFRvIII/PTEN) as well as primary cells have been successfully cultured in the devices, and the expression levels of receptors (EGFR and EFGRvIII), protease (PTEN), phosphorylated kinases (p-Akt, pmTOR and p-S6) of the cells can be analyzed quantitatively. In this example, our research effort has been focused on (i) developing robust and reproducible protocols for immunostaining and image acquistion/analysis to achieve optimal dynamic range for our chip-based cytometry measurements, and (ii) creating a user-friendly interface between microfluidic cell array and a robotic pipette, allowing large-scale signaling profiling in a closely-related microenvironment.
(i) Developing robust and reproducible protocols for immunostaining and image acquisition/analysis to achieve optimal dynamic range for our chip-based cytometry measurements. Although our microfluidic cytometry platform can be utilized to generate histograms for signaling pathway profiling, the original conditions/techniques for cellular fixation, permeabilization and antibody treatment, as well as the operation parameters for semi-automated pipette and fluorescence microscope should be further optimized to achieve optimal fidelity and dynamic range for the measurements. As a result, small perturbations in the cell signaling network can be identified by our chip-based cytometry measurements. Based on a systematic approach, we tested different combinations of formaldehyde, methanol, acetone, Triton X-100 to obtain optimal fixation and permeabilization conditions. In addition, phospho-specific antibodies were labeled with Zenon mouse IgG labeling kit and HiLyte Fluorophore reagent to provide multi-color analysis of different signaling events simultaneously within individual cells. Different concentrations of fluorophore-labeled antibodies were examined. Optimized conditions were determined by examining their ability to show maximal separation between the two histograms obtained for the negative and positive control systems. Here, we present an example by which an optimal antibody concentration for p-S6 immunostaining was determined. In this case, the positive control is U87-EGFRvIII glioblastoma cells with amplified p-S6 signal, and the negative control is rapamycin-treated U87 cells, in which p-S6 up-stream signal was blocked. Different concentrations (0, 0.045, 0.45 and 4.5 μg/mL) of Zenon-labeled anti-p-S6 were utilized to treat the two sets of samples. An inverted fluorescent microscope was utilized to acquire fluorescence images (FIG. 11A) which were further analyzed by MetaMorph program (Molecular Device). Notably, the operation/analysis parameters were determined through again the systematic optimization. As shown in FIG. 11B, the antibody concentration of 4.5 μg/mL, gave best separation for positive and negative controls. During imaging analysis, an interesting information, i.e., spreading surface areas of individual cells, were obtained along with their integrated fluorescence intensities. We noticed there was some variation of cell surface areas. This variable factor can be removed by dividing the integrated fluorescence intensities with cell surface areas of individual cells to give dramatically sharpened histogram (FIG. 11C). The resulting average intensity-based histogram gives a better precision and dynamic range for capturing small signals events compared to that obtained from a flow cytometry system. Currently, four standard protocols for immunostaining of EGFR, EFGRvIII, PTEN and p-S6 have been established trough systematic studies.

FIGS. 11A-11C show optimization of antibody concentration for p-S6 staining in a microfluidic cytometry platform. a) Micrographs obtained for the positive (U87-EGFRvIII) and negative (Rapa-treated U87) controls at different anti-p-S6 concentrations of 0, 0.045, 0.45 and 4.5 μg/mL. b) MetaMorph program was utilized to obtain integrated fluorescence intensities of individual cells, and the histograms of p-S6 expression levels were obtained. Apparently, the antibody concentration of 4.5 μg/mL gave best separation for positive and negative controls. c) Average p-S6 expression levels can be obtained by dividing the integrated fluorescence intensities with cell surface areas of individual cells. By removing the factor associated with cell surface areas, the histograms were dramatically sharpened, resulting in better separation of the two histograms.

The following describes collection of immunostaining protocols for PI3K/Akt signaling pathway with some exploration of the associated signaling network. Below, we describe some aspects of this approach to characterize molecularly heterogeneous solid glioblastoma tumor samples using this microfluidic cytometry platform.

### (ii) Creating a User-Friendly Interface Between Microfluidic Cell Array and a Robotic Pipette, Allowing Large-Scale Signaling Profiling in a Closely-Related Microenvironment.

The original microfluidic cytometry platform utilized a semi-automated pipette to handle cell loading, culture media exchange, fixation, permeabilization and antibody staining in sequence. Besides flow injection, most of the operation/process was, in fact, manually controlled. As a result, a significant amount of labor and inevitable operation error constrain further exploration of this semi-automated approach for applications require large-scale studies. In collaboration with our colleagues (Profs. Mike van Dam and Chris Behrenbruch), a user-friendly interface between microfluidic cell array and a robotic pipette (FIG. 12A) has been developed. Our goal is to enable automated operation from initial cell culture/media exchange to immunostaining, so that a large-scale cell culture/assay can be carried out for high-throughput signaling pathway profiling. Such a user-friendly interface composed of two custom-design units, i.e., a chip holder (FIG. 12B) and a pipette tip array (FIG. 12C-12E). The robotic pipette is designated for handling 96, 396 and/or 1536-well plates. To take the advantage of the exiting setting, the chip holder adopted the dimension of a well plate platform, thus it can be directly mounted onto the two plate holders in the robotic system without further modification. Currently, one chip holder can accommodate four microfluidic chips, and there are 40 cell culture/assay chambers in a microfluidic chip. It is important to note that the location of each inlet and outlet holes of cell culture chambers were registered to a specific well location of a 1536 well plate. Therefore, the original program developed for handling 1536 well plates can be employed for programming automated operation of our microfluidic cell culture and assay. The robotic pipette came with eight individually controlled pipette tips, capable of dispensing and withdrawing liquid samples with a 5-nl, precision. In order to integrate our microfluidic chip with the robotic pipette, we reassembled the eight tips into a different orientation, where the eight tips were grouped into four pairs for handling four cell culture chambers in parallel. In each tip pair, a relatively longer tip is in charge of sample dispensing, and the shorter one for sample withdrawing. The elastic property of PDMS-based microfluidic chip allows a good seal between pipette tip and the inlet of the microfluidic chip and small fabrication imperfection. FIG. 12C illustrates how a pair of tips handles sample/solution displacement in a microfluidic cell culture chamber. In contrast to the semi-automated approach which took about 20 minutes to complete routine cell culture medium exchange, the same process can be completed in less than 10 sec using this robotic system (FIGS. 12D and 12E).

FIGS. 12A-12F show a robotic pipette for performing large-scale signaling profiling in an automated fashion. a) A robotic pipette designated for well plate platforms. b) A custom-designed chip holder with a dimension identical to a well plate platform allows convenient interface between the robotic system and microfluidic chips. c) Schematic representation illustrates how a pair of pipette tips is utilized for dispensing and withdrawing sample/solution in a microfluidic cell culture chamber. d) Four pairs of pipettes loaded fixation solutions from reagent reservoirs. e) Automated immunostaining in action. f) A custom-designed replicate for preparation of microfluidic chip with a fixed chip height.

One can implement the new immunostaining protocols for PI3K/Akt signaling pathway profiling using the robotic system according to an embodiment of the current invention. One can automate the image acquisition and processing to provide a complete solution covering sample preparation, image acquisition and data analysis for our microfluidic cytometry technology.

1b) Application of this Technology to Perform Multiparameter Measurement of Key Nodes of the PI3K Signaling Pathway Including in Molecularly Heterogeneous Samples. This Device has Obvious Implications for Glioblastoma, but is also likely to be Quite Important for Analysis of other Cancer Types. Quantitative Single Cell Detection of Upstream Signaling Proteins—EGFR, EGFRvIII and PTEN in Glioblastoma Cells:

We have made significant advances in our ability to quantify these upstream markers of PI3K signaling in glioblastoma cells, at the single cell level. Here, we now show how quantitative analysis can be performed simultaneously on multiple signaling proteins of the PI3K pathway in a highly quantitative fashion, at the level of single cell resolution. Further, we show that such data can be analyzed in a similar fashion to flow cytometry data, to facilitate direct comparisons, and to allow for analysis of signaling profiles in complex heterogeneous mixtures, including before and after molecularly targeted treatments.

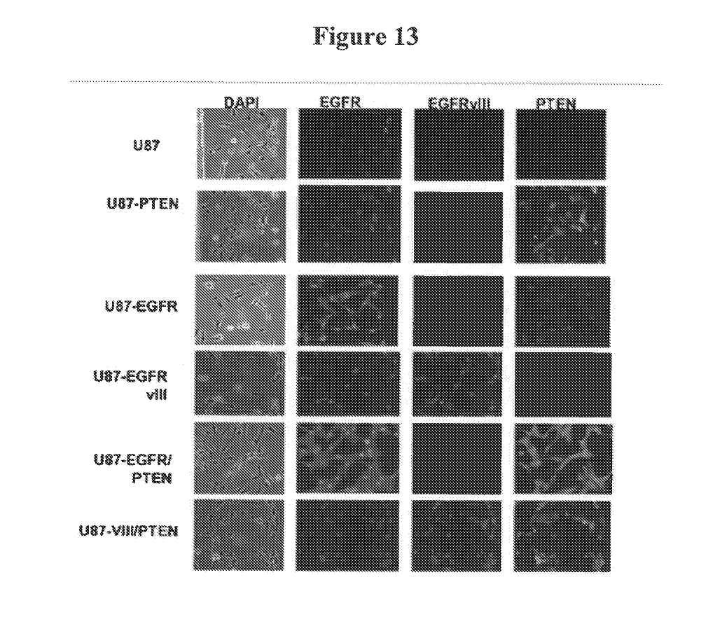

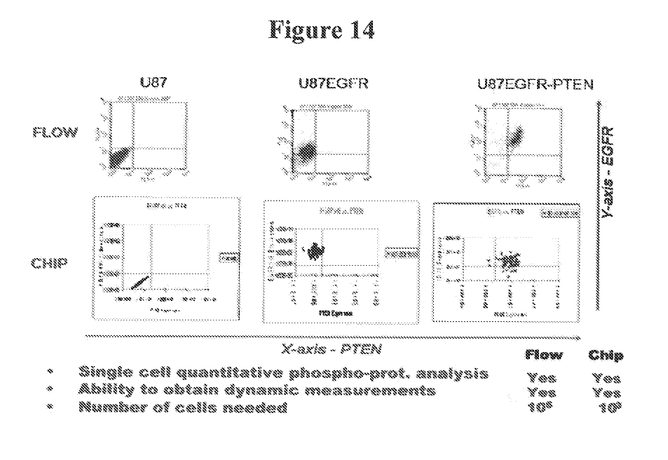

FIG. 13 shows the immunofluorescent staining of the six isogenic glioblastoma cell lines varying in EGFR, EGFRvIII and PTEN, performed on chip. FIG. 14 shows the quantitative analysis, as compared to flow cytometry performed on the same samples. As demonstrated, this on-chip based approach can characterize these key upstream markers in single cells like flow cytometry, however with far fewer cells required.

FIG. 13 shows on chip multiparameter measurement of upstream markers of the PI3K pathway in glioblastoma EGFR, EGFRvIII, and PTEN.

FIG. 14 shows EGFRvIII, EGFR, PTEN detection—comparison of FLOW vs. CHIP—same cell suspensions were run in parallel on chip and by flow. Quantitative detection on chip matches that by flow, but requires far fewer cells.

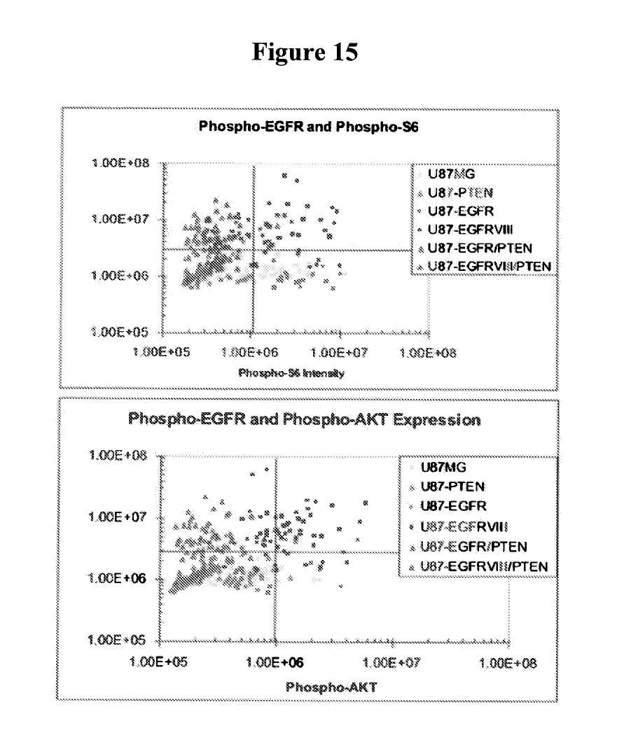

FIG. 15 shows quantitative measurement of PI3K signaling (p-EGFR, p-akt, p-S6) in single cells from molecularly complex heterogeneous glioblastoma samples. The six isogenic glioblastoma cell lines were cultured together as a complex mixture and then analyzed on chip for expression of EGFR, ak, and S6 phosphorylatin. The data are plotted in a “flow cytometry” format. As seen in the panels, akt and S6 phosphorylation are relatively elevated in all of the PTEN deficient cells, while constitutive EGFR phosphorylation is greatly enhanced by the oncogenic mutant EGFRvIII, relative to the wild type receptor. Thus, it is clear that the EGFRvIII expressing, PTEN deficient tumor cells (blue dots), one-sixth of this mixture, are the cells with the most PI3K signaling activation, consistent with our prior findings (Mellinghoff et al., NEJM 2005), and that this can be measured on a single cell basis in molecularly heterogeneous tumor samples.

### Quantitative Single Cell Detection of Downstream Signaling Effectors of the PI3K Pathway—Akt and S6 in Glioblastoma Cells:

Expression of the constitutively activated EGFR, EGFRvIII, and loss of the PTEN tumor suppressor have known implications for constitutive activation of PI3K signaling (Mellinghoff et al. NEJM, 2005). Thus, it is expected that these signaling proteins should be activated in the same cells in which EGFRvIII is expressed and/or PTEN is lost. Thus, we farther analyzed these downstream markers on chip in the same cells in which EGFRvIII is overexpressed and PTEN is lost. As shown below in FIG. 15, in which the six isogenic glioblastoma cell lines are mixed (U87, U87-PTEN, U87-EGFR, U87-EGFR-PTEN, U87-EGFRvIII and U87-EGFRvIII-PTEN) and analyzed on chip with the data presented in a “flow cytometry” format, it is clear that constitutive activation of the PI3K signaling pathway, as measured by akt and S6 phosphorylation is observed in the EGFRvIII expressing, PTEN deficient tumor cells. These results demonstrate the feasibility of identifying unique and clinically relevant molecular signatures in complex heterogeneous tumors.

### Measurement of PI3K Signaling in Molecularly Heterogeneous Glioblastoma Samples:

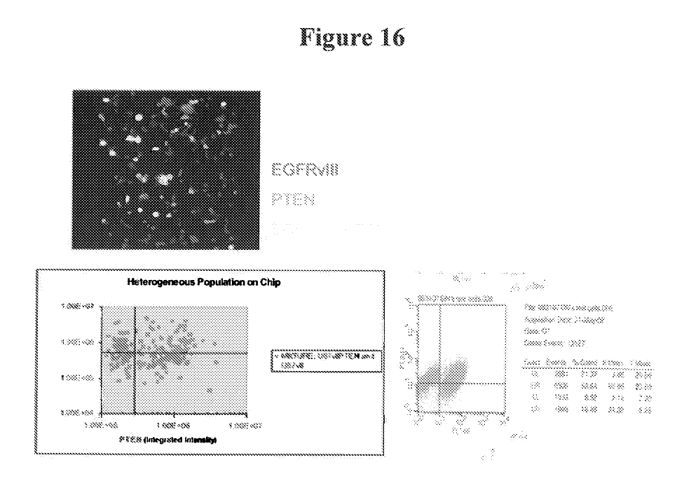

It has long been recognized that glioblastoma is one of the most molecularly heterogeneous of all tumors, hence the name glioblastoma multiform. This heterogeneity refers to the striking phenotypic and molecular variability of individual cells within a single tumor. Thus, one of the aims of this example is to develop tools to characterize the signaling pathways within these molecularly diverse individual cells. FIGS. 13-16 above demonstrate the ability of the microfluidics-integrated chips we have developed to measure the key proteins of the PI3K signaling pathway in a mixture of cells. The next demonstrations below, further show how this on-chip approach can sort out individual populations within a glioblastoma and characterize their signaling including within “rare” cells within a tumor. In FIG. 16 below, we mixed two isogenic glioblastoma cell types (U87-PTEN and U87-EGFRvIII) in a 1:1 ratio and examined whether these populations could be distinguished on chip, as well as by flow cytometry. As seen below, two distinct populations of cells can be identified. (See FIG. 16.)

FIG. 16 shows characterization of a molecularly heterogenous population on a chip. U87-PTEN and Ub7-EGFRvIII cells were mixed in a 1:1 ration and examined on-chip (left panel) and by flow cytometry (right panel). As demonstrated, a molecularly heterogenous sample can be detected on chip.

To determine whether we could identify a “rare” population of cells on chip based on expression of key signaling markers, we mixed U87 (95%) and U87-PTEN-EGFR (5%) and examined the populations on chip. As shown in FIG. 16 below, we were able to detect the rare population of cells, measured to be 7.4% (+/−2%) of the population. This was also consistent with the assessment by flow cytometry, but it required a far smaller number of cells (tens to hundreds, not thousands). This raises the possibility that we will be able to identify rare subpopulations of cells, such as brain cancer stern cells, based upon key signaling or cell surface markers.

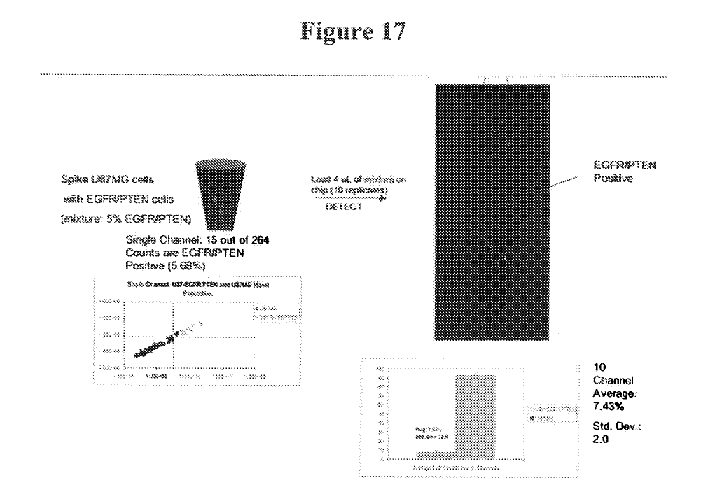

FIG. 17 shows On Chip Detection of “rare cell” within a population. A mixture of U87 cells (95%) and U87-EFGF-PTEN cells (5%) were analyzed on-chip. As shown, we could detect the rare population of cells, measured to be 7.4% (+/−2%).

Multiparametcr Measurement of Key Signaling Pathways from a Solid Clinical Tumor Sample.

Most of the advances in the field of single cell analysis have come from non-solid tumors such as leukemias and lymphomas, which can be easily dissociated into single cell suspensions for characterization. One of the chief challenges for solid cancers is to adapt these technology-based approaches to characterize signaling in solid tumor samples. We have begun to make significant progress in this area. As an important intermediate step, we have begun working with human serially passaged glioblastoma intracranial xenografts developed by C. David James (at UCSF) and him Sarkaria (at Mayo clinic) (Sarkaria et al., Mol. Cancer Ther. 2006). These models consist of serially passed human tumors that retain the key characteristics of glioblastoma, such as EGFRvIII expression, which are lost in normal culture and xenograft systems. Using this model enables us to best optimize our approaches using a renewable clinical resources which is for all intents and purposes a solid, molecularly heterogeneous clinical tumor sample, much like the ones coming straight from the operating room. Below, we demonstrate considerable progress in this area. In parallel with this, we are optimizing our preparation techniques to go from a solid tumor sample from the operating room to the chip, with no need for tissue culture (which significantly changes the molecular composition of the tumor). We have made significant progress in this area as well. We now have developed an approach to enable us to go directly from a tumor sample to a chip. The chief obstacle has been the difficulty of getting the cells to “lie down” on the solid chip. We have now developed some approaches to facilitate this, which do not change the signaling characteristics of the tumor cells.

### Multiparameter Measurement of EGFRvIII and PI3K Signaling in a Solid GBM Tumor Sample.

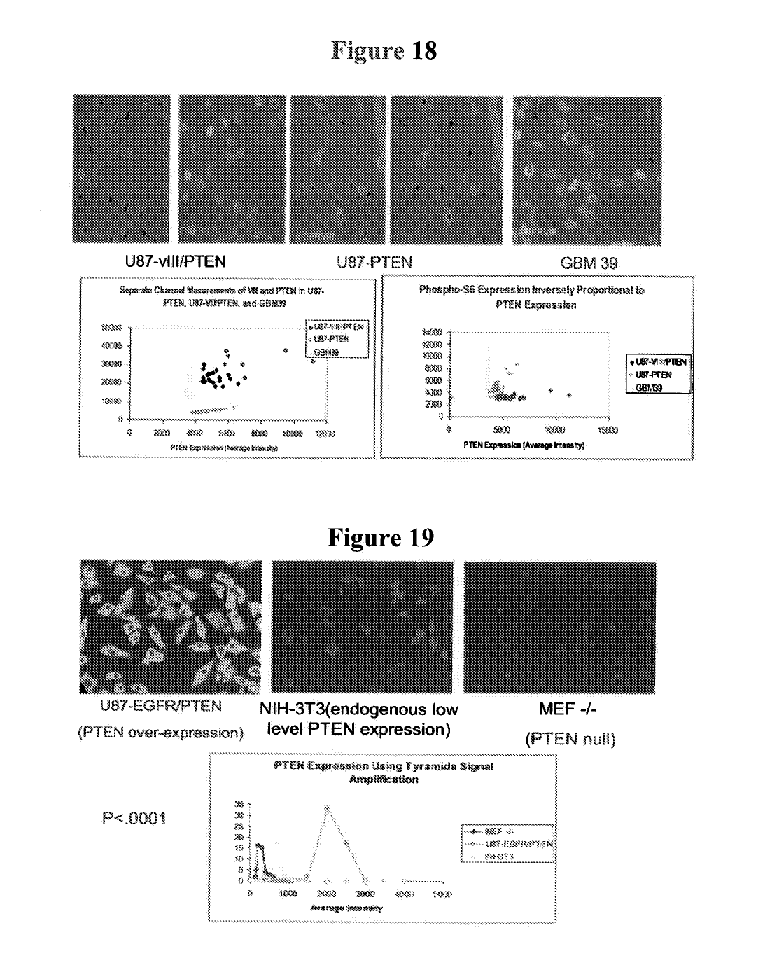

To begin to analyze EGFRvIII and PI3K signaling in a solid clinical sample, we used GBM 39 from the James/Sarkaria model system. After obtaining a solid piece of tumor, we dissociated with collagenase and mechanical means and optimized a series of approaches for moving onto chip followed by analysis of PI3K signaling. As shown, below, we present, to the best of our knowledge, the first demonstration of single cell quantification of key nodes of the PI3K signaling pathway in a solid GBM sample. The data demonstrate a tumor with with EGFRvIII expression and significant PI3K pathway activation (see FIG. 18).

FIG. 18 shows detection of PI3K signaling in a solid clinical glioblastoma sample. We analyzed expression of EGFRvIII, PTEN, and S6 phosphorylation (as a readout of PI3K signaling) from a clinical solid glioblastoma sample GBM 39 derived from the in vivo serially passed GBM model (Sarkaria. . . James, Mol. Cancer Ther. 2006). U87-EGFRvIII-PTEN and U87-PTEN cells were also analyzed at the same time to provide a basis for comparison. Note expression of EGFRvIII (left panel) and activation of PI3K signaling (right panel) in GBM 39. This is, to the best of our knowledge, the first single cell quantitative measurement of EGFRvIII and PI3K signaling in a solid GBM sample.

Addressing the challenge of measuring endogenous levels of key signaling proteins—PTEN measurement. We highlight the challenge that some of our antibodies do not provide an ideal signal to noise ratio. When combined with the relatively low level of expression of certain endogenous proteins such as PTEN, this raises a challenge. We have adapted a tyramide signal amplification strategy to increase our signal to noise ratio for PTEN detection, which enables us to detect endogenous levels of PTEN while allowing us to continue to multiplex the measurement reactions. In FIG. 19 below, note that this approach has enabled as to detect and quantify PTEN expression not only in the context of PTEN overexpression, but also for endogenous PTEN expression, with no detection of PTEN expression in PTEN null mouse embryonic fibroblasts (MEFS). These data demonstrate that we are likely to have the sensitivity and specificity to detect low-level endogenous proteins for measurement. During the next reporting period, we aim to further refine this approach. (See FIG. 19).

FIG. 19 shows detection of low levels of endogenous PTEN. Using tyramide signal amplification, we are able to measure: a) PTEN in an overexpression system, U87-EGFR-PTEN, left panel; endogenous low level PTEN in NIH 3T3 cells, middle panel; and lack of PTEN in PTEN null MEFs, right panel. Below, not quantification of PTEN expression.

2. Development of Technologies to further Assess Pathways of Cancer at a Single Cell Level.

We have begun using the DEAL, DNA-encoded antibody libraries, coupled with development of new cell surface markers to develop new strategies for isolating and enriching single cell populations.

2a) DEAL technology we have validated the ability of DEAL-based approaches for sorting cells based on EGFR expression. We are currently working on incorporating new cell surface markers into this process. We have developed a panel of novel cell surface markers, validated by flow cytometry and western blotting, which we plan to incorporate into this DEAL strategy.

2b) Detection of novel cell surface proteins and secreted proteins. We have leveraged global gene expression data from clinical samples (63 GBMs) relative to 20 normal brain samples and identified potential cell surface markers that are likely to be highly overexpressed in GBM cells. We have then gone on to analyze the expression in a large cohort of GBM and grade III gliomas patients, to confirm the the mRNA level overexpression of these markers in GBM (and their significant overrexpression in GBM relative to grade III tumors). We have then gone on to examine their protein level expression in GBM cell lines, then in a series of matched glioblastoma/normal pairs from patients on whom autopsies were done. We have then begun to analyze in vitro, the potential relationship between these markers and the EGFR/PI3K signaling axis. To date, we have identified 10 potential cell surface markers including: PSCI.R1, CXCR4, and PTRPZ1 (also identified as a secreted protein—see approach 2 below).

2c) Identification and Validation of Novel Secreted Proteins:

Through global MPSS data analysis on differential gene expression of brain tissue samples from glioblastoma patients versus non-tumor patients, approximately 5000 genes were found to be differentially expressed in glioblastoma brain tissue samples. Thirty eight genes whose expression is relatively enriched in brain were selected for targeted screening of serum samples from glioblastma patients as blood diagnostic hiomarker candidates for glioblastoma. In the mass spectrometry based targeted proteomics approach that is being developed at ISB, proteins from serum samples are first digested to peptides and all peptides are labeled at the N-terminus with stable isotope labeled reagents called iTRAQ (from Applied Biosystems). Peptides from up to four samples can he differentially labeled with iTRAQ reagents for measurement of relative abundance of 100s of proteins across the four different samples. In order to overcome the difficulty of quantifying low-abundance proteins in serum by mass spectrometry due to the sample complexity, two techniques have been utilized: (1) several predominant serum proteins can be selectively removed by immunoaffinity-based depletion methods; (2) the detection of low abundance peptides by mass spectrometer is significantly enhanced by increasing the total amount of the selected peptide in the ITRAQ labeled mixture—84 peptides from 38 candidate blood protein markers were synthesized at low cost and mixed with one of the iTRAQ reagents while two peptide samples from normal and tumor patients were labeled with the other two iTRAQ reagents. The data analysis of this experiment is currently under way. As a follow up of this, we have isolated a set of 25 matched tumor normal brain paired samples and have begun screening for differentially expressed proteins by Mass Spec.

2d) Integration of these New Technologies into Molecularly Guided Clinical Trials:

We are now in the process of a clinical trial of patients treated with the VEGF inhibitor Avastin (in combination with CPT-11), we can now demonstrate that Avastin nearly quadruples time to tumor progression from a median of 56 days (95% CI=49-97) to 209 days (95% CI=122-272) and nearly doubles overall survival from a median of 184 days (95% CI=171-225) to 340 days (95% CI=251-424) for patients with recurrent malignant gliomas. The obvious clinical efficacy of avastin will most certainly change the standard of care for malignant glioma patients. In addition, the fact that we have a relatively large number of clear clinical responders (as well as non-responders), with frozen tissue (as well as paraffin tissue) obtained at the time of diagnosis, as well as frozen tissue in a subset of patients who had a clinical response, but then failed treatment, and serum obtained before treatment, during clinical response and at the time of treatment failure provides a remarkable coordinated molecular/clinical resource for identifying the molecular determinants of response to this drug and to identify effective targeted combinations. We have a relatively large number of clear clinical responders (as well as non-responders), with frozen tissue (as well as paraffin tissue) obtained at the time of diagnosis, as well as frozen tissue in a subset of patients who had a clinical response, but then failed treatment, and serum obtained before treatment, during clinical response and at the time of treatment failure. We will address the following questions: 1) does increased vascularity predispose towards clinical response to avastin (this will be done in collaboration with Dr. Luisa Iruela-Arispe, a noted expert in angiogenesis at UCLA); 2) are the downstream signaling pathways regulated by VEGF reactivated during response and by what mechanism. Are tumors which are driven by key pathways that promote VEGF expression (i.e. EGFR/PI3K signaling) more sensitive to avastin? In addition to testing lese specific hypotheses, we will apply global screening strategies. These include array CGH to detect chromosomal regions of interest associated with sensitivity and resistance (will be done at MSKCC by Dr. Mellinghoff) and integrated with global gene expression data (done both at UCLA and at MSKCC), in addition, through our NanoSystems Biology Cancer Center, we have begun collaboration with Dr. Lee Hood to use surface Plasmon resonance to facilitate the quantitative detection of thousands of proteins in a serum samples. This will enable us to identify protein signatures associated with sensitivity and acquired resistance.

3. Systems Biology Approaches Towards Identification of New GBM Targets:

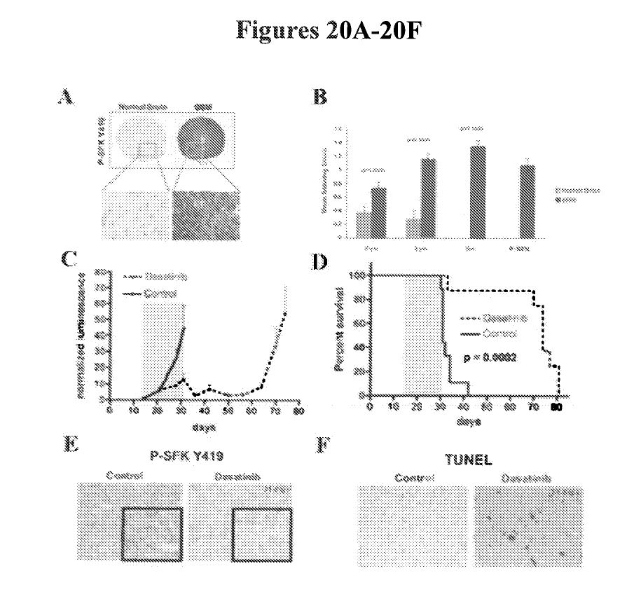

3a) Targeting the Src Family Kinases: Targeting EGFR/PI3K signaling provides a proof-of-principle for the potential efficacy of molecularly targeted therapy for glioblastoma. Identifying new drug targets is a critical next step. We have developed and applied systems biology approaches to identify novel molecular targets in glioblastoma. Integrating global gene expression data from GBM samples with molecular interaction databases to identify potential targetable pathways, we have identified the Src family kinases (SFKS) Fyn, Lyn and Src as molecular targets in glioblastorna. We leveraged global transcriptome data from glioblastoma clinical samples to identify and validate the Src family kinases (SFKs) Src, Fyn and Lyn as key molecular targets We demonstrated that these kinases are overexpressed and persistently phosphorylated in up to 50% of glioblastoma patients, demonstrated using both siRNA, small molecule inhibitor and over-expression studies that the SFKs are necessary and sufficient to promote glioblastoma invasion and we have demonstrated that the small molecule inhibitor dasatinib (a compound safely given to patients with imatinib resistant CML) inhibits glioblastoma invasion, promotes tumor regression and apoptosis and greatly enhances survival in a mouse model in vivo. (See FIG. 20).

In FIG. 20, the SFKs are dasatinib sensitive molecular targets in GBM. A, B) Elevate SFK phosphorylation is seen in up to 50% of GBMs. C, D) A short window of dasatinib treatment (gray shadow) causes tumor regression (as measured by optical luminescence imaging, collaboration with Dr. David James) and significantly enhances survival in vivo in an intracranial human serially passaged GBM xenograft model. E, F) Dasatinib inhibits SFK phosphorylation and promotes apoptosis in vivo.

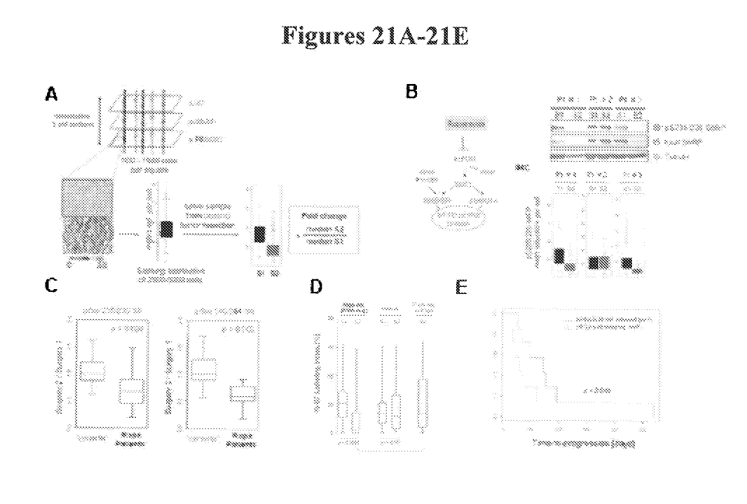

3b) Studying the mechanism of mTOR mediated inhibition in vivo: We conducted clinical trail of the mTOR inhibitor rapamycin in patients with relapsed, PTEN-deficient glioblastomas and identified key determinants of sensitivity and resistance (Cloughesy et al. 2007, PLoS Medicine, in press). We showed that: 1) the mTOR inhibitor rapamycin is present in potentially therapeutic levels in tumor tissue in vivo; that 2) rapamycin significantly inhibits mTOR signaling in all patients although the extent of inhibition is variable (from 10%-80% pathway inhibition) and that 3) the extent of pathway inhibition is critical. mTOR pathway inhibition of greater than 50% resulted in significantly inhibited proliferation; lower levels of mTOR inhibition did not translate into biological or clinical response, We further showed that that was not due to cell intrinsic resistance, but more likely was associated with failure of the drug to fully access its target in vivo. Finally, we studied the relationship between growth inhibition and sustained clinical response and found evidence for an Akt mediated feedback loop nearly half the patients. This Akt-mediated feedback loop was significantly associated with acquired clinical resistance. These findings have important implications. First, they indicate that in future clinical trials, it will be critical to develop approaches to document sufficient target inhibition in glioblastoma patients in order to interpret clinical activity. Second, these findings further point to the value of combined EGFR/mTOR blockade (as also suggested by item I.2. above). Third, the data on the Akt-mediated feedback loop suggests the importance of combined PI3K/mTOR inhibition to better inhibit this pathway and to suppress the upstream feedback loop. These insights will soon be put into practice in a series of clinical trials (in addition to the EGFR/mTOR Inhibitor trials). (See FIG. 21).

FIG. 21 shows the development of an approach to measure effect of mTOR inhibition in glioblastoma patients and demonstration of a resistance promoting feedback loop. A) We developed an image analysis approach to compare biomarkers in patients pre- and post treatment with rapamycin. B) This immunohistochemical method is validated by traditional biochemistry. C) Rapamycin significantly inhibits mTOR in glioblastoma patients in vivo and D) inhibits tumor cell proliferation, as measured by Ki67. E) Rapamycin treatment activates a feedback loop through Akt in a subset of patients (measured by the PRAS 40 biomarker) and this activation leads to significantly shorter time to progression.

3c) Measuring EGFRvIII in vivo. We developed a novel nucleic-acid based approach for reliable detection of the mutant EGFRvIII (Yoshimoto et al., Clinical Canceer Res, 2007, in press). We have previously shown that EGFRvIII (in the context of intact PTEN) sensitizes gliohlastomas to EGER inhibitors (Mellinghoff et al., NEJM 2005). EGFRvIII also presents a unique antigenic target for anti-EGFRvIII-directed vaccines. Thus, detection in clinical samples may be warranted. However, frozen tissue is not routinely available, particularly for patients treated in the community. Thus, detection of EGFRvIII in formalin-fixed paraffin embedded (FFPE) clinical samples is a major challenge. We developed a real-time RT-PCR assay for EGFRvIII from routinely processed formalin fixed, paraffin-embedded glioblastoma biopsy samples that is 92% sensitive and 98% specific. This assay will be readily available to pathology laboratories everywhere to facilitate widespread testing of EGFRvIII mutation in glioblastoma patients.

### B) Further Embodiments

B.1: Expanded microfluidcs assays. One can continue to expand our analysis on-chip of the key signaling pathways in glioblastoma according to embodiments of the current invention. One can further increase the number of signaling proteins being currently measured, assess our ability to study the effects of signal transduction pathway inhibitors on-chip, including in mixed populations of GBM cells, and integrate these chips with the cell surface sorting approaches described above. We can also begin working more with fresh tumor samples from patients to optimize approaches for quantifying the molecular heterogeneity and for optimizing on-chip culture of samples.

B2: Optimization of detection of key signaling pathways in routinely processed clinical samples (and in retrospectively frozen clinical samples). One can apply this to solid clinical tumor samples to optimize multiparameter measurement of solid clinical samples. We can also aim to develop ways of examining previously frozen samples, especially those from patients on molecularly based clinical trials, as this can allow us to make correlations between signaling states and response to targeted therapies in trials we have already performed.

B3. Assessment of whether molecular heterogeneity is global or local. The extert of molecular heterogeneity within glioblasioma has been recognized, but not rigorously studied. In fact, it is unclear whether local heterogeneity within a tumor is representative of the entire tumor, or whether spatially dispersed biopsies are required for a complete molecular characterization of a tumor. The answer to this question has obvious importance for designing rational combination therapies for patients to suppress resistance. The development of these tools can facilitate our asking this question.

B4. Pathways of a cancer at a single cell level: We can expand our DEAL approaches using antibodies to the cell surface proteins described above. We can further integrate these DEAL approaches onto the microfluidics based chips.

B5. Development of new serum markers: The UCLA, ISB and CIT groups may integrate their potential serum marker lists. UCLA and ISB can both obtain serum samples, as well as tumor samples, and we can perform larger scale analysis of potential serum proteins using the ISB-based approaches.

B6. Further identification and validation of cell surface markers: We can continue to analyze the expression of the list of potential cell surface markers, begin to determine whether sorting based on these markers (DEAL and flow based) identifies populations of GBM cells with different phenotypic and biochemical properties (and different patterns of sensitivity and resistance to targeted agents).

### Example C

### The Current Model of Pathology

The current model of pathology diagnosis for cancer is based on the microscopic resemblance of cancer cells to their presumed cell of origin or its developmental precursor. Based on tissue morphological appearance, as well as the presence or absence of a few protein markers, the pathologist concludes a broad pathological diagnosis conveying tumor type and grade. Typically, the patient is treated with relatively toxic, non-specific therapies such as DNA damaging agents and radiation. While this classification and affiliated grading system has proven to be useful for predicting the overall survival for groups of patients and for communicating broad information about the disease category²⁴⁻²⁹, relatively limited insight is gained about the underlying molecular pathway lesions³⁰. Furthemore, clinically relevant subsets that may differ significantly in their time course and responses to therapy cannot be monitored with the current classification system. Traditional pathological examination, considered to be the “gold standard” of cancer diagnostics, may not be well-suited towards molecularly targeted approaches because lineage type distinctions based on morphology do not reveal information about the underlying molecular networks.

Cancer is a disease of molecular heterogeneity³¹⁻³⁵. The past decade has clearly demonstrated that the underlying molecular lesions in tumors of the same histological type are quite different, More importantly, patients with cancers of the same histological type may respond quite differently to therapy depending on the molecular composition of their tumor. There is emerging evidence for considerable molecular heterogeneity within individual tumors, for example a small population of tumor repopulating stem cells, which may be the key drivers of cancer recurrence. There is considerable need to develop robust quantitative tools for multi-parameter measurement of signaling networks within the individual cells of a tumor. It is now clear that any type of cancer stratifies into a set of diseases, each with its own molecular signature. Traditional pathological examination cannot distinguish these relevant tumor subsets, because they are usually microscopically identical.

The advent of targeted therapy has led to the development of a variety of target-specific drugs (e,g., kinase inhibitors³⁶), and have demonstrated profound outcomes in several types of cancer^{6,37-42}. The implementation of targeted therapy necessitates a new molecular diagnostic approach for identification and quantification of disease targets associated with the exact signaling pathway responsible for the malignant transformation of cancers. Conventional technologies for molecular analysis (e.g.. Western blot) that require a significant amount of tissue are constrained for dynamic and single-cell characterization. One of the critical challenges for molecular diagnosis is multi-parameter measurement of the signaling pathways within the molecularly diverse cells with single cell precision.

### A System Approach to Cancer Pathology

### Cancer is a Complex Disease

No two individual tumors are identical. Analyzing individual genes or proteins in isolation is unlikely to yield important breakthroughs for the diagnosis and treatment of cancer. In contrast, a systems biology approach⁴³⁻⁴⁶ to cancer aims to define the protein and gene “modules” and networks that are responsible for the emergent properties of cancer (i.e. their proliferative capacity, their invasive capacity, their resistance to therapeutic inhibitors). By capturing information about relationships between key elements of the system, commonalities between highly individual cancers can be understood and targeted.

### Integration of Complex Inputs

The systems approach involves taking as many molecular signatures of gene and protein expression as possible as the input, as well as phenomenological information, and integrating them into a network using graphical models. As more inputs are integrated, the structure of the networks is refined enabling generation of hypotheses about how the system works (or in the case of cancer, how it has gone awry). These hypotheses can then be dynamically tested by performing a series of systematic perturbations and measuring the effects on the network (and on phenotypic properties such as growth, invasion, response to therapy, etc). This allows for modification of the hypotheses, followed by further testing and refinement. A more complete and molecular “snapshot” of the system is possible and this high-content knowledge can translate into new diagnostic and therapeutic tools thereby redefining the pathological diagnosis of cancer.

### New Technologies are Required for a Systems Approach to Cancer

With the current suite of available technologies, a true systems level evaluation of cancer is impossible. However, our group has been at the cutting edge of an emerging set of molecular and nanotechnologies that is being integrated into a systems biology laboratory. The development and validation of the Microfluidic Image Cytometry (MIC) technology described here will facilitate a new type of pathologic examination of cancer cells and tissues; chiefly, one that incorporates a systems level approach to diagnosis.

### The PI3K/Akt Pathway is an Important Target in Cancer

PI3K⁴⁷ is a lipid kinase that promotes diverse biological functions including cellular proliferation, survival and motility⁴⁸⁻⁵¹. The PI3K signaling pathway regulates various cellular procosses, such as proliferation, growth, apoptosis and cytoskeletal rearrangement. The PI3K signaling pathway is frequently deregulated in a majority of human cancer types^{52,53}, often in combination with the ER pathway⁵⁴⁻⁵⁶. The PI3K-AKT-mTOR pathway⁵⁷⁻⁶¹ (and RAS/ERK pathway^{62 63}) can become deregulated on the basis of oncogene activation and tumor suppressor gene losses that are commonly seen in glioblastoma⁶⁴⁻⁶⁶. A significant portion of cancer cell types contain alterations of the PTEN tumor suppressor gene⁷⁻⁹, a negative regulator of PI3K signaling, which results in constitutive activation of the PI3K pathway⁶⁷, Upstream of PI3K, the epidermal growth factor receptor (EGFR) is commonly overexpressed^{39,64,68-71}, frequently in association with its constitutively activated EGFRvIII variant (and other variants)⁷¹⁻⁷⁵, often leading to deregulated PI3K and RAS/ERK signaling⁶². The PI3K and RAS/ERK pathways connect richly to other signaling cascades, thereby integrating signals associated with other cell surface events, stress activation pathways and extracellular matrix proteins. Clearly, the PI3K and ERK signaling pathways, and associated signaling molecules, are important therapeutic targets.

### The Signaling Profile by Flow Cytometry

Flow cytometry⁷⁶⁻⁷⁹ can track and analyze signaling events in individual cancer cells. Flow cytometry's unique capability to quantify multiple properties of individual cells can provide information for each cell in a heterogeneous mixture. Cells are usually fixed and permeabilized to allow access by various reagents. Cells with signaling molecules of interest are detected using a fluorophore-conjugated antibody that is specific for recognition of the protein. The single-cell resolution and multi-parameter nature of flow cytometry data can produce signatures to distinguish between cancer cells and non-tumor cells. Because cells have to be dissociated for detection, flow cytometry has primarily been used to study hematological caricers^{80,81}. Adherent tumor cells from solid tumors must be dissociated or detached prior to the flow cytometry measurement. In this way, the signaling pathways of the cells are potentially perturbed and false readouts are possible. The ultimate limitation to flow cytometry is the large number requirement of cell sample (on the order of 10⁶) making it impossible to interface with needle biopsy and other minimally invasive biopsy sampling techniques.

### Integrated Microfluidic Systems and the Development of the “Lab on a Chip”

Microfluidic systems are ideal platforms for handling tumor samples for many intrinsic advantages including sample economy, precise fluidic delivery, scalability and digital controllability⁸²⁻⁸⁷. Cell culture and cell assays can he performed in microfluidic devices^{72,88-97}. Multiple culture chambers can be incorporated in a single chip, allowing multi-parameter analysis with high fidelity, Unlike a microplate-based cell culture system, the microfluidic chip offers a 3-D culture environment that better mimics an in-vivo microenvironment. Controlled unidirectional fluid flow improves the fidelity of biological assays. Some researchers in this field include Dr. Steven Quake at Stanford, Dr. Luke Lee at UC Berkeley, and Dr. David Beebe at the University of Wisconsin, Madison. Dr. Quake implemented a microfluidic bioreactor for long-term culture⁷² of mammalian cells and monitoring of extremely small populations of bacteria at the single-cell level⁹⁸. A microfluidic cell culture device for mammalian cells⁹⁸⁻¹⁰² and yeast has been developed at UCB and is now a commercial product. Dr. Beebe has achieved some interesting cellular microenvironments on a chip by exploiting diffusion-limited mixing and other phenomena unique to the microscale^{96,103,104}. The research field is well-developed and characterized now reaching a stage where “Lab on a Chip” devices can reliably be exploited to accelerate much-needed progress in other stagnant or monumentally complex research fields, but only if driven with economic and solutions-based initiatives.

### An Innovative Technoloy Platform: Microfluidic Image Cytometry

MIC technology according to some embodiments of the current invnetion can perform quantitative and multiparameter immunocytochemistry (ICC) with superb precision and data fidelity. Our objective for some applications is to detect a collection of biomarkers associated with a cancer signaling network responsible for the malignant transformation of cancer. The resulting molecular signature can aide in better cancer diagnosis and the implementation of targeted therapy. The MIC platform integrates three functional modules, including (i) an economic PDMS-based microfluidic cell array chip for supporting the culture of primary cancer cells; (ii) a semi-automated pipette or a robotic pipette for performing cell seeding/culture and ICC, and (iii) an associated data acquisition (fluorescence microscope) system plus sophisticated software for scalable high-content analysis that is well-suited for clinical cancer applications. A series of protocols has been established for measuring the expression/phosphorylation levels of six biomarkers simultaneously (EGFR, EGFRvIII, PTEN, pAKT, pS6 and pERK) in both the PI3K-Akt-mTOR and RAS-RAF-MAPK signaling pathways. Multi-parameter signaling profiles of heterogeneous cell populations from primary tumor tissue are now possible. We also demonstrated the ability of MIC technology to track dynamic changes of the signaling events in the cancer cells upon the exposure to exogenous stimuli (e.g., drugs and growth factors). We are further applying MIC technology to detect signaling events beyond a single pathway, for example, the cross-talk between the PI3K-AKT-mTOR and RAS-RAF-MAPK pathways and feedback interactions involved m-TOR, IRS1 and pAKT. Some aspects of the MIC technology is summarized as the follows:

1. Enabling in-vitro molecular diagnostics. The MIC technology can harness the advantages of microfluidics (i.e., sample economy, speed, scalability, automation and reproducibility) into a solutions driven package to facilitate a clinical pathologist's characterization of an individual's cancer at a molecular and conclusive level. We are pioneering the concept of single-cell signaling profile in a patient's cancer cells.

2. Significant sample economy. Commonly used techniques for protein quantification in biology and clinical laboratories such as Western blot and flow cytometry require at least 10⁴ cells for a single measurement. MIC technology can quantify four signaling molecules using only 200 cells in less than 1.0 μL of media. Consumption of antibodies is dramatically reduced in MIC technology in contrast to Western blot and flow cytometry. The sample economy in MIC technology allows a perfect conjunction for analyzing precious pathology and cytology samples which have limited quantity and are unable to be analyzed by Western blot, flow cytometry, ICC and immunohistochemisny. The single-cell resolution and multi-parameter nature of the MIC data clearly distinguishes the signaling signatures of cancer cells from non-cancer cells, thereby tackling the cellular and tumor heterogeneity issues and far surpasses the performance of competing technologies.

Cost-efficient operation and easy adoption. The cost of carrying out a semi-automated MIC measurement can be fairly low. Our manufacturing strategy can lead to low cost of a MIC chip that is extremely competitive in the market for the high-value added. The intermediate automation liquid handling solution is attainable at a relatively low cost investment for the greater through-put achieved, The florescence cytometry images are acquired on most fluorescent microscopes equipped with reasonable CCD cameras and standard filter sets.

4. Add-on cell sorting capacity. The proprietary chip manufacture strategy can allow convenient incorporation of functional surfaces onto the substrates of the MIC chips. For example, a streptavidin coating or DNA array can be introduced onto the MIC chips allowing immobilization of biotinylated and DNA-tethered antibodies specific to cancer cell surface markers to achieve an on-chip cell capturing function. A sophisticated signaling profile on subsets of cancer cells is possible by first performing on-chip cell sorting followed by the MIC signaling profile on a collection of biomarkers.

Large-scale integration. MIC technology can achieve high through-put cell culture and assay. Various mammalian cell types including, but not limited to cancer cell lines and embryonic stem cells, can be cultured in chambers permitting multiple isolated experiments or in parallel or in duplicate by integrating and automating cell-handling and preparation steps. MIC technology can generate reproducible high content data and quantitative analysis, which can be utilized for applications in large-scale drug screening.

### Example Studies and Results

Microfluidic Imaging Cytometry (MIC) technology integrates a microfluidic cell array^{88,89} with a pipetting robot (for performing sample preparation and immunocytochemistry) and an automated fluorescence microscope (for image acquisition and analysis). MIC technology has been used to perform quantitative and reproducible immunocytochemistry (ICC) for multiple protein detection in a signaling network, using only a small amount of biological samples. A number of glioblastoma cell lines, as well as genetically modified and primary glioblastoma cells were used to validate the MIC technology. The expression/phosphorylation levels of six signaling proteins associated with both of the PI3K-AKT-mTOR and RAS-RAF-MAPK pathways, including receptor tyrosine kinases (EGFR and EGFRvIII), phosphotase (PTEN) and phosphorylated proteins (pAKT, pS6 and pERK) can be quantified in parallel with single-cell precision. Remarkably, the dynamic changes of those proteins upon the exposure to exogenous stimuli (e.g., growth factors and kinase inhibitors) can be kinetically monitored, providing a potent tool to better understand how glioblastoma cells respond to a combination of drug treatments. In parallel, Dr. Wu's research group has been studying the molecular mechanism of PTEN-controlled tumorigenesis. PTEN is the second most frequently deleted human tumor suppressor gene^{11-13,19,20}. The PTEN mutation was also found to be the cause of three autosomal dominant tumor predisposition syndromes. Dr. Wu's laboratory has generated various isogenic cell lines for pathway analyses and in-vivo tumor models for understanding the molecular and genetic mechanisms underlying PTEN controlled tumorigenesis in mice^{22,107-109}, Dr. Mischel's clinical pathology expertise bridges MIC technology to clinical applications.

### Semi-Automated MIC Technology

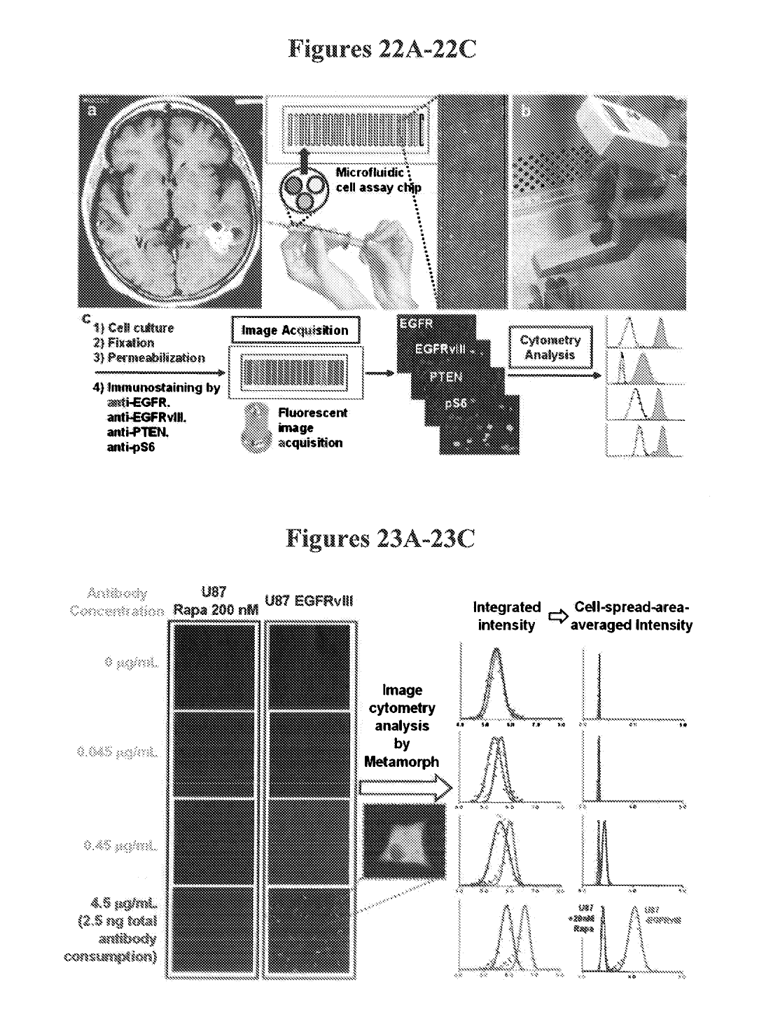

An embodiement of MIC technology (FIG. 22) has: a PDMS-based microfluidic cell array chip, a semi-automated pipette for performing cell seeding/culture and immunocytochemistry (ICC), an automated fluorescence microscope and associated software for image acquisition and analysis. The cell array chip is fabricated using soft lithography techniques, followed by O₂-plasma bonding or direct attachment using uncured PDMS films. A typical cell array chip accommodates a 24 cell culture chambers (with dimensions of 7000 um (l)×1000 um (w)×80 um (h) and total volume of 560 nL. Depending on the cell type, about 200 to 2000 cells can he accommodated in a single cell culture chamber. There is a pair of inlet and outlet channels located at either end of the chamber, allowing delivery of media and reagents to the cultured cells. The semi-automated pipettor directly inserts into an inlet and sample/solution volumes and flow rates are digitally controlled with precision. In our proof-of-concept studies, genetically and biochemically defined glioblastoma cells, including parental and genetically modified U87 cells (U87, U87-PTEN, U87-EGFR, U87-EGFRvIII and U87-EGFRvIII/PTEN) with overexpressed signaling proteins, as well as primary tumor tissues were tested to validate this embodiment of MIC technology. During the experiment, the cell array chips were placed in a humidified incubator (5% CO₂, 37° C.). PDMS is gas-permeable and rapid gas exchange is possible between the incubator environment and the cell culture media in the chambers.

FIG. 22 discusses a conceptual summary of the Microfluidic Image Cytometry (MIC) technology: (a) Dissociated glioblastoma cells obtained from a patient are introduced into a microfluidic cell array chip for multi-parameter analysis by MIC technology. (b) A semi-automated pipette executes cell seeding/culture and ICC. (c) The ICC-treated samples in the chips are mounted on a fluorescent microscope for image acquisition followed by analysis using an image cytometry program. (i,e., Metamorph, Molecular Devices Inc.) to quantify the expression levels of signaling proteins with single-cell resolution.

### Optimization of On-Chip Cell Culture Conditions and Quantification of Cell Growth

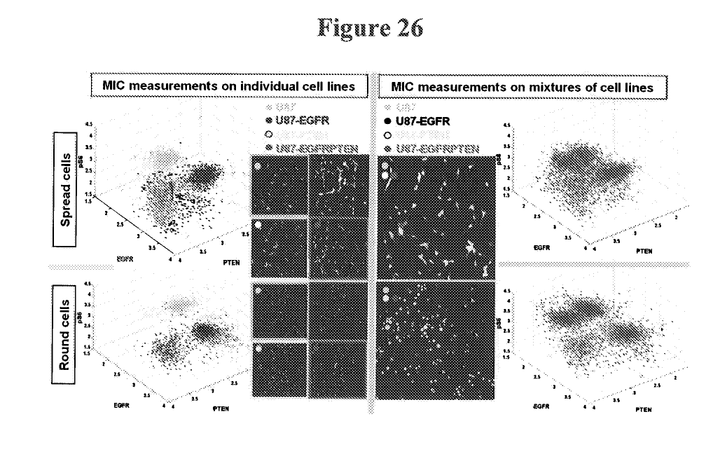

We used the cell array chips to determine the optimal surface coating, as well as the best cell feeding schedules to maintain the chip-cultured cells according to an embodiment of the current invention. A poly-L-lysine (PLL) coating on the glass/PDMS substrates was optimal for culturing parental and genetically modified U87 cell lines. For maintaining the primary glioblastoma cells dissociated from glioblastoma patient tissues, we developed a layer-by-layer coating method of Matrigel on the glass/PDMS surfaces. Further, we demonstrated that feeding cycles ranging between 12 and 36 hrs allowed for reproducible proliferation of the glioblastoma cells for at least 10 days. Viability assays (e.g.. Calcein AM or MitoTracker Red from Invitrogen) indicated cell viabilities in the devices. The growth rates of GBM were quantified by counting the cell numbers at different time points. Although inhibition of cell proliferation has been reported¹⁰³ in other microfluidic cell culture settings, the growth rates of the glioblastoma cells in the cell array chip were compatible with those observed in conventional dishes¹¹⁰. Additionally, we have also developed a variety of direct attachment approaches to immobilize freshly dissociated/released cells on to the substrates of cell array chips. We were able to deposit a layer of tissue adhesive protein (Cell Tak™) to immobilize cells from their suspension solutions enabling the MIC measurement of freshly dissociated cells (see FIGS. 26 and 28). We have also introduced streptavidin and DNA coatings onto the substrates our cell array chips, so that biotinylated and DNA-conjugated capture agents (e.g., can be surface-immobilized to facilitate chip-based cell sorting.

FIG. 26 shows parallel signaling profiling using the MIC technology. Four Signaling molecules, including EGFR, PTEN, pAKT and pS6 were detected and quantified in parallel using the four individual cell clines (LEFT, i.e., U87 (green), U87-EGFR (blue), U87-PTEN (yellow) and U87-EFGR/PTEN (pink)), as well as their mixtures (RIGHT). The MIC measurements were performed at two different conditions (i.e., before (BOTTOM) and after cell spread (TOP) in the MIC chips). Overlay micrographs of the five-color immunostaining (i.e., DAPI, anti-EGFR (Cy7), anti-PTEN (TRITC), anti-pAKT (Cy5) and anti-pS6 (FITC)) of the individual cell lines and cell mixtures were obtained (MIDDLE). After image cytometry analysis, 3-Diinensional (3D) scatter plots are utilized to display the power of this multi-parameter analysis—the four different types of cells are virtually dissected into four distinct groups in the space. The measurements of cell mixtures reproduce (RIGHT) the results obtained for the separate measurements (LEFT).

FIG. 28 shows two different view angles of a 3-D scatter plot were utilized to illustrate the cellular heterogeneity of a brain tumor sample analyzed in the MIC-chip. Three types of cells—blue circle (normal neuron cell), red circle (mono-lesion brain cancer cells) and green circle (dual lesion brain cancer cells)—can be identified in the plot.

### Quantitative Analysis of Chip-Based Immunocytochemistry (ICC) Using Overexpressed and Null Cells

In general, ICC uses various fluorophore-labeled antibodies to recognize specific protein molecules in cells. Due to high antibody reagent costs, it becomes impractical to carry out parametric assay optimization (reagent concentration, protocols . . . etc.) at the conventional macroscopic level. As a result, the ICC approach can only examine the presence/absence of target proteins in cells and the fluorescent signals in the ICC-treated samples do not reflect the absolute quantity of target proteins. The foundation of the MIC technology was built upon thoroughly optimized protocols that ensure reliable and reproducible ICC. In order to achieve measurement precision over a wide range of protein expression/phosphorylation level, we carried out systematic optimization of ICC protocols for each antibody. Five signaling proteins of interest associated with the PI3K-AKT-mTOR pathway—namely EGFR, EGFRvIII, PTEN, pAKT and pS6—were tested using the corresponding low/high-expression cell line systems¹¹⁰ . . . U87 vs. U87-EGFR, U87 vs. U87-EGFRvIII, U87 vs. U87-PTEN Triciribine-treated U87-EGFRvIII vs. EGFRvIII and rapamycin-treated U87 vs. U87-EGFRvIII, respectively. We were able to test a variety of ICC reagents and conditions for cellular fixation, membrane permeabilization and antibody staining, as well as the operation parameters of the semi-automated pipette for controlling the reagent/solution volumes and applied flow rates. The optimized condition utilizes 4% paraformaldehyde for cell fixation and 0.3% Triton X-100 for cell membrane permeabilization. The highly specific antibodies anti-EGFR (BD Pharmigen), anti-EGFRvIII (Dako), PTEN (Cascade), pAKT (Cell Signaling) and pS6 (Cell Signaling) was each covalently conjugated with a different fluorophore—emitting at 750 (Cy7), 647 (Cy5), 555 (TRITC), 647 (Cy5) and 488 (FITC) nm, respectively. Based on a similar approach, we were able to optimize a MIC protocol for pERK (a signaling node in the RAS-RAF-MAPK pathway, data not shown) using the respective controlled cell lines.

The MIC chip containing ICC-treated cells was mounted onto the fluorescent microscope (Nikon TE2000S) for image acquisition. Operational parameters of the microscope and CCD camera (Photomatrix. Cascade II), i.e., image exposure times and EM gains, were also optimized to attain superior signaling-to-noise ratios for the fluorescent images. The MetaMorph program (Molecular Devices) was used to quantify specific fluorescent signals in individual cells and generate cytometry histograms. In each set of measurements, a pair of histograms for both low and high-expression cell samples is generated after image analysis. The purpose of fine-tuning the MIC conditions and imaging parameters is to achieve maximum separation of the two histograms. Good separation becomes critical for quantifying multiple signaling proteins. FIG. 23 displays the fine-tuning process of the FITC-labeled pS6 antibody concentrations to obtain the optimal dynamic range for quantification of pS6 phosphorylation in U87-EGFRvIII cells (with consistently high pS6 levels) and rapamycin-treated U87 cells (with low pS6 levels, as a result of mTOR inhibition by the rapamycin). We found that an anti-pS6 concentration of 4.5 μg/mL gave the best contrast of fluorescence signals between the pS6-positive and negative cells. Each ICC experiment used only 700 nL of pS6 antibody solution (ca. 2.5 ng of antibody), demonstrating at least two orders of sample economy compared to conventional ICC approach on microscope cover slides. Additionally, the quantification of signaling molecules is highly reproducible. Besides the pS6 quantification, optimal ICC protocols for other signaling proteins, including EGFRvIII, EGFR PTEN and pAKT, were obtained through similar optimization processes.

FIG. 23 shows optimization of the concentrations of FITC-labeled pS6 antibody for quantifiable ICC. a) Micrographs obtained for the U87-EGFRvIII (pS6+) and rapamycin-treated U87 (pS6−) in the presence of different anti-pS6 concentrations (i.e., 0, 0.045, 0.45 and 4.5 ug/mL). b) MetaMorph was used for image cytometry analysis, indicating that the antibody concentration of 4.5 ug/mL generated the best separation between the pS6 positive and negative cells. c) Cell-spread-surface-area average and background subtraction improved the peak separation and reduced the bandwidth (spread) of the histograms.

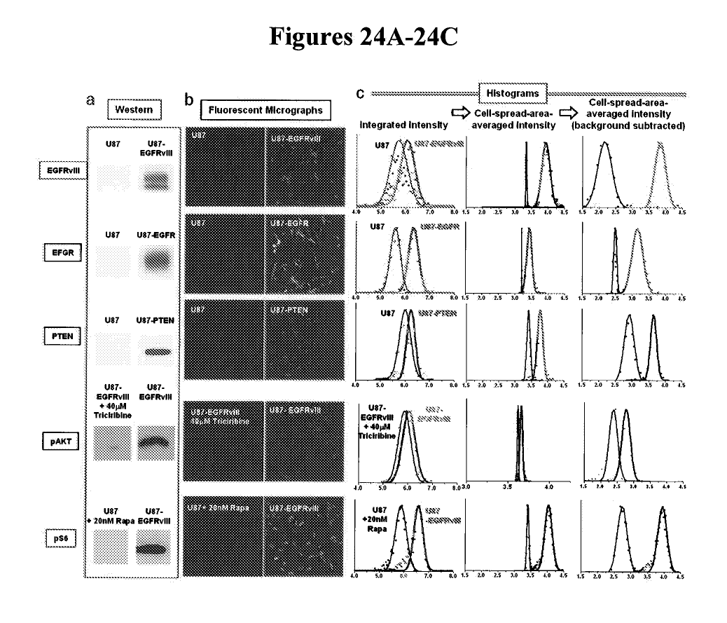

FIG. 24 summarizes the dynamic ranges for detecting of EGFRVIII, EGFR, PTEN, pAKT and pS6 in individual cells using their optimal ICC conditions and operation parameters, These results were validated by Western blot measurements using of identical cell samples. By applying (i) cell-spread-surface-area average (to rule out variation in cell sizes) and (ii) background subtraction, the separation between each pair of histograms is improved and the band width of each histogram is sharpened, reflecting significantly improved dynamic range and sensitivity of the MIC technology.

FIG. 24 shows dynamic ranges of the MIC technology for single-cell profiling of EGFRvIII, EGFR, PTEN, pAKT and pS6 under the optimal ICC conditions. a) Western data for detecting the five signaling proteins in the control cell lines. b) Fluorescent micrographs of the ICC-treated cells. c) The improvement of the dynamic ranges for quantification of the five signaling proteins in individual cells. Both cell-spread-area-averaged and background subtraction enhance the dynamic range for quantification.

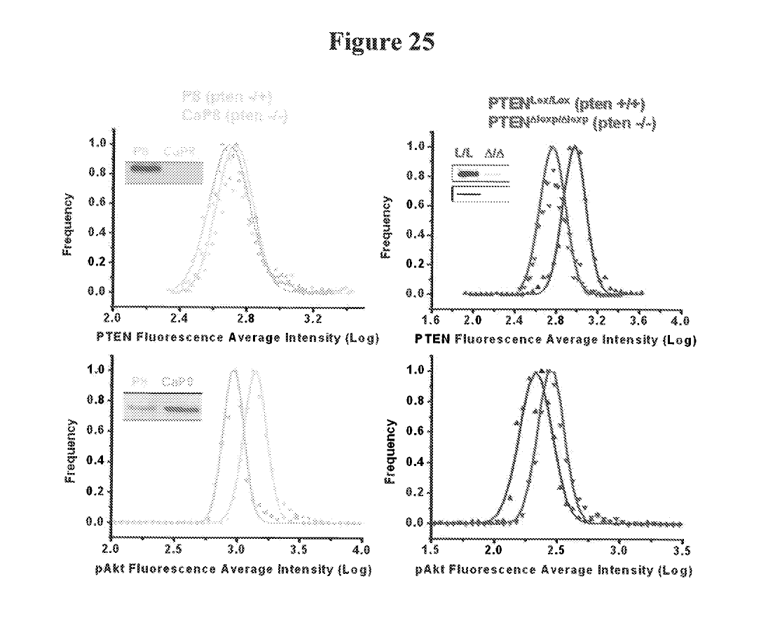

One of the major challenges in defining genetic lesions is measuring the loss of heterozygosity (LOH) of a particular tumor suppressor gene (e.g., PTEN) since (i) the intrinsic signals in the tissues are relatively low and (ii) the surrounding normal tissues express a normal level of tumor suppress gene, generating a significant background signal, PTEN is the second most frequently deleted tumor suppressor gene found in human cancers, and PTEN negatively regulates the PI3K-AKT-mTOR pathway. To test the feasibility of quantifying PTEN expression in primary cells (where PTEN expression levels are at 2, 1 or 0) using the MIC technology, two sets of isogenic mouse cell lines, including (i) PTEN ES lines (i.e., p8 (+/−) and CaP8 (−/−) and (ii) MEF lines (i.e,, PTEN^{loxp/loxp} (+/+) and PTEN^{Δloxp/Δloxp} (−/−). As shown in FIG. 25, the MIC technology generated two pairs of histograms, indicating that the MIC technology was able to distinguish both heterozygosity (LOH, and complete PTEN deletion in the respective sample sets (i.e., p8 (+/−) vs. CaP8 (−/−) and PTEN^{loxp/loxp} (+/+) vs. PTEN^{Δloxp/Δloxp} (−/−)). Using the same batch of cell samples, the results obtained by the MIC technology are consistent with those observed by Western blot. MIC measurement consumed only 2000 cells for each set of measurement, while 10⁶ cells are required to achieve reliable signals. PTEN downstream signaling node, pAKT can also be detected by the MIC technology, and the results indicated that PTEN negatively regulates the PI3K-AKT-mTOR pathway.

FIG. 25 shows quantification of PTEN expression and pAKT phosphorylation of two sets of isogenic mouse cell lines, including (i) PTEN ES lines, i.e., p8 (+/−) and CaP8 and (−/−), and (ii) MEF lines, i.e. PTEN^{loxp/loxp} (+/+) and PTEN^{Δloxp/Δloxp} (−/−). Western blots and the MIC measurements were carried out in parallel using 10⁶ and 2000 cells, respectively. The MIC technology was able to distinguish both loss of heterozygosity (LOH, p8 (+/−) vs. CaP8 (−/−)) and complete PTEN deletion (PTEN^{loxp/loxp} (+/+) vs. PTEN^{Δloxp/Δloxp} (−/−)). The results obtained from the MIC technology are consistent with those observed by Western Blot. The PTEN downstream signaling node, pAKT is also detected by the MC technology and the results indicate that PTEN negatively regulates the PI3K-AKT-mTOR pathway.

### Parallel Detection of Four Signaling Molecules in a Cell Mixture Composed of Four Cell Types

The MIC technology is capable of parallel detection of several signaling molecules in individual cells enabling the capture of cellular heterogeneity in a tissue sample. To prove this, we carried out (FIG. 26) parallel detection of the four signaling molecules (i.e., EGFR, PTEN, pAKT and pS6) using individual cell lines (i.e., U87, U87-EGFR, U87-PTEN and U87-EGFR/PTEN) and an artificial cell mixture (containing the four cell lines in a 1:1:1:1 ratio). The MIC measurements were performed at two different conditions (i.e., before and after cell spreading to compared how cell morphologies affect the signaling profiles. The individually developed ICC conditions were combined, so that the paraformaldehyde-fixed and detergent-permeabilized cell mixture in the microchip was treated with an antibody cocktail containing Cy7-labeled anti-EFGR, Cy5-labeled anti-pAKT, TRITC-labeled anti-PTEN, and FITC-labeled-pS6, yielding a multi-color stain (DAPI, anti-EGFR (Cy7), anti-PTEN (TRITC), anti-pAKT (Cy5) and anti-pS6 (FITC)). Images collected at diffitrent fluorescent channels were merged to show co-localization of the four signaling molecules. By performing image cytometry analysis, the EGFR, PTEN, pAKT and pS6 levels in individual cells were obtained. 3-dimensional (3D) scatter plots (with the X, Y and Z axes representing EGFR, PTEN and pS6, respectively) distinguish the different cell types by multi-parameter analyses. As shown in FIG. 26, the four different cell types are virtually dissected into four distinct groups. Simultaneously, we also tested a mild cell detachment approach: 15-min TryPEL Express (Invitrogen) treatment at 37° C., to harvest cells from culture flasks with minimum impact to cell signaling events. Using this treatment, freshly detached cells were immobilized onto the microfluidic channels coated with the adhesion matrix (Cell-Tak™). The sequential MIC measurements revealed that the attached “round cells” gave very similar signaling profiled to those observed for the stabilized cells with well-spread morphology. The mild cell detachment approach was applied for dissociated of surgically removed brain tumor tissues with minimum impact on the signaling profiles of the primary cells.

### Single-Cell Pharmacodynamic Study on Rapamycin Using MIC Technology

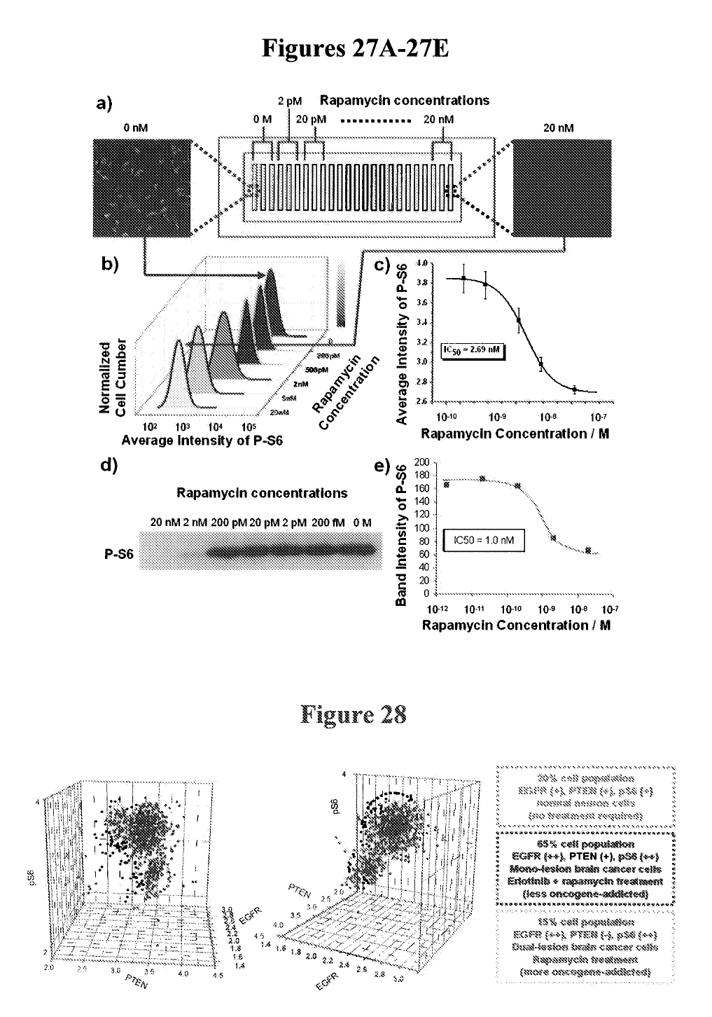

The ICC approach is often used to examine the presence/absence of target proteins in cells, but due to poor data reproducibility it is not suitable for quantification of protein expression/phospohorylation. Given the ability of MIC technology to perform quantification as described above, we attempted to apply MIC technology for determination of rapamycin pharmacodynamics. Rapamycin is a potent inhibitor targeting mTOR, a critical signaling molecule in the PI3K signaling pathway. The effect of rapamycin inhibition on mTOR can be quantified by monitoring the activation of the downstream signaling molecule pS6. The MIC technology was used to quantify rapamycin-induced down-regulation of pS6 levels in individual cells (FIG. 27) in this case, U87 cells were cultured in a microfluidic cell array chip, where cell culture media containing different concentrations (i.e., 0, 0.2, 0.5, 2.0, 5.0 and 20 nM) of rapamycin were introduced into different sets of cell culture chambers. The resulting histograms that were comnosed of thousands of single-cell data indeed reflect the dose-dependent inhibition. A dose-dependent curve can be obtained by plotting averaged pS6 levels vs. the corresponding rapamycin concentrations, generating an IC₅₀ of 2.69 nM. This IC₅₀ was validated by Western blot-based quantification (1.0 nM) in which 10⁶ cells were required for each data point.

FIG. 27 shows quantification of pS6 expression levels in individual cell treated with different concentrations of rapamycin. a) U87 cells were cultured in a microfluidic cell array chip, where cell culture media contained 0, 0.2, 0,5, 2.0, 5.0, 20 nM of rapamycin. b) The rapamycin-induced down-regulation of pS6 levels in individual cells were quantified by the MIC technology. Six histograms corresponding to different dose of rapamycin reflect the different levels of rapamycin inhibition. c) A dose-dependent curve can be obtained by plotting pS6 levels vs. the corresponding rapamycin concentrations. IC₅₀ (2.69 nM) was deduced from the dose-dependent curve. The IC₅₀ measured by MIC technology was validated by Western blot using 10⁶ for each data point. The resulting dose dependent curve gives an IC₅₀ of 1.0 nM.

### Clinical Validation of the MIC Technology Using Brain Tumor Tissue

The successful demonstration of MIC technology using glioblastoma cell lines encouraged us to test the MIC in-vitro molecular diagnostic technology in clinical setting. We were able to bond our first validation study with an existing clinical trial focused on brain tumor targeted therapy. A group of clinically well-characterized brain cancer patients at UCLA medical school were recruited and tested. Brain tumors often contain high percentages of cancer cells, thus are an outstanding model system for validating the MIC technology. So far, we were able to carry out the quantitative/multi-parameter ICC in the MIC chips to measure molecular signatures (i.e. EGFR, EGFRvIII, PTEN, pAKT and pS6) on seven surgically removed brain tumors (i.e., glioma at different grades). To ensure a minimum level of perturbation to the cells, a mild and rapid tumor dissociation protocol developed by our joint team (15-min TryPEL Express treatment at 37° C.) was established to process freshly isolated tumor tissues. As shown in FIG. 28, three subsets of cells were identified from a pediatric brain tumor tissue, which reflect intrinsic tissue heterogeneity of the tumors as well as characteristics of different cell types. For those patients who received treatment of targeted drug (e.g., rapamycin), dramatically reduced pS6 phosphorylation levels in their tumor cells were observed (data not shown), indicating the therapy-induced signaling inhibition. Currently, the MIC-chips are involved in further validation with human prostate cancer and bladder cancer, as well as leukemia cell lines and other patient samples.

### Automated Sample Preparation/Immunostaining—Creating a User-Friendly Interface Between Microfluidic Cell Array Chips and a Robotic Pipetting System in Order to Enable Large-Scale Signaling Profiling in a Closely-Related Microenvironment

The original MIC platform utilized a human-operated semi-automated pipette to perform sample preparation and ICC in sequence. The pipette digitally controls the flow rates and volumes, but the rest of the operation/process was, in fact, manually operated. Generally, human involvement introduces operator variability and error, therefore the semi automated MIC platform requires scale-up for applications requiring high-throughput and/or multistep studies. A user-friendly interface between the microfluidic cell array chip and a robotic pipette (FIG. 28A-28B) has been developed. Our goal is to establish a fully automated operation from initial cell loading and culturing (with media exchange) to immunostaining, so that a complicated large-scale cell culture/assay can be carried out for high-throughput signaling network profiling.

### Design of Robotic Pipette for Automated Sample Preparation and Immunostaining

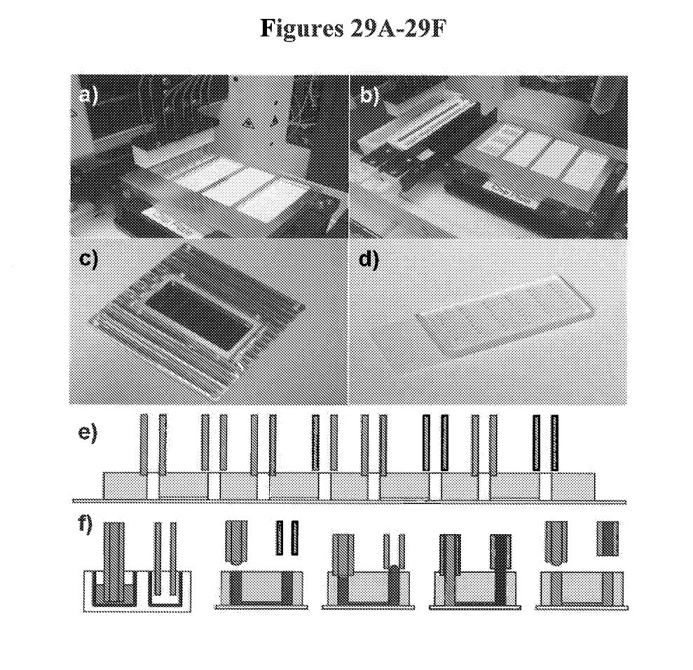

The mechanical aspect of the user-friendly interface is composed of two custom-designed components, i.e., a chip holder (FIG. 29A-29B) and a pipette tip array (FIG. 29A-photo and 29E-schematic). The robotic pipetting system (Nanodrop II, Innovadyne) is designed for handling 96-, 384- and/or 1536-well plates. To match these standard configurations, the chip holder was designed to have the same footprint as a well plate to enable immediate interfacing with the robotic system (and other well plate systems). Currently, one chip holder can accommodate four microfluidic cell culture chips, each with 30-40 cell culture chambers. The locations of individual inlet and outlet holes of the cell culture chambers register to the positions of wells in a 1536-well plate. Therefore, the original program developed for handling 1536-well plates can be simply optimized for performing sample preparation and ICC, on MIC chips.

FIG. 29 shows a robotic pipetting system for performing large-scale signaling profiling in an automated fashion according to an embodiment of the current invention, a) The robotic pipetting system designed for handling samples and reagents within a well-plate format. b) A custom-designed chip holder the same footprint as a well plate allows convenient and precise interface between the robotic system and cell array chips. c) A custom-designed mold for preparation of the chips with a fixed standardized chip height. d) A cell array chip with 30 cell culture chambers. e) A cross section of a chip fitted with four pairs of pipettes. f) Schematic representation illustrates how a pair of pipette tips is utilized for dispensing and withdrawing sample/solution in a microfluidic cell culture chamber.

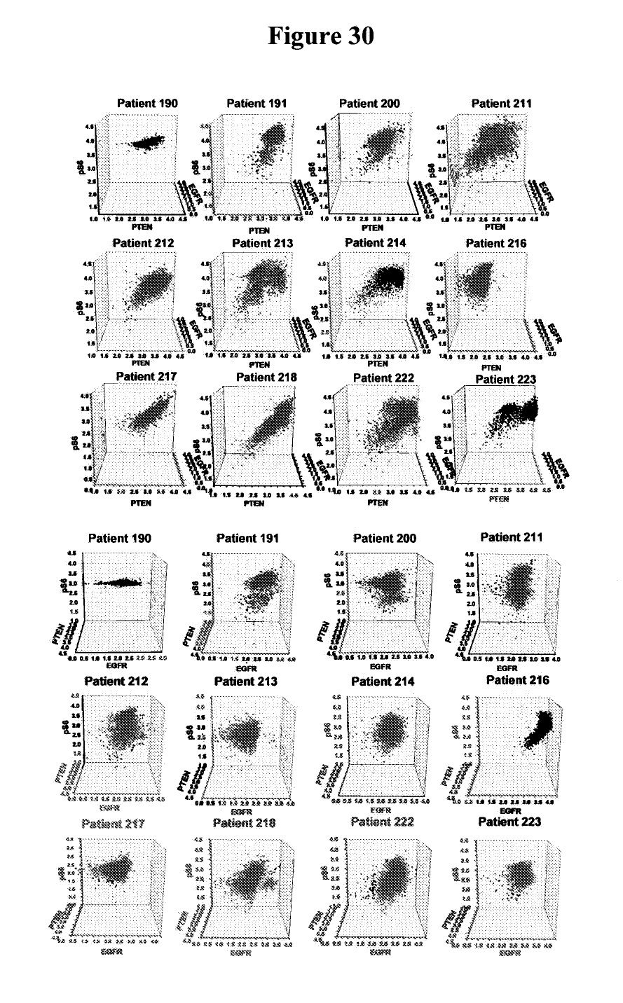

FIG. 30 shows 3-D scatter plots of 12 brain tumor samples analyzed in the MIC-chip 12, revealing dramatically different cellular heterogeneity of individual tumor samples.

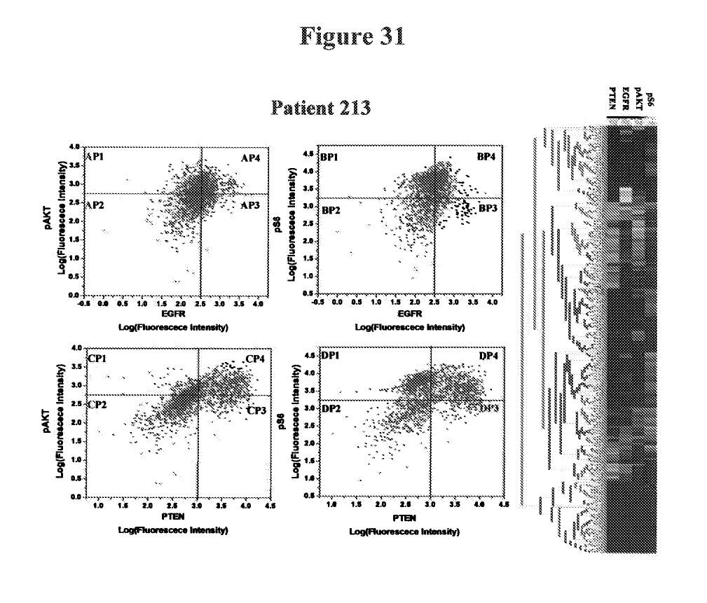

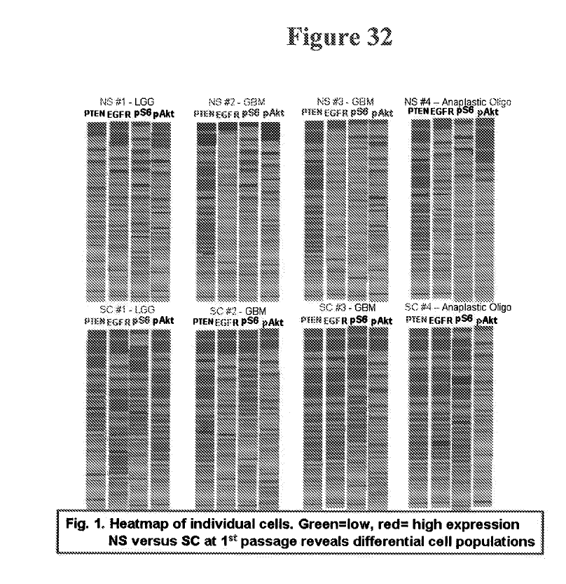

FIG. 31 shows heretical clustering approach that was employed to analyze and quantify cellular heterogeneity of a given patient samples

### Design of Chip-Holder and Alignment System

The chip holder is a key component that can ensure precise position and orientation of the microfluidic chips with respect to the coordinate system of the robotic pipetting system. Pipetting tips are precisely aligned with inlets and outlets of cell culture chambers. A combination of mechanical depressions for the chips and a clamping mechanism ensure convenient and consistent positioning of the chips with tolerances for slight differences in assembly.

The robotic pipetting system has eight individually controlled pipette tips, each capable of dispensing and withdrawing liquid samples with 5-nL precision. To integrate the MIC chip with the robotic pipetting system, we grouped the eight tips into four pairs for handling four cell culture chambers (FIG. 29E). In each tip pair, one is assigned to dispense fluid, and the other for collecting “exhaust” fluid. Our design exploits the elasticity of the PDMS-based microfluidic chip to achieve a liquid-tight seal between each pipette tip and the inlets of the microfluidic chip to eliminate challenging bubble issues in the microfluidic chambers. The tip diameter is roughly 1.0 mm and the diameter of the PDMS holes are about 400 μm, allowing reasonable tolerance for positioning errors. FIG. 29F illustrates how a pair of tips handles simultaneous sample/solution loading and removal in a microfluidic cell culture chamber. The left tip aspirates new media or reagent from a reagent reservoir and then seals to the top surface of the PDMS chip above the inlet hole. Liquid is injected into the channel and pushed out of the outlet. This waste solution is withdrawn by the second tip. In contrast to the semi-automated pipette approach which takes about 20 minutes to complete a routine cell culture medium exchange on a chip, the same process can be completed in less than 10 seconds with this robotic system. A special mold was designed and fabricated (FIG. 29C) to mass produce chips with consistent dimension because the chip thickness is integral to the liquid-tight seal formation.

### User-Friendly Control Interface

In addition to the mechanical aspects of the interface, there is also an important software component. We have developed a very flexible program interface that implements generic “protocols/recipes” (cell loading, media exchange, ICC, etc . . . ) composed of standardized steps configured in an XML file. Each protocol step specifies a reagent location, dispensing volume, dispensing speed, etc. The user simply selects the recipe to automatically execute. The software allows specification of which chips (and which columns per chip) should be loaded with the new media/reagent for more complex studies involving different cell types or different treatments.

### Clinical Validation Based on Tissue Samples Obtained from 20 Brain Tumor Patients

A group of clinically well-characterized brain cancer patients at UCLA medical school were recruited and tested. Brain tumors often contain high percentages of cancer cells, thus are an outstanding model system for validating the MIC technology. So far, we were able to carry out the quantitative/multi-parameter ICC in the M1C chips to measure molecular signatures (i.e. EGFR, EGFRvIII, PTEN, pAKT and pS6) on twenty surgically removed brain tumors (i.e., glioma at different grades). To ensure a minimum level of perturbation to the cells, a mild and rapid tumor dissociation protocol developed by our joint team (15-min TryPEL Express treatment at 37° C.) was established to process freshly isolated tumor tissues. 3D logarithmic scatter plots were used to visualize the protein expression and phosphorylation level in each cell. Each patient has different plots distribution and clustering, which reflect intrinsic tissue heterogeneity of the tumors as well as characteristics of different cell types. FIG. 30 shows the results obtained from 12 brain tumor patients.

Dual Parameter Dot Plots have been used to display the relationship of the upstream proteins, PTEN and EGFR, with the downstream proteins, pAKT and pS6. The thresholds for high expression and low expression of PTEN, EGFR, pAKT and pS6 were obtained through the statistic of all the patient data. We also can obtain the percentage of cells located in each subset. A hierarchical clustering shows the four protein expression level at the same time and the similarity of each subset cells.

### Example

### Development of Microfluidic-Based Image Cytometry(MIC) for Molecular Analysis of Brain Tumors and Brain Tumor Stem Cells

Data suggests that an in vitro neural stem cell (NSC) culture system composed of serum-free media supplemented with epidermal growth factor and fibroblast growth factor, when used to culture human primary tumors isolates, enriches and maintains a tumor-initiating/tumorigenic population of cells termed ‘brain tumor stem cells’ (BTSCs). These cells express neural stem cell genes (Nakano, et al.), have properties of surface attachment-free growth and form characteristic spheroid aggregates called ‘neurospheres’. Unlike their ‘non-stem’ attachment-dependent counterparts that are enriched with an in vitro culture system consisting of a serum-based media, these putative BTSCs fulfill major tenets of cancer stem cell biology in that they are self-renewing, capable of multi-potential differentiation and, when xenotransplanted, form tumors that recapitulate many aspects of the parental tumor they were derived from. Recent data suggests that the growth and ability for patient BTSC lines to be maintained over long-term passages mimic the clinical progression of the patient tumors and thus, this in vitro model serves as an independent prognostic factor (Laks, et al.).

Another component of BTSC theory, derived from the A,B and C-type neural progenitors found in normal neural development, is that these BTSCs may be more quiescent and less mitotic active than other brain tumor cellular progeny, with the most quiescent cells retaining the most ‘stem-like’ characteristics and rapidly-dividing cells having a more limited progenitor capacity. This has been posited as one of the reasons these cells are ‘chemo-resistant’ simply because, until the advent of classes of drugs known to target molecules that specifically attenuate a cell-signaling pathway, most common chemotherapeutic modality used clinically targeted cells with high mitotic activity.

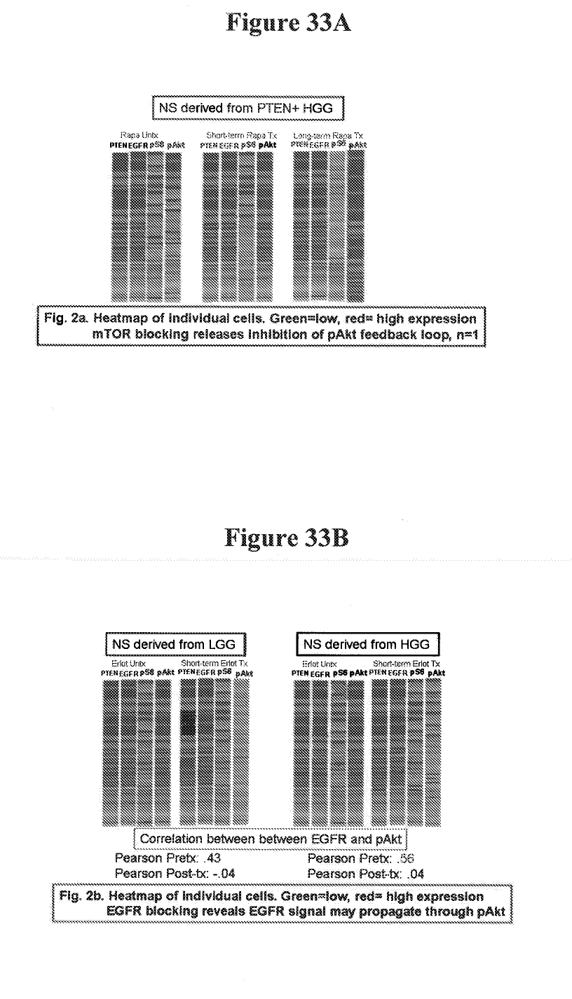

The signaling pathway of interest as the proof-of-concept for the microfludic-based image cytometry (MIC) system was the phosphoinosioI 3 kinase (PI3-K) pathway since its dysregulation is implicated in both brain tumor oncogenesis and brain cancer stem cell evolution and maintenance. Simultaneous multi-nodal (pS6, PTEN, pAkt and EGFR) analysis with single cell resolution allows the study of these components in each cell in primary tumors, cultured brain tumor stem cell and non-stem cell progeny. The MIC system is picking up valuable intra and inter-group pathway differences and the identity of personalized BTSC molecular ‘signatures’ via comparison of patient brain tumor samples with their non-BTSC and BTSC-enriched progeny (at the very 1^{st} in vitro passage) has revealed striking pathway similarities to distinguish stem (NS, ‘neurosphere’) and non-stem (SC, ‘serum cell’). Although the significance is still not clear, most notable quantitative shifts observed are lower expression of EGFR and pS6 in NS samples versus SC samples at the single cell level (FIG. 32).

MIC-derived analysis of chemo-sensitive and chemo-resistance phenomena is yielding data which may allow further detailed characterization of BTSCs based on pathway response to chemotherapy. For instance, at least 2 novel chemotherapeutic phenomena are emerging in patient BTSC lines and this is even irrespective of the parental tumor's World Health Organization (WHO) clinical diagnosis. The first is the rapamycin-induced chemo-activation due to the release of mTOR's feedback inhibition on activated pAkt at the single cell level (FIG. 33A). A recent UCLA Phase I clinical trial of rapamycin in GBMs was recently completed revealing that this phenomenon occurred in half of the clinical cohort.

Additionally, in tests of multiple BTSC lines, a new predictive model for chemotherapeutic response reveals that signal propagation from EGFR is through Akt. The predictive power of this model initially came from the observation that when EGFR expression significantly correlates with pAkt in pretreated samples, EGFR blocking abrogates the correlation post-treatment and can quantitatively reduce activated Akt expression at the single cell level (FIG. 33B). This implication activated Akt with respect to these 2 phenomena is of special interest to the BTSC biological community since Akt signaling is thought to be profoundly important for maintaining the malignant stem cell phenotype.

The invention has been described in detail with respect to various embodiments, and it will now be apparent from the foregoing to those skilled in the art that changes and modifications may be made without departing from the invention in its broader aspects, and the invention, therefore, as defined in the claims is intended to cover all such changes and modifications as fall within the true spirit of the invention.

### REFERENCES—DETAILED DESCRIPTION SECTION

1. Kleihues, P. & Ohgaki, H. Phenotype vs genotype in the evolution of astrocytic brain tumors. Toxicologic pathology 28, 164-170 (2000).

2. von Deimling. A. et al. Subsets of glioblastoma multiforme defined by molecular genetic analysis. Brain pathology (Zurich, Switzerland) 3, 19-26 (1993).

3. Lang. F. F., Miller, D. C., Koslow, M. & Newcomb, E. W. Pathways leading to glioblastoma multiforme: a molecular analysis of genetic alterations in 65 astrocytic tumors. Journal of neurosurgery 81, 427-436 (1994).

4. Kleihues, P. & Ohgaki, H. Primary and secondary glioblastomas: from concept to clinical diagnosis. Neuro-oncology 1, 44-51 (1999).

5. Louis, D. N., Holland, E. C. & Cairncross, J. G. Glioma classification: a molecular reappraisal. The American journal of pathology 159, 779-786 (2001).

6. Rich, J. N. et al. Phase II trial of gefitinib in recurrent glioblastoma. J Clin Oncol 22, 133-142 (2004).

7. Barber. T. D., Vogelstein, B., Kinzler, K. W. & Velculescu, V. E. Somatic mutations of EGFR in colorectal cancers and glioblastomas. The New England journal of medicine 351, 2883 (2004).

8. Marie, Y. et al. EGFR tyrosine kinase domain mutations in human gliomas. Neurology 64, 1444-1445 (2005).

9. Rich, J. N., Rasheed, B. K. & Yan, H. EGFR mutations and sensitivity to gefitinib. The New England journal of medicine 351, 1260-1261; author reply 1260-1261 (2004).

10. Smith, J. S. et al. PTEN mutation, EGFR amplification, and outcome in patients with anaplastic astrocytoma and glioblastoma multiforme. Journal of the National Cancer Institute 93, 1246-1256 (2001).

11. Aldape, K. D. et al. Immunohistochemical detection of EGFRvIII in high malignancy grade astrocytomas and evaluation of prognostic significance. Journal of neuropathology and experimental neurology 63, 700-707 (2004).

12. Ekstrand. A. J. et al. Genes for epidermal growth factor receptor, transforming growth factor alpha, and epidermal growth factor and their expression in human gliomas in vivo. Cancer research 51, 2164-2172 (1991).

13. Frederick, L.. Wang, X. Y., Eley, G. & James. C. D. Diversity and frequency of epidermal growth factor receptor mutations in human glioblastomas. Cancer research 60, 1383-1387 (2000).

14. Sugawa, N., Ekstrand, A. J., James. C. D. V. P. Identical splicing of aberrant epidermal growth factor receptor transcripts from amplified rearranged genes in human glioblastomas. Proceedings of the National Academy of Sciences of the United States of America 87, 8602-8606 (1990).

15. Wong. Al et al. Structural alterations of the epidermal growth factor receptor gene in human gliomas. Proceedings of the National Academy of Sciences of the United States of America 89, 2965-2969 (1992).

16. Batra, S. K. et al. Epidermal growth factor ligand-independent, unregulated, cell-transforming potential of a naturally occurring human mutant EGFRvIII gene. Cell Growth Differ 6, 1251-1259 (1995).

17. Choe, G. et al. Analysis of the phosphatidylinositol 3′-kinase signaling pathway in glioblastoma patients in vivo. Cancer research 63, 2742-2746 (2003).

18. Huang, H. S. et al. The enhanced tumorigenic activity of a mutant epidermal growth factor receptor common in human cancers is mediated by threshold levels of constitutive tyrosine phosphorylation and unattenuated signaling. The Journal of biological chemistry 272, 2927-2935 (1997).

19. Li, B. et al. Mutant epidermal growth factor receptor displays increased signaling through the phosphatidylinositol-3 kinase/AKT pathway and promotes radioresistance in cells of astrocytic origin. Oncogene 23, 4594-4602 (2004).

20. Sordella, R., Bell, D. W., Haber, D. A. & Settleman, J. Gefitinib-sensitizing EGFR mutations in lung cancer activate anti-apoptotic pathways. Science 305, 1163-1167 (2004).

21. Weinstein, I. B. Cancer. Addiction to oncogenes—the Achilles heal of cancer. Science 297, 63-64 (2002).

22. Ermoian, R. P. et al. Dysregulation of PTEN and protein kinase B is associated with glioma histology and patient survival. Clin Cancer Res 8, 1100-1106 (2002).

23. Bianco, R. et al. Loss of PTEN/MMAC1/TEP in EGF receptor-expressing tumor cells counteracts the antitumor action of EGFR tyrosine kinase inhibitors. Oncogene 22, 2812-2822 (2003).

24. Stephens, P. et al. Lung cancer: intragenic ERBB2 kinase mutations in tumours. Nature 431, 525-526 (2004),

25. Hay, N. The Akt-mTOR tango and its relevance to cancer. Cancer cell 8, 179-183 (2005).

26. Nave, B. T., Ouwens, M., Withers, D. j., Alessi, D. R., & Shepherd, P. R. Mammalian target of rapamycin is a direct target for protein kinase B: identification of a convergence point for opposing effects of insulin and amino-acid deficiency on protein translation. The Biochemical journal 344 Pt 2, 427-431 (1999).

27. Scott, P. H., Brunn G. J., Kohn. A. D., Roth, R. A. &. Lawrence, J. C., Jr. Evidence of insulin-stimulated phosphorylation and activation of the mammalian target of rapamycin mediated by a protein kinase B signaling pathway. Proceedings of the Motional Academy of Sciences of the United States of America 95, 7772-7777 (1998).

28. Fry, M. J. Phosphoinositide 3-kinase signalling in breast cancer: how big a role might it play? Breast cancer Res 3, 304-312 (2001).

29. Hu, Q., Klippel, A., Muslin. A.J., Fantl, W.J. & Williams, L. T. Ras-dependent induction of cellular responses by constitutively active phosphatidylinositol-3 kinase. Science 268, 100-102 (1995).

30. Lee. A. V. & Yee, D. insulin-like growth factors and breast cancer. Biomedicine & pharmacotherapy=Biomedecine & pharmacotherapie 49, 415-421 (1995).

31. Scheid, M. P. & Woodgett, J.R. Phosphatidylinositol 3 kinase signaling in mammary tumorigenesis. Journal of mammary gland biology and neoplasia 6, 83-99 (2001).

32. Delbeke, D. & Martin. W. H. PET and PET-CT for evaluation of colorectal carcinoma. Seminars in nuclear medicine 34, 209-223 (2004).

33. Dimitrakopoulou-Strauss. A. et al. Prognostic aspects of 18F-FDG PET kinetics in patients with metastatic colorectal carcinoma receiving FOLFOX chemotherapy. J Nucl Med 45, 1480-1487 (2004).

34. Truant, S. et al. Prospective evaluation of the impact of [18F]fluoro-2-deoxy-D-glucose positron emission tomography of resectable colorectal liver metastases. The British journal of surgery 92, 362-369 (2005).

35. Rohren, E. M. et al. The role of F-18 FDG positron emission tomography in preoperative assessment of the liver in patients being considered for curative resection of hepatic metastases from colorectal cancer. Clinical nuclear medicine 27, 550-555 (2002).

36. Wong. C. Y. et al. Metabolic response after intraarterial 90Y-glass microsphere treatment for colorectal liver metastases: comparison of quantitative and visual analyses by 18F-FDG PET. J Nucl Med 45, 1892-1897 (2004).

37. Rohren, E. M., Turkington. T. G. & Coleman, R. E. Clinical applications of PET in oncology. Radiology 231, 305-332 (2004).

38. Bos, R. et al. Biologic correlates of (18)fluorodeoxyglucose uptake in human breast cancer measured by positron emission tomography. J Clin Oncol 20, 379-387 (2002).

39. Chung, J. K. et al. Comparison of [18F]fluorodeoxyglucose uptake with glucose transporter-1 expression and proliferation rate in human glioma and non-small-cell lung cancer. Nuclear medicine communications 25, 11-17 (2004).

40. Kurokawa. T. et al. Expression of GLUT-1 glucose transfer, cellular proliferation activity and grade of tumor correlate with [F-18]-fluorodeoxyglucose uptake by positron emission tomography in epithelial tumors of the ovary. International journal of cancer 109, 926-932 (2004).

41. Pugachev. A. et al. Dependence of FDG uptake on tumor microenvironment. International journal of radiation oncology, biology, physics 62, 545-553 (2005).

42. Rajendran, J. G. et al. Hypoxia and glucose metabolism in malignant tumors: evaluation by [18F]fluoromisonidazole and [18F]fluorodeoxyglucose positron emission tomography imaging. Clin Cancer Res 10, 2245-2252 (2004).

43. Kato, H. et al. Correlation of 18-F-fluorodeoxyglucose (FDG) accumulation with glucose transporter (Glut-1) expression in esophageal squamous cell carcinoma. Anticancer research 23, 3263-3272 (2003).

44. Koga, H. et al. Differential FDG accumulation associated with GLUT-1 expression in a patient with lymphoma. Annals of nuclear medicine 17, 327-331 (2003).

45. Yen. T. C. et al. 18F-FDG uptake in squamous cell carcinoma of the cervix is correlated with glucose transporter 1 expression. J Nucl Med 45, 22-29 (2004).

46. Sankhala, K. K. et al. Early response evaluation of therapy with AP23573 (an mTOR inhibitor) in sarcoma using [18F]-fluoro-2-deoxy-D-glucose (FDG) positron emission tomography (PET) scan. J Clin Oncol 23, 9028 (2005).

47. Park. T. H. & Shuler, M. L. Integration of cell culture and microfabrication technology. Biotechnol Prog 19, 243-253 (2003).

48. Fu. A. Y., Chou, H. P., Spence. C., Arnold. F. H. & Quake, S. R. An integrated microfabricated cell sorter. Anal Chem 74, 2451-2457 (2002).

49. Xia, Y. N. & Whitesides, G. M. Soft lithography. Angewandte Chemie-International Edition 37, 551-575 (1998).

50. Quake, S. R. & Scherer. A. From micro- to nanofabrication with soft materials. Science 290, 1536-1540 (2000).

51. Unger, M. A., Chou, H. P., Thorsen. T., Scherer. A. & Quake, S. R. Monolithic microfabricated valves and pumps by multilayer soft lithography. Science 288, 113-116 (2000).

52. Elowitz, M. B., Levine. A. J., Siggia, E. D. & Swain, P. S. Stochastic gene expression in a single cell. Science 297, 1183-1186 (2002).

53. Raser, J. M. & O'Shea., Control of stochasticity in eukaryotic gene expression. Science 304, 1811-1814 (2004).

54. Coleman-Lerner. A. et al. Regulated cell-to-cell variation in a cell-fate decision system. Nature 437, 699-706 (2005).

55. Pedraza, J. M. & van Oudenaarden, A. Noise propagation in gene networks. Science 307, 1965-1969 (2005).

56. Rosenfeld, N., Young, J. W., Alon, U., Swain, P. S. & Elowitz, M. B. Gene regulation at the single-cell level. Science 307, 1962-1965 (2005).

57. George, T. C. et al. Distinguishing modes of cell death using the ImageStream multispectral imaging flow cytometer. Cytometry A 59, 237-245 (2004).

58. Burns N. et al., Large-scale analysis of gene expression, protein localization, and gene disruption in Saccharomyees cerevisiae. Genes & development 8, 1087-1105 (1994).

59. Eowitz, M. B. & Leibler, S. A, synthetic oscillatory network of transcriptional regulators. Nature 403, 335-338 (2000).

60. Huh, W. K. et al. Global analysis of protein localization in budding yeast. Nature 425, 686-691 (2003).

61. Kahana, J. A., Schnapp, B. J. & Silver, P. A. Kinetics of spindle pole body separation in budding yeast. Proceedings of the National Academy of Sciences of the United States of America 92, 9707-9711 (1995).

62. Sawin, K. E. & Nurse, P. Identification of fission yeast nuclear markers using random polypeptide fusions with green fluorescent protein. Proceedings of the National Academy of Sciences of the United States of America 93, 15146-15151 (1996).

63. Tarnok, A., Valet, G. K. & Emmrich, F. Systems biology and clinical cytomics: The 10th Leipziger Workshop and the 3rd International Workshop on Slide-Based Cytometry, Leipzig, Germany, April 2005. Cytometry A 69, 36-40 (2006).

64. Fertig, N., Blick, R. H. & Behrends, J. C. Whole cell patch clamp recording performed on a planar glass chip. Biophysical journal 82, 3056-3062 (2002).

65. Schmidt, Mayer, M. & Vogel, H. A Chip-Based Biosensor for the Functional Analysis of Single Ion Channels We thank E. Ermanntraut, L., Giovangrandi, T. Wohland, A. Brecht, M. Kohler, Bieri, D. Stamou, and R. Hovius for advice. This work was supported by the Swiss National Science Foundation (Priority Program for Biotechnology) and by an interdepartmental grant of the Swiss Federal institute of Technology Lausanne (EPFL, Project Microtechnique 96). Angewandte Chemie (International ed 39, 3137-3140 (2000).

66. Seo, J., Ionescu-Zanetti, C., Diamond, J., Lal, R. & Lee, L. P. Integrated multiple patch-clamp array chip via lateral cell trapping junctions. Applied Physics Letters 84, 1973-1975 (2004).

67. Wheeler, A. R. et al. Microfluidic device for single-cell analysis. Analytical chemistry 75, 3581-3586 (2003).

68. Hong, J. W., Studer, V., Hang, G,. Anderson, W. F. Quake, S. R. A nanoliter-scale nucleic acid processor with parallel architecture. Nature biotechnology 22, 435-439 (2004).

69. Wu, H., Wheeler, A. & Zare Chemical cytometry on a picoliter-scale integrated microfluidic chip. Proceedings of the National Academy of Sciences of the United States of America 101, 12809-12813 (2004).

70. Gao, J., Yin, X.F. & Fang Z. L. Integration of single eell injection, cell lysis, separation and detection of intracellular constituents on a microfluidic chip. Lab on a chip 4, 47-52 (2004).

71. Mischel, P. S., Cloughesy, T. F. & Nelson, S. F. DNA-microarray analysis of brain cancer: molecular classification for therapy. Nat Rev Neurosci 5, 782-792 (2004).

72. O'Neil, R. G., Wu, L. & Mullani, N. Uptake of a fluorescent deoxyglucose analog (2-NBDG) in tumor cells. Mol Imaging Biol 7, 388-392 (2005).

73. Zou. C., Wang, Y. & Shen, Z. 2-NBDG as a fluorescent indicator for direct glucose uptake measurement. Journal of biochemical and biophysical methods 64, 207-215 (2005).

### References—Example C

1. Hehlmann, R., Hochhaus. A. & Baccarani, M. Chronic myeloid leukaemia. Lancet 370, 342-50 (2007).

2. Goldman, J. M. & Druker, B. J. Chronic myeloid leukemia: current treatment options. Blood 98, 2039-42 (2001).

3. Mohamed. A. N., Pemberton, P., Zonder, J. & Schiffer. C. A. The effect of imatinib mesylate on patients with Philadelphia chromosome-positive chronic myeloid leukemia with secondary chromosomal aberrations. Clin cancer Res 9, 1333-7 (2003).

4. Shepherd, F. A. et al. Erlotinib in previously treated non-small-cell lung cancer. N Engl J Med 353, 123-32 (2005).

5. Engelman, J. A., Luo, J. & Cantley, L. C. The evolution of phosphatidylinositol 3-kinases as regulators of growth and metabolism. Nat Rev Genet 7, 606-19 (2006).

6. Sharma, S. V., Bell, D. W., Settleman, J. & Haber, D. A. Epidermal growth factor receptor mutations in lung cancer. Nat Rev Cancer 7, 169-81 (2007).

7. Mischel, P. S. & Cloughesy. T. F. Targeted molecular therapy of GBM. Brain Pathol 13, 52-61 (2003).

8. Murren, J. R. Modulating multidrug resistance: can we target this therapy? Clin Cancer Res 8, 633-5 (2002).

9. Levitzki. A. Protein kinase inhibitors as a therapeutic modality. Ace Chem Res 36, 462-9 (2003).

10. Rocha-Lima. C. M., Soares, H. P., Raez, L. E. & Singal, R. EGFR targeting of solid tumors. Cancer Control 14, 295-304 (2007).

11. Cairns, P. et al. Frequent inactivation of PTEN/MMAC1 in primary prostate cancer. Cancer Res 57, 4997-5000 (1997).

12. Pesche, S. et al. PTEN/MMAC1/TEP1 involvement in primary prostate cancers. Oncogene 16, 2879-83 (1998).

13. Feilotter, H. E., Nagai, M. A., Boag, A. H., Eng. C. & Mulligan, L. M. Analysis of PTEN and the 10q23 region in primary prostate carcinomas. Oncogene 16, 1743-8 (1998).

14. Bargonetti, J. & Manfredi, J. J. Multiple roles of the tumor suppressor p53. Curr Opin Oncol 14, 86-91 (2002).

15. McNeil. C. M. et al. c-Myc overexpression and endocrine resistance in breast cancer. J Steroid Biochem Mol Biol 102, 147-55 (2006).

16. Ferretti, G., Felici. A., Papaldo, P., Fabi. A. & Cognetti. F. HER2/neu role in breast cancer: from a prognostic foe to a predictive friend, Curr Opin Obstet Gynecol 19, 56-62 (2007).

17. Shi, Y., Yan, H., Frost, P., Gera, J. & Lichtenstein, A. Mammalian target of rapamycin inhibitors activate the AKT kinase in multiple mycloma cells by up-regulating the insulin-like growth factor receptor/insulin receptor substrate-1/phosphatidylinositol 3-kinase cascade. Mol Cancer Ther 4, 1533-40 (2005).

18. Barabasi, A. L., & Oltvai, Z. N. Network biology; understanding the cell's functional organization. Nat Rev Genet 5, 101-1.3 (2004).

19. Li, J. et al. PTEN, a putative protein tyrosine phosphatase gene mutated in human brain, breast, and prostate cancer. Science 275, 1943-7 (1997).

20. Steck, P. A. et al. Identification of a candidate tumour suppressor gene, MMAC1, at chromosome 10q23.3 that is mutated in multiple advanced cancers. Nat Genet 15, 3.56-62 (1997).

21. Lesehe, R., et al. Cre/loxP-mediated inactivation of the murine Pten tumor suppressor gene. Genesis 32, 148-9 (2002).

22. Freeman D. J. et al. PTEN tumor suppressor regulates p53 protein levels and activity through phosphatase-dependent and -independent mechanisms. Cancer Cell 3, 117-30 (2003).

23. Wu, X. at al. Generation of a prostate epithelial cell-specific Cre transgenic mouse model for tissue-specific gene ablation. Mech Dev 101, 61-9 (2001).

24. Varmus, H. The new era in cancer research. Science 312, 1162-5 (2006).

25. Mittler, M. A., Walters B. C. & Stopa E.G., Observer reliability in histological grading of astrocytoma stereotactic biopsies. J Neurosurg 85, 1091-4 (1996).

26. Chang, J. H. et al. The impact of a multidisciplinary breast cancer center on recommendations for patient management: the University of Pennsylvania experience. Cancer 91, 1231-7 (2001).

27. McGowan, L. & Norris H. J. The mistaken diagnosis of carcinoma of the ovary. Surg Gynecol Obstet 173, 211-5 (1991).

28. Epstein, J. I., Walsh, P. C. & Sanfilippo. F. Clinical and cost impact of second-opinion pathology, Review of prostate biopsies prior to radical prostatectomy. Am J Surg Pathol 20, 851-7 (1996).

29. Arbiser, Z. K., Folpe. A. L. & Weiss, S. W. Consultative (expert) second opinions in soft tissue pathology. Analysis of problem-prone diagnostic situations. Am J Clin Pathol 116, 473-6 (2001).

30 Coblentz T. R., Mills, S. E. & Theodorescu, D. Impact of second opinion pathology in the definitive management of patients with bladder carcinoma. Cancer 91, 1284-90 (2001).

31. Stingl J. & Caldas, C. Molecular heterogeneity of breast cacinomas and the cancer stem cell hypothesis. Nat Rev Cancer 7, 791-9 (2007).

32. Jou, Y. S. et al. Clustering of minimal deleted regions reveals distinct genetic pathways of human hepatocellular carcinoma. Cancer Res 64, 3030-6 (2004).

33. Cells, J. E. et al. Proteomic strategies to reveal tumor heterogeneity among urothelial papillomas, Mol Cell Proteomics 1, 269-79 (2002).

34. Haskill, S., Kivinen, S., Nelson, K. & Fowler, W. C., Jr. Detection of intratumor heterogeneity by simultaneous multiparameter flow cytometric analysis with enzyme and DNA markers. Cancer Res 43, 1003-9 (1983).

35. Schilsky, R. L. Clinical implications of tumor heterogeneity. Haematol Blood Transfus 31, 278-82 (1987).

36. Baselga, J. Targeting tyrosine kinases in cancer: the second wave. Science 312, 1175-8 (2006).

37. Wilhelm, S. at al. Discovery and development of sorafenib: a multikinase inhibitor for treating cancer. Nat Rev Drug Discov 5, 835-44 (2004).

38. Krause, D. S. Van Etten, R. A. Tyrosine kinases as targets for cancer therapy. N Engl J Med 353, 172-87 (2005).

39. Hynes, N. E. & Lane, H. A. ERBB receptors and cancer: the complexity of targeted inhibitors. Nat Rev Cancer 5, 341-54 (2005).

40. Bjornsti, M. A. & Houghton, P. J. The TOR pathway: a target for cancer therapy. Nat Rev Cancer 4, 335-48 (2004).

41. Sebolt-Leopold, J. S. & English, J. M. Mechanisms of drug inhibition of signalling molecules. Nature 441, 457-62 (2006).

42. Sawyers. C. Targeted cancer therapy. Nature 432, 294-7 (2004).

43. Heath, J. R., Phelps, M. E. & Hood, L. NanoSystems biology. Mol Imaging Biol 5, 312-25 (2003).

44. Flood, L., Heath, J. R., Phelps, M.E. & Lin, B. Systems biology and new technologies enable predictive and preventative medicine. Science 306, 640-3 (2004).

45. Pepperkok, R. & Ellenberg, J. High-throughput fluorescence microscopy for systems biology. Nat Rev Mol Cell Biol 7, 690-6 (2006).

46. Megason, S. G. & Fraser, S.E. imaging in systems biology. Cell 130, 784-95 (2007).

47. Toker. A. & Cantley, L. C. Signalling through the lipid products of phosphoinositide-3-OH kinase. Nature 387, 673-6 (1997).

48 Vivanco, I. & Sawyers, C. L. The phosphatidylinositol 3-Kinase AKT pathway in human cancer. Nat Rev Cancer 2, 489-501 (2002).

49. Myers. M. P. et al. The lipid phosphatase activity of PTEN is critical for its tumor supressor function. Proc Natl Acad Sci USA 95, 13513-8 (1998).

50. Li, J. et al. The PTEN/MMAC1 tumor suppressor induces cell death that is rescued by the AKT/protein kinase B oncogene. Cancer Res 58, 5667-72 (1998).

51. Dahia, P. L. et al. PTEN is inversely correlated with the cell survival factor Akt/PKB and is inactivated via multiple mechanismsin haematological malignancies. Hum Mol Genet 8, 185-93 (1999).

52. Sordella, R., Bell, D. W., Haber, D. A. & Settleman, J, Gefitinib-sensitizing EGFR mutations in lung cancer activate anti-apoptotic pathways. Science 305, 1163-7 (2004).

53. Luo, J., Manning, B. D. & Cantley, L. C. Targeting the PI3K-Akt pathway in human cancer: rationale and promise. Cancer Cell 4, 257-62 (2003).

54. Choe, G. et al. Analysis of the phosphatidylinositol 3′-kinase signaling pathway in glioblastoma patients in vivo. Cancer Res 63, 2742-6 (2003).

55. Li, B. et al. Mutant epidermal growth factor receptor displays increased signaling through the phosphatidylinositol-3 kinase/AKT pathway and promotes radioresistance in cells of astrocytic origin. Oncogene 23, 4594-602 (2004).

56. Huang, H. S. et al. The enhanced tumorigenic activity of a mutant epidermal growth factor receptor common in human cancers is mediated by threshold levels of constitutive tyrosine phosphorylation and unattenuated signaling. J Biol Chem 272, 2927-35 (1997).

57. Sun, H. et al. PTEN modulates cell cycle progression and cell survival by regulating phosphatidylinositol 3,4,5,-trisphosphate and Akt/protein kinase B signaling pathway. Proc Natl Acad Sci USA 96, 6199-204 (1999).

58. Stambolic, V. et al. Negative regulation of PKB/Akt-dependent cell survival by the tumor suppressor PTEN. Cell 95, 29-39 (1998).

59. Wu, X., Senechal, K., Neshat, M. S., Whang, Y. E. & Sawyers, C. L. The PTEN/MMAC1 tumor suppressor phosphatase functions as a negative regulator of the phosphoinositide 3-kinase/Akt pathway. Proc Natl Acad Sci USA 95, 15587-91 (1998).

60. Di Cristofario. A. & Pandolfi, P. P. The multiple roles of PTEN in tumor suppression. Cell 100, 387-90 (2000).

61. Hanahan, D. & Weinberg, R. A. The hallmarks of cancer. Cell 100, 57-70 (2000).

62. Feldkamp, M. M., Lala, P., Lau, N. Roncari, L. & Guha, A. Expression of activated epidermal growth factor receptors, Ras-guanosine triphosphate, and mitogen-activated protein kinase in human glioblastoma multiforme specimens. Neurosurgery 45, 1442-53 (1999).

63. Lokker, N. A., Sullivan. C. M., Hollenbach, S. J., Israel, M. A. & Giese, N. A. Platelet-derived growth factor (PDGF) autocrine signaling regulates survival and mitogenic pathways in glioblastoma cells: evidence that the novel PDGF-C and PDGF-D ligands may play a role in the development of brain tumors. Cancer Res 62, 3729-35 (2002).

64. Vogt, P. K., Bader. A. G. & Kang, S. PI 3-kinases: hidden potentials revealed. Cell Cycle 5, 946-9 (2006).

65. Mellinghoff, I. K., et al. Molecular determinants of the response of glioblastomas to EGFR kinase inhibitors. N Engl J Med 353, 2012-24 (2005).

66. Workman, P., Clarke, P. A., Guillard, S. & Raynaud, F. I. Drugging the PI3 kinome. Nat Biotechnol 24, 794-6 (2006).

67. Batra, S. K. et al. Epidermal growth factor ligand-independent, unregulated, cell-transforming potential of a naturally occurring human mutant EGFRvIII gene. Cell Growth Differ 6, 1251-9 (1995).

68. Bell, D. W. et al. Epidermal growth factor receptor mutations and gene amplification in non-small-cell lung cancer: molecular analysis of the IDEAL/INTACT gefitinib trials. J Clin Oncol 23, 8081-92 (2005).

69. Hirsch. F. R. et al. Increased epidermal growth factor receptor gene copy number detected by fluorescence in situ hybridization associates with increased sensitivity to gefitinib in patients with bronchioloalveolar carcinoma subtypes: a Southwest Oncology Group Study, J Clin Oncol 23, 6838-45 (2005).

70. Zhao, J. J. & Roberts. T. M. PI3 kinases in cancer: from oncogene artifact to leading cancer target. Sci STKE 2006, pe52 (2006).

71. Ji, H. et al. Epidermal growth factor receptor variant III mutations in lung tumorigenesis and sensitivity to tyrosine kinase inhibitors. Proc Natl Acad Sci USA 103, 7817-22 (2006).

72. Gomez-Sjoberg, R., Leyrat. A. A., Phone, D. M., Chen. C. S. & Quake, S. R. Versatile, fully automated, microfluidic cell culture system. Anal Chem 79, 8557-63 (2007).

73. Yamazaki, H. et al. Amplification of the structurally and functionally altered epidermal growth factor receptor gene (c-erbB) in human brain tumors. Mol Cell Biol. 8, 1816-20 (1988).

74. Wong. A. J. et al. Structural alterations of the epidermal growth factor receptor gene in human gliomas. Proc Natl Acad Sci USA 89, 2965-9 (1992).

75. Ekstrand. A. J., Sugawa, N., James. C. D. & Collins, V. P. Amplified and rearranged epidermal growth factor receptor genes in human glioblastomas reveal deletions of sequences encoding portions of the N- and/or C-terminal tails. Proc Natl Acad Sci USA 89, 4309-13 (1992).

76. Perez, O. D. & Nolan, G. P. Simultaneous measurement of multiple active kinase states using polychromatic flow cytometry. Nat Biotechnol 20, 155-62 (2002).

77. Krutzik, P. O. & Nolan, G. P. intracellular phospho-protein staining techniques for flow cytometry: monitoring single cell signaling events. Cytometry A. 55, 61-70 (2003).

78. Krutzik, P. O., Crane J. M., Clutter, M. R. & Nolan High-content single-cell drug screening with phosphospecific flow cytometry. Nat Chem Biol. 4, 32-42 (2008).

79. Hale, M. B. & Nolan, G. P. Phospho-specific flow cytometry: intersection of immunology and biochemistry at the Curr Opin Mol Ther 8, 215-24 (2006).

80. Irish, J. M. et al. Single cell profiling of potentiated phospho-protein networks in cancer cells. Cell 118, 217-28 (2004).

81. Sachs, K., Perez, O., Pe'er, D., Lauffenburger, D. A., & Nolan, G. P. Causal protein-signaling networks derived from multiparameter single-cell data. Science 308, 523-9 (2005).

82. Thorsen. T., Maerkl, S. J. & Quake, S. R. Microfluidic large-scale integration. Science 298, 580-4 (2002).

83. Brittain, S., Paul, K., Zhao, X. M. & Whitesides, G. Soft lithography and microfabrication. Physics World 11, 31-36 (1998).

84. Kirby, B. J., Shepodd. T. J. & Hasselbrink, E. F. Voltage-addressable on/off microvalves for high-pressure microchip separations. Journal of Chromatography A 979, 147-154 (2002).

85. Unger, M. A., Chou, H. P., Thorsen. T., Scherer. A. & Quake, S. R. Monolithic microfabricated valves and pumps by multilayer soft lithography. Science 288, 113-6 (2000).

86. Lee. C. C. et al. Multistep synthesis of a radiolabeled imaging probe using integrated microfluidics. Science 310, 1793-6 (2005).

87. Wang, J. et al. Integrated microfluidics for parallel screening of an in situ click chemistry library. Angew Chem Int Ed Engl 45, 5276-81 (2006).

88. Yu, Z. T. K. et al. An integrated microfluidic chip for parallel culture and multiparametric analysis of murine and human cell lines. (in revision).

89. Kamei, K. et al. An integrate microfluidic culture device for quantitative analysis of human embryonic stem cells. (submitted).

90. Chin, V. I. et al. Microfabricated platform for studying stern cell fates, Biotechnol Bioeng 88, 399-415 (2004).

91. Takayama, S. et al. Patterning cells and their environments using multiple laminar fluid flows in capillary networks. Proc Nall Acad Sci USA 96, 5545-8 (1999).

92. El-Ali, J., Sorger, P. K. & Jensen, K. F. Cells on chips. Nature 442, 403-11 (2006).

93. Gu. W., Zhu, X., Futai, N., Cho, B. S. & Takayama, S. Computerized microfluidic cell culture using elastomeric channels and Braille displays. Proc Natl Acad Sci USA 101, 15861-6 (2004).

94. Chung, B. G. et al. Human neural stem cell growth and differentiation in a gradient-generating microfluidic device. Lab Chip 5, 401-6 (2005).

95. Kim, L., Vahey, M. D., Lee. FLY. & Voldman, J. Microfluidic arrays for logarithmically perfused embryonic stem cell culture. Lab Chip 6, 394-406 (2006).

96. Abhyankar, V. V. & Beebe, D. J. Spatiotemporal micropatterning of cells on arbitrary substrates. Anal Chem 79, 4066-4073 (2007).

97. Abhyankar, V. V., Bittner, Causey, J. A. & Kamp. T. J. B., D. J. HUMAN EMBRYONIC STEM CELL CULTURE IN MICROFLUIDIC CHANNELS, 7th International Conference on Miniaturized Chemical and Biochemical Analysts Systems, 17 (2003).

98. Nevill, J. T., Cooper, R., Dueck, M., Breslauer, D. N. & Lee, L. P. Integrated microfluidic cell culture and lysis on a chip. Lab Chip 7, 1689-95 (2007).

99. Zhang, M. Y. et al. Microfluidic environment for high density hepatocyte culture. Biomed Microdev 10, 117-21 (2008).

100. Lee, P. J., Hung, P. J., Rao, V. M. & Lee, L. P. Nanoliter scale microbioreactor array for quantitative cell biology. Biotechnol Bioeng 94, 5-14 (2006).

101. Di Carlo, D., Wu, L. Y. & Lee, L. P. Dynamic single cell culture array: Lab Chip 6, 1445-9 (2006).

102. Hung, P. J., Lee, P. J., Sabounchi, P., Lin, R. & Lee, L. P. Continuous perfusion microfluidic cell culture array for high-throughput cell-based assays. Biotechnol Bioeng 89, 1-8 (2005).

103. Yu, H., Meyvantsson, L., Shkel, I. A. & Beebe, D. J. Diffusion dependent cell behavior in microenvironments. Lab Chip 5, 1089-95 (2005).

104. Yu, H., Alexander. C. M. & Beebe, D. J. A plate reader-compatible microchannel array for cell biology assays. Lab Chip 7, 388-91 (2007).

105. Starkuviene, V. & Pepperkok, R. The potential of high-content high-throughput microscopy in drug discovery. Br J Pharmacol 152, 62-71 (2007).

106. Carpenter. A. E. Image-based chemical screening. Nat Chem Biol 3, 461-5 (2007).

107. Lei, Q. et al. NKX3.1 stabilizes p53, inhibits AKT activation, and blocks prostate cancer initiation caused by PTEN loss. Cancer Cell 9, 367-78 (2006).

108. Wang, S. et al. Prostate-specific deletion of the murine Pten tumor suppressor gene leads to metastatic prostate cancer. Cancer Cell 4, 209-21 (2003).

109. Wang, S. et al. Pten deletion leads to the expansion of a prostatic stem/progenitor cell subpopulation and tumor initiation. Proc Natl Acad Sci USA 103, 1480-5 (2006).

110. Wang, M. Y. et al. Mammalian target of rapamycin inhibition promotes response to epidermal growth factor receptor kinase inhibitors in PTEN-deficient and PTEN-intact glioblastoma cells. Cancer Res 66, 7864-9 (2006).

111. Liliental, J. et al. Genetic deletion of the Pten tumor suppressor gene promotes cell motility by activation of Rac1 and Cdc42 GTPases. Curr Biol 10, 401-4 (2000).

112. Stiles, B. et al. Essential role of AKT-1/protein kinase B alpha in PTEN-controlled tumorigenesis. Mol Cell Biol 22, 3842-51 (2002).

113. Li, G. et al. PTEN deletion leads to up-regulation of a secreted growth factor pleiotrophin. J Biol Chem 281, 10663-8 (2006).

114. Shen. W. H. et al. Essential role for nuclear PTEN in maintaining chromosomal integrity. Cell 128, 157-70 (2007).

115. Jiao, J. et al. Murine cell lines derived from Pten null prostate cancer show the critical role of PTEN in hormone refractory prostate cancer development. Cancer Res 67, 6083-91 (2007).

116. Chang. C. J., Freeman, D. J. & Wu, H. PTEN regulates Mdm2 expression through the P1 promoter. J Biol Chem 279, 29841-8 (2004).

117. Yu, Y. & Bradley. A. Engineering chromosomal rearrangements in mice. Nat Rev Genet 2, 780-90 (2001).

118. Gurney, J. G. & Kadan-Lottick, N. Brain and other central nervous system tumors: rates, trends, and epidemiology. Curr Opin Oncol 13, 160-6 (2001).

119. Smith, J. S. et al. PTEN mutation, EGFR amplification, and outcome in patients with anaplastic astrocytoma and glioblastoina multiforme. J Natl Cancer Inst 93, 1246-56 (2001).

120. Ermoian, R. P. et al. Dysregulation of PTEN and protein kinase B is associated with glioma histology and patient survival. Clin Cancer Res 8, 1100-6 (2002).

### FURTHER REFERENCES

1. Laks, D. R.*, Masterman-Smith, M*, Visnyei, K., Angenieux, B., Liau, L., Lazareff, J., Mischel, P. S., Cloughesy, T., Horvath, S., Kornblum, H. I. Neuroshere formation is an independent predictor of clinical outcome in malignant glioma. Stem Cells (In Press) * Both authors contributed equally to this work.

2. Nakano, Masterman-Smith, M., Saigusa, K., Paucar, A., Horvath, S., Shoemaker, L., Watanabe, M., Negro, A., Bajpal, R. Howes, A., Lelievre, V., Waschek, J. A., Lazareff, J. A., Freije, W. A., Liau, L. M., Gilbertson, R. J., Cloughesy, T. F., Geschwind, D. H., Nelson, S. F., Mischel, P. S., terskikh, A. V., Kornblum, H. I. Maternal embryonic leucine zipper kinase is a key regulator of the proliferation of malignant brain tumors, including brain tumor stem cells. Journal of Neuroscience Research 2008 86:48-60.

3. Komblum, H. I., Geschwind, D. H., Nakano, I., Dougherty, J. D., Lazareff, J. A., Mischel, P. S., Masterman-Smith, M., Huang, J. “Compositions and Methods for Diagnosing and Treating Brain Cancer and Identifying Neural Stem Cells.” U. S. application Ser. No. 11/576,444 based on international patent application PCT/US05/035355

### Publications in 2007

1. Parsa A, Waldron J, Panner A, Crane C, Parney I, Barry J, Cachola K, Murray J, Jensen M, Mischel P S, Stokoe D and Pieper R. (2007) Loss of tumor suppressor PTEN function increases B7-H1 expression and immunoresistance in glioma. Nature Medicine, 13(1):84-8.

2. Mellinghoff I K, Cloughesy T F, Mischel P S (2007). PTEN loss as a mechanism of resistance to EGFR tyrosine kinases inhibitors. Clinical Cancer Research, 13(2):378-381.

3. Thomas R K, Baker A C, DeBiasi R M, Winckler W, LaFramboise T, Lin W M, Wang M, Feng W, Zander T, MacConnaill L E, Lee J C, Nicoletti R, Hatton C, Goyette M, Girard L, Majmudar K, Ziaugra L, Wong K, Gabriel K, Beroukhim R, Peyton M, Barretina J, Dutt, A, Emery C, Greulich H, Shah K, Sasaki H, Gazdar A, Minna J, Armstrong SA, Mellinghoff IK, Hodi FS, Dranoff G, Mischel P S, Cloughesy T F, Nelson S F, Liau L M, Mertz K, Rubin M S, Moch H, Loda M, Catalona W, Fletcher J, Signoretti S, Kaye F, Anderson K C, Demetri G D, Dummer R, Wagner S, Herlyn M, Sellers W R, Meyerson M, and Garraway L A (2007). High-throughput oncogene mutation profiling in human cancer. Nat Genet 39(3):347-51.

4. Carlson M R, Pope W B, Horvath S, Braunstein J G, Nghiemphu P, Tso C L, Mellinghoff I, Lai A, Liau L M, Mischel P S, Dong J, Nelson S F, Cloughesy T F. (2007) Relationship between survival and edema in malignant gliomas: role of vascular endothelial growth factor and neuronal pentraxin 2. Clin Cancer Res. 13(9):2592-8.

5. Nakano I, Masterman-Smith M, Saigusa K, Paucar A A, Horvath S, Shoemaker L, Watanabe M, Negro A, Bajpai R, Howes A, Lelievre V, Waschek J A, Lazareff J A, Freije W A, Liau L M, Gilbertson R J, Cloughesy T F, Geschwind D H, Nelson S F, Mischel P S, Terskikh A V, Kornblum HI. (2007) Maternal embryonic leucine zipper kinase is a key regulator of the proliferation of malignant brain tumors, including brain tumor stem cells J Neurosci Res.

6. Beroukhim R, Getz G, Nghiemphu L, Barretina J, Hsueh T, Linhart D, Vivanco I, Lee J C, Huang J H, Alexander S Du J. Tweeny K, Thomas R K, Shah K, Soto H, Perrier S, Prensner J, Debiasi R M, Demichelis F, Hatton C, Rubin M A, Garraway L A, Nelson S F, Liau L M, Mischel P S, Cloughesy T F, Meyerson M, Golub T A, Lander E S, Mellinghoff I K, Sellers W R. (2007) Assessing the Significance of Chromosomal Aberrations in Cancer: Methodology and Application to Glioma, PNAS, in press.

7. Yoshimoto K, Dang J, Zhu S, Nathanson D, Huang T, Dumont R, Seligson D B, Yong W H, Xiong Z, Rao N, Winter H, Chakravarti A, Bigner D D, Mellinghoff I K, Horvath S, Cavence W K, Cloughesy T F, Mischel P S. (2007) Development of the real-time RT-PCR assay for detecting EGFRvIII in formalin-fixed paraffin-embedded glioblastoma samples. Clinical Cancer Research, in press.

8. Cloughesy T F, Koji Yoshimoto K, Nghiemphu P, Brown K, Dang J, Zhu S, Huseh T, Chen Y, Wang W, Youngkin D, Liau L, Martin N, Becker D, Bergsneider M, Lai A, Green R, Oglesby T, Koleto M, Trent J, Horvath S. Mischel P S*, Mellinghoff IK* Sawyers CL* (*Co-Senior and corresponding authors). (2007) Antitumor activity of rapamycin in patients with recurrent PTEN-deficient glioblastoma PLoS Medicine, in press

9. Under review: Lu, K V, Zhu S J, Dang J, Yoshimoto K, Felciano R M, Richards D R, Laurance M E, Chen Z, Caldwell J S, Shah N P, Horvath S, Nelson S F, James C D, Bergers G, Lee F, Weinmann R, Mischel P S. The Src Tyrosine Kinases Fyn and Lyn are Dasatinib-Sensitive Molecular Target in Glioblastoma Patients Submitted; revised manuscript under consideration at Cancer Cell.

1. A microfluidic system, comprising:
a pipette system comprising a plurality of pipettes;
a microfluidic chip arranged proximate said pipette system;
an imaging optical detection system arranged proximate said microfluidic chip; and
an image processing system in communication with said imaging optical detection system,
wherein said microfluidic chip comprises a plurality of cell culture chambers defined by a body of said microfluidic chip, each cell culture chamber being in fluid connection with an input channel and an output channel defined by said microfluidic chip, and
wherein said pipette system is constructed and arranged to at least one of inject fluid through said plurality of pipettes into said plurality of input channels or extract fluid through said plurality of pipettes from said plurality of output channels while said microfluidic system is in operation.

2. A microfluidic system according to claim 1, wherein each cell culture chamber of said plurality of cell culture chambers has a volume of at least about 200 pL and less than about 2.4 μL.

3. A microfluidic system according to claim 1, wherein said microfluidic chip comprises at least about 10 cell culture chambers.

4. A microfluidic system according to claim 1, wherein said imaging system has a resolution sufficient to resolve images of separate biological cells of interest to be cultured in said cell culture chambers.

5. A microfluidic system according to claim 1, wherein said body of said microfluidic chip comprises a PDMS layer attached to a substrate.

6. A microfluidic system according to claim 5, wherein said substrate comprises a layer of an extra-cellular matrix component coated thereon to provide immobilization of biological cells of interest.

7. A microfluidic system according to claim 1, further comprising an illumination system constructed and arranged to have an optical path to said microfluidic chip, said illumination system being suitable to illuminate cells cultured in said cell culture chambers while in operation.

8. A microfluidic system according to claim 7, wherein said illumination system provides white light illumination of said microfluidic chip.

9. A microfluidic system according to claim 7, wherein said illumination system comprises a laser to illuminate at least portions of said microfluidic chip with substantially monochromatic light at a desired wavelength.

10. A microfluidic system according to claim 9, wherein said imaging optical detection system is constructed to detect at least one of elastically scattered, fluorescent and inelastically scattered light from said laser of said illumination system after illuminating at least a portion of said microfluidic chip.

11. A microfluidic system according to claim 7, wherein said image processing system is adapted to analyze data from said imaging optical detection system that is indicative of at least one of a biological function or an effect of a biologically active material on individual biological cells cultured in said plurality of cell culture chambers.

12. A microfluidic system according to claim 1, wherein said pipette system is an automated, robotic pipette system.

13. A method of automated fluorescent imaging of a plurality of cell cultures, said method comprising:
i) loading a plurality of cell cultures into a plurality of cell culture chambers of a microfluidic chip, said plurality of cell culture chambers comprising a surface coating of an extracellular matrix material for immobilization of said cell cultures;
ii) applying at least one fluorescent probe to said cell cultures, and incubating the cell cultures under suitable conditions to promote binding of said probe to a specific target in or on the cells;
iii) illuminating said cell cultures to cause said fluorescent probe to emit fluorescent light; and
iv) imaging light fluorescing from said cells in each of said plurality of cell culture chambers while said cell cultures remain substantially immobilized in said plurality of cell culture chambers to provide information regarding said specific target in or on said cells.

14. The method of claim 13 wherein unbound extracellular fluorescent probe is removed by washing prior to step iv.

15. The method of claim 13 wherein a plurality of fluorescent probes is applied to the cell cultures.

16. The method of claim 13 wherein the extracellular matrix material is selected from the group consisting of fibronectin, laminin, Matrigel and RGD peptide.

17. The method of claim 13, additionally comprising the step of
v) processing the images obtained in step iv to obtain flow-cytometry-like data.

18. The method of claim 17, additionally comprising the step of
vi) processing the flow-cytometry-like data to obtain a heat map of pathway differentiation.

19. The method of claim 17 wherein the flow-cytometry-like data is a 2-D or 3-D dot plot or a histogram.

20. The method of claim 13, wherein at least one cell culture comprises cancer cells.

21. The method of claim 20, wherein the cancer cells are mammalian glioblastoma cells or breast cancer cells.

22. The method of claim 21 wherein the mammalian glioblastoma cells are selected from the group consisting of glioblastoma cell line U87 and genetically modified U87 cells (U87-PTEN, U87-EGFR, U87 EGFRvIII and U87-EGFRvIII/PTEN).

23. The method of claim 21 wherein the breast cancer cells are selected from the group consisting of MDA453 (ErbB2+), MDA361 (ErbB2+), BT474 (ErbB2++), SKBr3 (ErbB2++) and JIMT1 (ErbB2++, PTEN−).

24. The method of claim 13 wherein the volume of each chamber is between 2 pL and 2.4 μL.

25. The method of claim 24 wherein the volume is about 150 nL.

26. A method of determining at least one target characteristic of a cell type, said method comprising
i) loading a substantially pure culture of said cell type into a cell culture chamber of the system of claim 1; and
ii) assaying the culture under suitable conditions to determine at least one target characteristic of said cell type.

27. The method of claim 26 wherein a plurality of cell cultures is assayed.

28. The method of claim 26 wherein said target characteristic is selected from the group consisting of EGFRvIII, PTEN, mTOR, glucose uptake, FDG uptake.

29. The method of claim 26, wherein a plurality of characteristics are determined.
